(19) United States
(12) Patent Application Publication
(10) Pub. No.: US 2026/0027181 A1
(43) Pub. Date: Jan. 29, 2026

(54) Administration of Engineered T Cells for Treatment of Cancers in the Central Nervous System

(71) Applicant: City of Hope, Duarte, CA (US)

(72) Inventors: Behnam Badie, Duarte, CA (US); Christine E. Brown, Duarte, CA (US); Stephen J. Forman, Duarte, CA (US); Saul J. Priceman, Duarte, CA (US)

(21) Appl. No.: 19/351,506

(22) Filed: Oct. 7, 2025

Related U.S. Application Data
(63) Continuation of application No. 17/025,723, filed on Sep. 18, 2020, now Pat. No. 12,472,233.
Application No. 17/025,723 is a continuation of application No. 15/425,773, filed on Feb. 6, 2017, now abandoned.
(60) Provisional application No. 62/292,152, filed on Feb. 5, 2016.
(60) Provisional application No. 62/309,348, filed on Mar. 16, 2016.

(51) Int. Cl. A61K 38/17; A61K 9/00; A61K 38/20; A61K 40/11; A61K 40/31; A61K 40/42; A61P 35/00; C07K 14/54; C07K 14/705; C07K 14/725; C07K 14/73; C12N 5/0783

(52) U.S. Cl. CPC A61K 38/1774; A61K 9/0085; A61K 38/177; A61K 38/2086; A61K 40/11; A61K 40/31; A61K 40/4205; A61K 40/4217; A61P 35/00; C07K 14/5437; C07K 14/7051; C07K 14/70514; C07K 14/70521; C12N 5/0636; A61K 2239/17; A61K 2239/31; A61K 2239/38; A61K 2239/47; A61K 2239/49; C07K 2319/03; C07K 2319/33; C07K 2319/74

(57) ABSTRACT

An improved method of treating cancers with engineered T cells is described.

### CROSS-REFERENCE TO RELATED APPLICATIONS

This application is a continuation of U.S. application Ser. No. 17/025,723, filed Sep. 18, 2020, which is a continuation of U.S. application Ser. No. 15/425,773, filed Feb. 6, 2017, which claims the benefit of prior U.S. Provisional Application Ser. No. 62/292,152, filed Feb. 5, 2016, and of prior U.S. Provisional Application Ser. No. 62/309,348, filed Mar. 16, 2016. The disclosures of the above applications are hereby incorporated by reference in their entirety.

### SEQUENCE LISTING

This application contains a Sequence Listing that has been submitted electronically as an XML file named “40056-0028003_SL_ST26.XML.” The XML file, created on Oct. 6, 2025, is 87,234 bytes in size. The material in the XML file is hereby incorporated by reference in its entirety.

### BACKGROUND

Tumor-specific T cell based immunotherapies, including therapies employing engineered T cells and ex vivo expanded or selected T cells, have been investigated for anti-tumor treatment. In some cases, the T cells used in such therapies do not remain active in vivo for a long enough period. In some cases, the tumor-specificity of the T cells is relatively low. In some cases, the engineered T cells have insufficient access to the tumor. Therefore, there is a need in the art for tumor-specific cancer therapies with more effective anti-tumor function.

Treatment of cancers of the central nervous system can be particularly challenging. For example, treatment of high-grade malignant glioma (MG), including anaplastic astrocytoma (AA-grade III) and glioblastoma multiforme (GBM-grade IV), remains a significant therapeutic challenge. Currently available therapeutic options have limited curative potential and only less than 5% of patients survive more than five years after initial diagnosis.

### SUMMARY

Described herein are methods for treating malignancies in the central nervous system by administering compositions comprising T cells (e.g., CAR T cells, Tumor Infiltrating lymphocytes (“TIL”), TCR-engineered T cells, or T cell clones) to the cerebrospinal fluid (“CSF”) of a patient. The T cells include T cells that have be manipulated, for example, by introduction of a nucleic acid molecule expressing a desired receptor, by ex vivo expansion of isolated or genetically-modified T cells or by ex vivo selection of a subset of T cells obtained from a patient or a donor or by a combination of two or more of these techniques. Administration to the CNS can be accomplished, for example, by administration to the ventricular system or the central cavity of the spinal column. Administration to the CNS, as the term is used herein, is distinct from both intratumoral administration (injection or infusion into the tumor itself) and administration to a cavity created by resection of a tumor. However, the CNS administration methods described herein can be combined with intraturmoral and/or post-resection, intracavity administration.

The CNS administration described herein permits infusion of relatively large volumes of the composition comprising T cells, for example 1 ml-2 ml or more in a single infusion. Thus, several million T cells can be administered in a single infusion.

A method of treating a patient diagnosed with a malignancy of the central nervous system is thus disclosed, which comprises infusing a composition comprising an effective amount of T cells into an anatomical compartment of a patient diagnosed with a malignancy of the central nervous system, the anatomical compartment containing cerebrospinal fluid (“CSF”). The method includes infusion of a composition into a ventricular system or a portion of a central canal of a spinal cord, for example. In one embodiment of the disclosed method, the malignancy of the central nervous system includes a primary tumor or a metastasized tumor found somewhere in the central nervous system, including a portion of the brain, spinal column, or the like. Preferably the anatomical compartment contains a contiguous volume of at least about 50, 100, or 150 mL of cerebrospinal fluid.

The manipulated T cells infused in the methods described herein target tumor antigens, for example surface protein and intracellular proteins. The malignancies treated can be primary tumors or secondary tumors arising from cancers originating elsewhere in the body. Because administration to the cerebrospinal fluid allows the T cells access to regions beyond the local site of injection, the methods described herein can be used to attack and reduce the size of tumors remote from the site of injection but within the CNS. TCR-engineered T cells are prepared by introduction of TCRaP genes into T cells (e.g., autologous T cells) followed by ex vivo expansion of T cells; and infusion of T cells into the patient. The infusion of the TCR-engineered T cells confers tumor reactivity to patients whose tumor expresses the appropriate antigen and HLA restriction element. The TCR can be targeted to any of a variety of tumor antigens, including, for example melanoma-associated antigen recognized by T cells 1 (MART-1), glycoprotein (gp) 100, carcinoembryonic antigen (CEA), p53, melanoma-associated antigen (MAGE-A3, and New York esophageal squamous cell carcinoma antigen (NYESO).

Described herein is a method of treating a patient diagnosed with a malignancy of the central nervous system comprising introducing into the cerebrospinal fluid (CSF) of the patient a composition comprising an effective amount of T cells.

In various embodiments: the T cells are autologous or allogenic T cells; the T cells have been manipulated ex vivo by one or more of: expansion, fractionation or transfection with a recombinant nucleic acid molecule; the T cells comprise cells that have been transfected with a recombinant nucleic acid molecule encoding a polypeptide that binds to a tumor cell antigen; the polypeptide is a chimeric antigen receptor; the composition is administered intraventricularly; the composition is administered to the central canal of the spinal cord; the administration is to the left ventrical or the right ventrical; the composition comprises at least 1×10⁶ cells; the composition comprising T cells is administered at least two times; the administrations differ in the total number of T cells administered; the administrations escalate in dose; the administrations de-escalate in dose; the T cells comprise CAR T cells; the T cells comprise autologous tumor infiltrating lymphocytes; the T cells comprise TCR-engineered T cells; the malignancy is a diffuse, infiltrating tumor; the malignancy is a primary brain tumor; one or more tumor foci decrease in size by at least 25%; the malignancy arose from a primary cancer selected from: breast cancer, lung cancer, head and neck cancer, and melanoma; the method is performed after tumor resection; the method further comprises intratumoral administration of a composition comprising T cells; the malignancy is secondary brain tumor; the method further comprises intratumoral administration of a composition comprising therapeutic T cells expressing a chimeric antigen receptor that binds a protein expressed on the surface of glioblastoma cells; the patient has previously undergone resection of a tumor lesion; the tumor antigen is selected from the group consisting of: IL13Rα2, HER2, PSCA, EGFR, EGFRvIII, EphA2, NY-ESO-1, and CD19; T cells comprise both CD4+ cells and CD8+ cells; the T cells have undergone ex vivo expansion; the T cells comprise at least 10% T_{CM} cells; at least 40%, 50%, 60%, 70% or more of the cells infused are CD4+; at least at least 40%, 50%, 60%, 70% or more of the cells infused express a cell surface receptor that targets the tumor antigen (e.g., IL13Rα2); and the dose of cells is based on the number of infused cells that express a cell surface receptor that targets the tumor antigen (e.g., IL13Rα2).

In some embodiments the T cells comprise CART cells that target IL13Rα2 and the cells comprise a nucleic acid molecule encoding a chimeric antigen receptor comprising: human IL-13 or a variant thereof having 1-10 amino acid modifications; a transmembrane domain selected from: a CD4 transmembrane domain or variant thereof having 1-10 amino acid modifications, a CD8 transmembrane domain or variant thereof having 1-10 amino acid modifications, a CD28 transmembrane domain or a variant thereof having 1-10 amino acid modifications, and a CD3ζ transmembrane domain or a variant thereof having 1-10 amino acid modifications; at least one costimulatory domain; and CD3ζ signaling domain of a variant thereof having 1-10 amino acid modifications. In some embodiments: the costimulatory domain is selected from the group consisting of: a CD28 costimulatory domain or a variant thereof having 1-10 amino acid modifications, a 4IBB costimulatory domain or a variant thereof having 1-10 amino acid modifications and an OX40 costimulatory domain or a variant thereof having 1-10 amino acid modifications; the variant of a human IL13 has 1-10 amino acid modification that increase binding specificity for IL13Rα2 versus IL13Rα1; the human IL-13 or variant thereof is an IL-13 variant comprising the amino acid sequence of SEQ ID NO:3 with 1 to 5 amino acid modifications, provided that the amino acid at position 11 of SEQ ID NO:3 is other than E; the chimeric antigen receptor comprises two different costimulatory domains selected from the group consisting of: a CD28 costimulatory domain or a variant thereof having 1-10 amino acid modifications, a 4IBB costimulatory domain or a variant thereof having 1-10 amino acid modifications and an OX40 costimulatory domain or a variant thereof having 1-10 amino acid modifications; the chimeric antigen receptor comprises two different costimulatory domains selected from the group consisting of: a CD28 costimulatory domain or a variant thereof having 1-2 amino acid modifications, a 4IBB costimulatory domain or a variant thereof having 1-2 amino acid modifications and an OX40 costimulatory domain or a variant thereof having 1-2 amino acid modifications; the chimeric antigen receptor comprises: human IL-13 or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ζ transmembrane domain or a variant thereof having 1-2 amino acid modifications; a costimulatory domain; and CD3ζ signaling domain of a variant thereof having 1-2 amino acid modifications; the CAR comprises a spacer region located between the IL-13 or variant thereof and the transmembrane domain; the spacer region comprises an amino acid sequence selected from the group consisting of SEQ ID NO: 4, 14-20, 50 and 52; the chimeric antigen receptor comprises an amino acid sequence selected from the group consisting of SEQ ID NOs: 10 and 31-48.

In some embodiments the T cells express a chimeric antigen receptor that binds HER2 comprise a nucleic acid molecule encoding a chimeric antigen receptor comprising: a HER2 targeting sequence; a transmembrane domain selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications, and a CD3s transmembrane domain or a variant thereof having 1-5 amino acid modifications; a costimulatory domain selected from a CD28 costimulatory domain or a variant thereof having 1-5 amino acid modifications and a 4-IBB costimulatory domain or a variant thereof having 1-5 amino acid modifications; and CD3s signaling domain of a variant thereof having 1-5 amino acid modifications. In certain embodiments: the HER2 targeting domain is a HER2 scFv; the HER2 scFv comprising the amino acid sequence: DIQMTQSPSSLSASVGDRVTITCRASQDVNTAVAWYQQKPGKAPKLLIYSASFLYSGVPSRFS GSRSGTDFTLTISSLQPEDFATYYCQQHYTTPPTFGQGTKVEIKGSTSGGGSGGGSGGGGSSE VQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:49) or a variant thereof having 1 to 5 amino acid modifications; the chimeric antigen receptor comprises: a HER2 targeting sequence; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3s transmembrane domain or a variant thereof having 1-2 amino acid modifications; a costimulatory domain selected from a CD28 costimulatory domain or a variant thereof having 1-2 amino acid modifications and a 4-IBB costimulatory domain or a variant thereof having 1-2 amino acid modifications; and CD3 signaling domain of a variant thereof having 1-2 amino acid modifications; the nucleic acid molecule expresses a polypeptide comprising an amino acid sequence selected from SEQ ID NO: 26 and 27 or a variant thereof having 1-5 amino acid modifications.

Also described herein is a method of treating a patient diagnosed with a malignancy of the central nervous system comprising infusing a composition comprising an effective amount of T cells into an anatomical compartment of a patient diagnosed with a malignancy of the central nervous system, the anatomical compartment containing cerebrospinal fluid (CSF). In various embodiments: the anatomical compartment comprises a portion of a ventricular system; the anatomical compartment comprises a portion of a central canal of a spinal cord; the malignancy of the central nervous system includes a brain tumor; the malignancy of the central nervous system includes a metastasized tumor; the anatomical compartment contains a contiguous volume of at least about 50 mL of cerebrospinal fluid; the anatomical compartment contains a contiguous volume of at least about 100 mL of cerebrospinal fluid; and the anatomical compartment contains a contiguous volume of at least about 150 mL of cerebrospinal fluid.

Among the cancers that can be treated by the methods described herein are primary CNS malignancies and secondary malignancies arising from a cancer located elsewhere, for example. Acute Lymphoblastic Leukemia (ALL), Acute Myeloid Leukemia (AML), Adrenocortical, Carcinoma, AIDS-Related Cancers, Anal Cancer, Appendix Cancer, Astrocytomas, Atypical Teratoid/Rhabdoid Tumor, Central Nervous System, Basal Cell Carcinoma, Bile Duct Cancer, Bladder Cancer, Bone Cancer, Osteosarcoma and Malignant Fibrous Histiocytoma, Brain Stem Glioma, Brain Tumors, Breast Cancer, Bronchial Tumors, Burkitt Lymphoma, Carcinoid Tumors, Central Nervous System Cancers, Cervical Cancer, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Embryonal Tumors, Central Nervous System, Endometrial Cancer, Ependymoblastoma, Ependymoma, Esophageal Cancer, Esthesioneuroblastoma, Ewing Sarcoma Family of Tumors Extracranial Germ Cell Tumor, Extragonadal Germ Cell Tumor Extrahepatic Bile Duct Cancer, Eye Cancer Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST)—see Soft Tissue Sarcoma, Germ Cell Tumor, Gestational Trophoblastic Tumor, Glioma, Hairy Cell Leukemia, Head and Neck Cancer, Heart Cancer, Hepatocellular (Liver) Cancer, Histiocytosis, Hodgkin Lymphoma, Hypopharyngeal Cancer, Intraocular Melanoma, Islet Cell Tumors (Endocrine Pancreas), Kaposi Sarcoma, Kidney cancer, Langerhans Cell Histiocytosis, Laryngeal Cancer, Leukemia, Lip and Oral Cavity Cancer, Liver Cancer (Primary), Lobular Carcinoma In Situ (LCIS), Lung Cancer, Lymphoma, Macroglobulinemia, Male Breast Cancer, Malignant Fibrous Histiocytoma of Bone and Osteosarcoma, Medulloblastoma, Medulloepithelioma, Melanoma, Merkel Cell Carcinoma, Mesothelioma, Metastatic Squamous Neck Cancer with Occult Primary Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma, Multiple, Myeloproliferative Disorders, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma and Malignant Fibrous Histiocytoma of Bone, Ovarian Cancer, Pancreatic Cancer, Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Sézary Syndrome, Small Cell Lung Cancer, Small Intestine Cancer, Soft Tissue Sarcoma, Squamous Cell Carcinoma, Squamous Neck Cancer, Stomach (Gastric) Cancer, Supratentorial Primitive Neuroectodermal Tumors, T-Cell Lymphoma, Cutaneous, Testicular Cancer, Throat Cancer, Thymoma and Thymic Carcinoma, Thyroid Cancer, Transitional Cell Cancer of the Renal Pelvis and Ureter, Trophoblastic Tumor, Ureter and Renal Pelvis Cancer, Urethral Cancer, Uterine Cancer, Uterine Sarcoma, Vaginal Cancer, Vulvar Cancer, Waldenstrom Macroglobulinemia, and Wilms Tumor.

In some embodiments, the malignancy treated according to the disclosed methods comprises a tumor. In some embodiments, treatment results in at least a 50% reduction in tumor volume, at least a 60% reduction in tumor volume, at least a 70% reduction in tumor volume, at least an 80% reduction in tumor volume, or at least an 90% reduction in tumor volume. And in some embodiments, the treatment results in elimination of the malignancy.

In some embodiments, the patient does not experience any grade 3 or higher toxicity.

In some embodiments, the patient was administered a regimen of steroids prior to treatment with the composition comprising an effective amount of T cells, and in some embodiments the regimen of steroids is reduced to a lower dose following the treatment.

In some embodiments, the patient has an increased life expectancy compared to a patient receiving standard of care treatment, including radiation therapy, small molecule drug therapy, antibody therapeutics, or a combination thereof. And in some embodiments, in which the patient receiving standard of care (“SOC”) treatment can expect to survive about 15 months from initial diagnosis (overall survival or OS), the patient receiving the disclosed treatment can expect an OS of 15, 20, 25, 26, 27, 28, 29, 30, 31, 32, 33, 34, 35, 36 months or more. In some embodiments, the patient receiving the claimed treatment can expect an OS of 42, 48, 54, 60, 66, 72, 78, 84, 90 months or more.

In some embodiments, the composition comprises at least 2×10⁶ T cells or 2×10⁶ T cells expressing a cell surface receptor targeting a tumor antigen, while in some embodiments, the composition comprises at least 1×10⁶ T cells or 1×10⁶ T cells expressing a cell surface receptor targeting a tumor antigen. In some embodiments, the composition comprises at least 5×10⁶ T cells or 5×10⁶ T cells expressing a cell surface receptor targeting a tumor antigen, while in some embodiments, the composition comprises at least 10×10⁶ T cells or 10×10⁶ T cells expressing a cell surface receptor targeting a tumor antigen. In some embodiments, the disclosed methods comprised repeated administrations of the compositions, for instance repeating administration of the composition at least five times, repeating administration of the composition at least ten times, or repeating administration until the patient receives a total dose of at least 90×10⁶ T cells or T cells expressing a cell surface receptor targeting a tumor antigen. In some embodiments, the administration is repeated once a week or once every two weeks. In some embodiments, the repeated administrations are continued over the course of 15 weeks.

Also disclosed herein are methods of increasing a level of at least one cytokine or chemokine in the cerebrospinal fluid (CSF) of a patient comprising, administering a composition comprising an effective amount of T cells into the CSF of a patient with a malignancy of the central nervous system, wherein the level of at least one cytokine or chemokine in the CSF is increased following administration of the composition comprising an effective amount of T cells compared to a baseline level of the at least one cytokine or chemokine prior to the administration.

In some embodiments of the disclosed methods, the level of the at least one cytokine or chemokine in the CSF following the administration is increased 10-fold or 5-fold compared to the baseline level.

In some embodiments, the level of at least five or at least ten cytokines or chemokines is increased following administration of the composition comprising an effective amount of T cells compared to a baseline level of the at least five cytokines or chemokines prior to the administration. In some embodiments, the at least one cytokine or chemokine comprises EGF, Eotaxin, FGF, G-CSF, GM-CSF, HGF, IFN-α, IFN-γ, IL-10, IL-12, IL-13, IL-15, IL-17, IL-1Rα, IL-1β, IL-2, IL-2R, IL-4, IL-5, IL-6, IL-7, IL-8, IP-10, MCP-1, MIG, MIP-1α, MIP-1β, RANTES, TNF-α, or VEGF. In some embodiments, the increase in cytokine or chemokine expression is a local increase (i.e., specific to the CSF).

Also disclosed herein are methods of sustaining for at least about five days an increased number of T cells, compared to a baseline number, observed in a cerebrospinal fluid (CSF) of a patient diagnosed with a malignancy of a central nervous system, comprising infusing an effective amount of T cells into a CSF of a patient diagnosed with a malignancy of a central nervous system, in which an increased number of T cells observed, compared to a baseline number observed prior to the infusion step, is sustained for at least about five days.

In some embodiments, an effective amount of T cells (or T cells expressing a cell surface receptor that targets the tumor antigen) ranges from about 1×10⁶ cells to about 100×10⁶ cells, and in some embodiments, an effective amount of T cells ranges from about 2×10⁶ cells to about 50×10⁶ cells.

In some embodiments, the increased number of T cells observed is sustained for at least about six days, or the number of T cells observed does not return to the baseline number for about seven days.

In some embodiments, the T cells observed include infused T cells (e.g. CAR-expressing T cells), and in some embodiments, the T cells observed include endogenous T cells.

Also disclosed herein are methods of increasing a number of T cells in the cerebrospinal fluid (CSF) of a patient comprising, administering a composition comprising an effective amount of T cells into the CSF of a patient with a malignancy of the central nervous system, wherein the number of T cells detectable in the CSF is increased compared to pre-administration levels.

In some embodiments, the number of T cells detectable in the CSF is increased compared to pre-administration levels for up to seven days following administration. In some embodiments, the T cells detectable in the CSF comprise endogenous T cells and CAR-expressing T cells, and/or Type 1 T cells, and/or Type 2 T cells.

In some embodiments, the T cells detectable in the CSF comprise CD3+ T cells, and in some embodiments, the T cells detectable in the CSF comprise CD14+ CD11b+ HLA-DR+ mature myeloid populations. In some embodiments, CD19+ B cells and CD11b+αCD15+ granulocytes are detectable in the CSF following administration of the composition.

In some embodiments, reactive lymphocytes, monocytes, and macrophages are detectable in the CSF following administration of the composition.

Also disclosed herein are methods of determining the suitability of a patient with a malignancy for treatment with an IL-13Rα2-specific CAR T cell comprising, determining if a score attributed to a sample from the patient exhibits IL-13Rα2 expression above a predetermined threshold.

In some embodiments, the score attributed to the sample is calculated by determining the immunoreactivity of a resected tumor sample from a patient diagnosed with a malignancy by immunohistochemically staining the sample with a marker of IL-13Rα2, analyzing the strength of the staining, and calculating a score based on the strength of the staining, wherein a score that corresponds to moderate to strong staining intensity in the sample indicates that treatment with an IL-13Rα2-specific CAR T cell is suitable for the patient. In some embodiments, the score comprises counting the number of cells that have a weak, moderate, or strong staining intensity and assigning each intensity a weight (The H score, a method of quantitating immunohistochemical results, is based on the following formula: (3×the percentage of strongly staining cells)+(2× the percentage of moderately staining cells)+(1×the percentage of weakly staining cells), resulting in a range of 0 to 300). In some cases, the patient has a H score that is: greater that 50, 50-100, greater than 100, 100-200, greater than 200, 100-300, or greater than 250 for the relevant tumor-associated antigen. In some embodiments, an expression of Ki67 in the sample is also determined by immunohistochemical staining.

Also disclosed herein are methods of treating a patient with a malignancy comprising, administering to a patient diagnosed with a malignancy a composition comprising an effective dose of IL-13Rα2-specific CAR T cells, wherein the patient expresses IL-13Rα2 above a predetermined threshold.

In some embodiments, the predetermined threshold of IL-13Rα2 expression was previously identified as being suitable for a treatment comprising IL-13Rα2-specific CAR T cell therapy.

TIL are tumor infiltrating lymphocytes that can be isolated from a patient or a donor, expand ex vivo and re-infused into the patient in need thereof.

CAR T cells express chimeric T cell receptors that comprise an extracellular domain, a transmembrane region and an intracellular signaling domain. The extracellular domain includes a portion that binds the targeted cell and, optionally, a spacer, comprising, for example a portion human Fc domain. The transmembrane portion includes suitable transmembrane domain, for example, a CD4 transmembrane domain, a CD8 transmembrane domain, a CD28 transmembrane domain, a CD3 transmembrane domain or a 4IBB transmembrane domain. The intracellular signaling domain includes the signaling domain from the zeta chain of the human CD3 complex (CD3ζ) and one or more costimulatory domains, e.g., a 4-1BB costimulatory domain. The target cell binding portion of extracellular domain (for example a scFv or an ligand) enables the CAR, when expressed on the surface of a T cell, to direct T cell activity to those cells expressing the targeted cell surface molecule, for example HER2 or IL13Rα2, a receptor expressed on the surface of tumor cells, including malignant glioma cells.

A variety of different T cells, for example, patient-specific, autologous T cells, can be engineered to express a TCR or a CAR. Various T cell subsets can be used. In addition, CAR can be expressed in other immune cells such as NK cells. Where a patient is treated with an immune cell expressing a CAR or TCR the cell can be an autologous or allogenic T cell. In some cases, the cells used are CD4+ and CD8+ central memory T cells (T_{CM}), which are CD45RO+ CD62L+, and the use of such cells can improve long-term persistence of the cells after adoptive transfer compared to the use of other types of patient-specific T cells. The T_{CM} cells can include CD4+ cells and CD8+ cells.

Among the CAR useful in the methods described herein are those that target IL13Rα2. Such CAR can include IL13 having an amino acid modification, such as an E13Y mutation, that increases binding specificity.

The T cells used in the methods described herein can contain a nucleic acid molecule encoding a chimeric antigen receptor (CAR), wherein the chimeric antigen receptor comprises: human IL-13 or a variant thereof having 1-10 (e.g., 1 or 2) amino acid modifications; a transmembrane domain selected from: a CD4 transmembrane domain or variant thereof having 1-10 (e.g., 1 or 2) amino acid modifications, a CD8 transmembrane domain or variant thereof having 1-10 (e.g., 1 or 2) amino acid modifications, a CD28 transmembrane domain or a variant thereof having 1-10 (e.g., 1 or 2) amino acid modifications, and a CD3ζ transmembrane domain or a variant thereof having 1-10 (e.g., 1 or 2) amino acid modifications; a costimulatory domain; and CD3ζ signaling domain of a variant thereof having 1-10 (e.g., 1 or 2) amino acid modifications.

The inclusion of a costimulatory domain, such as the 4-1BB (CD137) or CD28 costimulatory domain in series with CD3ζ in the intracellular region enables the T cell to receive co-stimulatory signals. Thus, in various embodiments, the costimulatory domain is selected from the group consisting of: a CD28 costimulatory domain or a variant thereof having 1-10 (e.g., 1 or 2) amino acid modifications, a 4-IBB costimulatory domain or a variant thereof having 1-10 (e.g., 1 or 2) amino acid modifications and an OX40 costimulatory domain or a variant thereof having 1-10 (e.g., 1 or 2) amino acid modifications. In certain embodiments, a 4IBB costimulatory domain or a variant thereof having 1-10 (e.g., 1 or 2) amino acid modifications is present.

In additional embodiments of the methods, the CAR expressed by the T cells comprises: a variant of a human IL13 having 1-10 amino acid modification that increase binding specificity for IL13Rα2 versus IL13Rα1; the human IL-13 or variant thereof is an IL-13 variant comprising the amino acid sequence of SEQ ID NO:3 with 1 to 5 amino acid modifications, provided that the amino acid at position 11 of SEQ ID NO:3 other than E; two different costimulatory domains selected from the group consisting of: a CD28 costimulatory domain or a variant thereof having 1-10 (e.g., 1 or 2) amino acid modifications, a 4IBB costimulatory domain or a variant thereof having 1-10 (e.g., 1 or 2) amino acid modifications and an OX40 costimulatory domain or a variant thereof having 1-10 (e.g., 1 or 2) amino acid modifications; two different costimulatory domains selected from the group consisting of: a CD28 costimulatory domain or a variant thereof having 1-2 amino acid modifications, a 4IBB costimulatory domain or a variant thereof having 1-2 amino acid modifications and an OX40 costimulatory domain or a variant thereof having 1-2 amino acid modifications; human IL-13 or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ζ transmembrane domain or a variant thereof having 1-2 amino acid modifications; a costimulatory domain; and CD3ζ signaling domain of a variant thereof having 1-2 amino acid modifications; a spacer region located between the IL-13 or variant thereof and the transmembrane domain (e.g., the spacer region comprises an amino acid sequence selected from the group consisting of SEQ ID NO: 4, 14-20, 50 and 52); the spacer comprises an IgG hinge region; the spacer region comprises 10-150 amino acids; the 4-1BB signaling domain comprises the amino acid sequence of SEQ ID NO:6; the CD3ζ signaling domain comprises the amino acid sequence of SEQ ID NO:7; and a linker of 3 to 15 amino acids that is located between the costimulatory domain and the CD3ζ signaling domain or variant thereof. In certain embodiments where there are two costimulatory domains, one is an 4-IBB costimulatory domain and the other a costimulatory domain selected from: CD28 and CD28gg

In some embodiments of the methods described herein the T cells harbor a nucleic acid molecule that expresses a polypeptide comprising an amino acid sequence selected from SEQ ID NOs: 10 and 31-48; the chimeric antigen receptor comprises a IL-13/IgG4/CD4t/41-BB region comprising the amino acid of SEQ ID NO:11 and a CD3ζ signaling domain comprising the amino acid sequence of SEQ ID NO:7; and the chimeric antigen receptor comprises the amino acid sequence of SEQ ID NOs: 10 and 31-48.

Also disclosed are methods comprising intraventricular administration of a population of human T cells transduced by a vector comprising an expression cassette encoding a chimeric antigen receptor, wherein chimeric antigen receptor comprises: human IL-13 or a variant thereof having 1-10 amino acid modifications; a transmembrane domain selected from: a CD4 transmembrane domain or variant thereof having 1-10 amino acid modifications, a CD8 transmembrane domain or variant thereof having 1-10 amino acid modifications, a CD28 transmembrane domain or a variant thereof having 1-10 amino acid modifications, and a CD3ζ transmembrane domain or a variant thereof having 1-10 amino acid modifications; a costimulatory domain; and CD3ζ signaling domain of a variant thereof having 1-10 amino acid modifications. In various embodiments: the population of human T cells comprise a vector expressing a chimeric antigen receptor comprising an amino acid sequence selected from SEQ ID NOs: 10 and 31-48; the population of human T cells are comprises of central memory T cells (T_{CM} cells) (e.g., at least 20%, 30%, 40%, 50% 60%, 70%, 80% of the cells are T_{CM} cells; at least 10%, 15%, 20%, 25%, 30% or 35% of the T cells or the T_{CM} cells are CD4+ and at least 10%, 15%, 20%, 25%, 30% or 35% of the T cells or the T_{CM} cells are CD8+ cells).

Also described is a method of treating cancer in a patient comprising CNSadministration of a population of autologous or allogeneic human T cells (e.g., autologous or allogeneic T cells comprising T_{CM} cells, e.g., at least 20%, 30%, 40%, 50% 60%, 70%, 80% of the cells are T_{CM} cells; at least 15%, 20%, 25%, 30%, 35% of the T_{CM} cells are CD4+ and at least 15%, 20%, 25%, 30%, 35% of the T_{CM} cells are CD8+ cells) transduced by a vector comprising an expression cassette encoding a chimeric antigen receptor, wherein chimeric antigen receptor comprises an amino acid sequence selected from SEQ ID NOs: 10 and 31-48. In various embodiments: the population of human T cells comprise central memory T cells; the cancer is glioblastoma; and the transduced human T cells where prepared by a method comprising obtaining T cells from the patient, treating the T cells to isolate central memory T cells, and transducing at least a portion of the central memory cells to with a viral vector comprising an expression cassette encoding a chimeric antigen receptor, wherein chimeric antigen receptor comprises an amino acid sequence selected from SEQ ID NOs: 10 and 31-48.

Also described are method is which the T cells administered to the patient harbor a nucleic acid molecule encoding an polypeptide comprising an amino acid sequence that is at least 95% identical to an amino acid sequence selected from and SEQ ID NOs: 10 and 31-48; a nucleic acid molecule encoding an polypeptide comprising an amino acid sequence that is identical to an amino acid sequence selected from SEQ ID NO: 10 and 31-48 except for the presence of no more than 5 amino acid substitutions, deletions or insertions; a nucleic acid molecule encoding an polypeptide comprising an amino acid sequence that is identical to an amino acid sequence selected from SEQ ID NO:10 and SEQ ID NOs: 10 and 31-48 except for the presence of no more than 5 amino acid substitutions; and a nucleic acid molecule encoding an polypeptide comprising an amino acid sequence that is identical to an amino acid sequence selected from SEQ ID NO:10 and SEQ ID NOs: 10 and 31-48 except for the presence of no more than 2 amino acid substitutions.

Described herein are method for treating a patient by CSF administration of T cells harboring a nucleic acid molecule encoding a chimeric antigen receptor (CAR), wherein the chimeric antigen receptor comprises an scFv targeted to HER2 (e.g., comprises the amino acid sequence DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIKGSTSGGGSGGGSG GGGSSEVQLVESGGGLVQPGGSLRLSCAASGFNIKDTYIHWVRQAPGKGLEWVARIYPT NGYTRYADSVKGRFTISADTSKNTAYLQMNSLRAEDTAVYYCSRWGGDGFYAMDYW GQGTLVTVSS; SEQ ID NO: 49) or a variant thereof having 1-10 (e.g., 1 or 2) amino acid modifications; a spacer region; a transmembrane domain selected from: a CD4 transmembrane domain or variant thereof having 1-10 (e.g., 1 or 2) amino acid modifications, a CD8 transmembrane domain or variant thereof having 1-10 (e.g., 1 or 2) amino acid modifications, a CD28 transmembrane domain or a variant thereof having 1-10 (e.g., 1 or 2) amino acid modifications, and a CD3ζ transmembrane domain or a variant thereof having 1-10 (e.g., 1 or 2) amino acid modifications; a costimulatory domain; and CD3ζ signaling domain of a variant thereof having 1-10 (e.g., 1 or 2) amino acid modifications.

In various embodiments the costimulatory domain is selected from the group consisting of: a CD28 costimulatory domain or a variant thereof having 1-10 (e.g., 1 or 2) amino acid modifications, a 4-IBB costimulatory domain or a variant thereof having 1-10 (e.g., 1 or 2) amino acid modifications and an OX40 costimulatory domain or a variant thereof having 1-10 (e.g., 1 or 2) amino acid modifications. In certain embodiments, a 4IBB costimulatory domain or a variant thereof having 1-10 (e.g., 1 or 2) amino acid modifications in present.

In additional embodiments T cells administered to the patient express a CAR that comprises: an scFv targeted to HER2 (e.g., a humanized scFv); two different costimulatory domains selected from the group consisting of: a CD28 costimulatory domain or a variant thereof having 1-10 (e.g., 1 or 2) amino acid modifications, a 4IBB costimulatory domain or a variant thereof having 1-10 (e.g., 1 or 2) amino acid modifications and an OX40 costimulatory domain or a variant thereof having 1-10 (e.g., 1 or 2) amino acid modifications; two different costimulatory domains selected from the group consisting of: a CD28 costimulatory domain or a variant thereof having 1-2 amino acid modifications, a 4IBB costimulatory domain or a variant thereof having 1-2 amino acid modifications and an OX40 costimulatory domain or a variant thereof having 1-2 amino acid modifications; human IL-13 or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ζ transmembrane domain or a variant thereof having 1-2 amino acid modifications; a costimulatory domain; and CD3ζ signaling domain of a variant thereof having 1-2 amino acid modifications; a spacer region located between the IL-13 or variant thereof and the transmembrane domain (e.g., the spacer region comprises an amino acid sequence selected from the group consisting of SEQ ID NO: 4, 14-20, 50 and 52); the spacer comprises an IgG hinge region; the spacer region comprises 10-150 amino acids; the 4-1BB signaling domain comprises the amino acid sequence of SEQ ID NO:6; the CD3ζ signaling domain comprises the amino acid sequence of SEQ ID NO:7; and a linker of 3 to 15 amino acids that is located between the costimulatory domain and the CD3ζ signaling domain or variant thereof. In certain embodiments where there are two costimulatory domains, one is an 4-IBB costimulatory domain and the other a costimulatory domain selected from: CD28 and CD28gg

In some embodiments the T cells administered the patient harbor a nucleic acid molecule that expresses a polypeptide comprising an amino acid sequence selected from SEQ ID NOs: 53-56.

Also disclosed are methods comprising intraventricular administration of a population of human T cells transduced by a vector comprising an expression cassette encoding a chimeric antigen receptor comprising: an scFv targeted to HER2; a transmembrane domain selected from: a CD4 transmembrane domain or variant thereof having 1-10 amino acid modifications, a CD8 transmembrane domain or variant thereof having 1-10 amino acid modifications, a CD28 transmembrane domain or a variant thereof having 1-10 amino acid modifications, and a CD3ζ transmembrane domain or a variant thereof having 1-10 amino acid modifications; a costimulatory domain; and CD3ζ signaling domain of a variant thereof having 1-10 amino acid modifications. In various embodiments: the population of human T cells comprise a vector expressing a chimeric antigen receptor comprising an amino acid sequence selected from: SEQ ID NOs: 53-56; the population of human T cells are comprises of central memory T cells (T_{CM} cells) (e.g., at least 20%, 30%, 40%, 50% 60%, 70%, 80% of the cells are T_{CM} cells; at least 10%, 15%, 20%, 25%, 30%, 35% of the T cells or T_{CM} cells are CD4+ and/or at least 10%, 15%, 20%, 25%, 30%, 35% of the T cells or T_{CM} cells are CD8+ cells).

Also described is a method of treating cancer in a patient comprising intraventricular administration of a population of autologous or allogeneic human T cells (e.g., autologous or allogenic T cells comprising T_{CM} cells, e.g., at least 20%, 30%, 40%, 50% 60%, 70%, 80% of the cells are T_{CM} cells; at least 15%, 20%, 25%, 30%, 35% of the T_{CM} cells are CD4+ and/or at least 15%, 20%, 25%, 30%, 35% of the T_{CM} cells are CD8+ cells) transduced by a vector comprising an expression cassette encoding a chimeric antigen receptor, wherein chimeric antigen receptor comprises an amino acid sequence selected from SEQ ID NOs: 53-56. In various embodiments: the population of human T cells comprise central memory T cells; the cancer is glioblastoma; and the transduced human T cells where prepared by a method comprising obtaining T cells from the patient, treating the T cells to isolate central memory T cells, and transducing at least a portion of the central memory cells to with a viral vector comprising an expression cassette encoding a chimeric antigen receptor, wherein chimeric antigen receptor comprises an amino acid sequence selected from SEQ ID NOs: 53-56.

Also described is a method of treating cancer in a patient comprising intraventricular administration of a population of autologous or allogeneic human T cells (e.g., autologous or allogenic T cells comprising T_{CM} cells harboring: a nucleic acid molecule encoding an polypeptide comprising an amino acid sequence that is at least 95% identical to an amino acid sequence selected from SEQ ID NOs: 53-56; a nucleic acid molecule encoding an polypeptide comprising an amino acid sequence that is identical to an amino acid sequence selected from SEQ ID NOs: 53-56 except for the presence of no more than 5 amino acid substitutions, deletions or insertions; a nucleic acid molecule encoding an polypeptide comprising an amino acid sequence that is identical to an amino acid sequence selected from SEQ ID NOs: 53-56 except for the presence of no more than 5 amino acid substitutions; and a nucleic acid molecule encoding an polypeptide comprising an amino acid sequence that is identical to an amino acid sequence selected from SEQ ID NOs: 53-56, except for the presence of no more than 2 amino acid substitutions.

Certain CAR described herein, for example, the IL13(EQ)BBζ CAR and the IL13(EQ)CD28- BBζ CAR, have certain beneficial characteristics compared to certain other IL13-targeted CAR. For example, they have improved selectivity for IL13Rα, elicit lower Th2 cytokine production, particularly lower IL13 production.

T cells expressing a CAR targeting IL13Rα2 can be useful in treatment of cancers such as glioblastoma, as well as other cancers that expresses IL13RαL2. Thus, this disclosure includes methods for treating cancer using T cells expressing a CAR described herein.

T cells expressing a CAR targeting HER2 can be useful in treatment of cancers such as glioblastoma, as well as other cancer that expresses HER2, for example breast cancer that has spread to the central nervous system. Thus, this disclosure includes methods for treating cancer using T cells expressing a CAR described herein.

The CAR described herein can include a spacer region located between the targeting domain and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 14) |
| IgG4 hinge (S→P) | 12 aa | ESKYGPPCPPCP (SEQ ID NO: 15) |
| (S228P) | | |
| IgG4 hinge | 12 aa | ESKYGPPCPSCP (SEQ ID NO: 52) |
| IgG4 hinge + linker | 22 aa | ESKYGPPCPPCPGGGSSGGGSG (SEQ ID NO: 16) |
| CD28 hinge | 39 aa | IEVMYPPPYLDNEKSNGTIIHVKGKHLCPSPLFPGPS |
| | | KP (SEQ ID NO: 17) |
| CD8 hinge-48 aa | 48 aa | AKPTTTPAPRPPTPAPTIASQPLSLRPEACRPAAGGA |
| | | VHTRGLDFACD (SEQ ID NO: 18) |
| CD8 hinge-45 aa | 45 aa | TTTPAPRPPTPAPTIASQPLSLRPEACRPAAGGAVHT |
| | | RGLDFACD (SEQ ID NO: 19) |
| IgG4(HL-CH3) | 129 aa | ESKYGPPCPPCPGGGSSGGGSGGQPREPQVYTLPPS |
| | | QEEMTKNQVSLTCLVKGFYPSDIAVEWESNGQPEN |
| | | NYKTTPPVLDSDGSFFLYSRLTVDKSRWQEGNVFS |
| | | CSVMHEALHNHYTQKSLSLSLGK (SEQ ID NO: 20) |
| IgG4(L235E, N297Q) | 229 aa | ESKYGPPCPSCPAPEFEGGPSVFLFPPKPKDTLMISR |
| | | TPEVTCVVVDVSQEDPEVQFNWYVDGVEVHQAKT |
| | | KPREEQFNSTYRVVSVLTVLHQDWLNGKEYKCKV |
| | | SNKGLPSSIEKTISKAKGQPREPQVYTLPPSQEEMT |
| | | KNQVSLTCLVKGFYPSDIAVEWESNGQPENNYKTT |
| | | PPVLDSDGSFFLYSRLTVDKSRWQEGNVFSCSVMH |
| | | EALHNHYTQKSLSLSLGK (SEQ ID NO: 4) |
| IgG4(S228P, L235E, N297Q) | 229 aa | ESKYGPPCPPCPAPEFEGGPSVFLFPPKPKDTLMISR |
| | | TPEVTCVVVDVSQEDPEVQFNWYVDGVEVHQAKT |
| | | KPREEQFNSTYRVVSVLTVLHQDWLNGKEYKCKV |
| | | SNKGLPSSIEKTISKAKGQPREPQVYTLPPSQEEMT |
| | | KNQVSLTCLVKGFYPSDIAVEWESNGQPENNYKTT |
| | | PPVLDSDGSFFLYSRLTVDKSRWQEGNVFSCSVMH |
| | | EALHNHYTQKSLSLSLGK (SEQ ID NO: 51) |
| IgG4(CH3) | 107 aa | GQPREPQVYTLPPSQEEMTKNQVSLTCLVKGFYPS |
| | | DIAVEWESNGQPENNYKTTPPVLDSDGSFFLYSRLT |
| | | VDKSRWQEGNVFSCSVMHEALHNHYTQKSLSLSL |
| | | GK (SEQ ID NO: 50) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one ore more amino acid modifications, for example, 1, 2, 3, 4 or 5 substitutions, e.g., substitutions that reduce off-target binding.

An “amino acid modification” refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

A variety of transmembrane domains can be used in CAR expressed by the cells used in the methods described herein. Table 2 includes examples of suitable transmembrane domains. Where a spacer region is present, the transmembrane domain is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 21) |
| CD28 | NM_006139 | 27 aa | FWVLVVVGGVLACYSLLVTVAF |
| | | | IIFWV (SEQ ID NO: 22) |
| CD28(M) | NM_006139 | 28 aa | MFWVLVVVGGVLACYSLLVTVA |
| | | | FIIFWV (SEQ ID NO: 22) |
| CD4 | M35160 | 22 aa | MALIVLGGVAGLLLFIGLGIFF |
| | | | (SEQ ID NO: 5) |
| CD8tm | NM_001768 | 21 aa | IYIWAPLAGTCGVLLLSLVIT |
| | | | (SEQ ID NO: 23) |
| CD8tm2 | NM_001768 | 23 aa | IYIWAPLAGTCGVLLLSLVIT |
| | | | LY (SEQ ID NO: 24) |
| CD8tm3 | NM_001768 | 24 aa | IYIWAPLAGTCGVLLLSLVITL |
| | | | YC (SEQ ID NO: 25) |
| 41BB | NM_001561 | 27 aa | IISFFLALTSTALLFLLFFLTL |
| | | | RFSVV (SEQ ID NO: 26) |

Many of the CAR expressed by the cells used in the methods described herein include one or more (e.g., two) costimulatory domains. The costimulatory domain(s) are located between the transmembrane domain and the CD3 signaling domain. Table 3 includes examples of suitable costimulatory domains together with the sequence of the CD3 signaling domain.

TABLE 3
Examples of Costimulatory Domains

| Name | Accession | Length | Sequence |
|---|---|---|---|
| CD3ζ | J04132.1 | 113 aa | RVKFSRSADAPAYQQGQNQLYNELNLGRREEYD |
| | | | VLDKRRGRDPEMGGKPRRKNPQEGLYNELQKDK |
| | | | MAEAYSEIGMKGERRRGKGHDGLYQGLSTATKD |
| | | | TYDALHMQALPPR (SEQ ID NO: 7) |
| CD28 | NM_006139 | 42 aa | RSKRSRLLHSDYMNMTPRRPGPTRKHYQPYAPPR |
| | | | DFAAYRS (SEQ ID NO: 27) |
| CD28gg* | NM_006139 | 42 aa | RSKRSRGGHSDYMNMTPRRPGPTRKHYQPYAPP |
| | | | RDFAAYRS (SEQ ID NO: 28) |
| 41BB | NM_001561 | 42 aa | KRGRKKLLYIFKQPFMRPVQTTQEEDGCSCRFPEE |
| | | | EEGGCEL (SEQ ID NO: 29) |
| OX40 | | 42 aa | ALYLLRRDQRLPPDAHKPPGGGSFRTPIQEEQADA |
| | | | HSTLAKI (SEQ ID NO: 30) |

### DESCRIPTION OF DRAWINGS

FIG. 1 is a schematic depiction of IL13(E13Y)-zetakine CAR (Left) composed of the IL13Rα2-specific human IL-13 variant (huIL-13(E13Y)), human IgG4 Fc spacer (huγ₄F_{c}), human CD4 transmembrane (huCD4 tm), and human CD3ζ chain cytoplasmic (huCD3ζ cyt) portions as indicated. Also depicted is a IL13(EQ)BBζ CAR which is the same as the IL13(E13Y)-zetakine with the exception of the two point mutations, L235E and N297Q indicated in red, that are located in the CH2 domain of the IgG4 spacer, and the addition of a costimulatory 4-1BB cytoplasmic domain (4-1BB cyt).

FIGS. 2A-C depict certain vectors and open reading frames. A is a diagram of the cDNA open reading frame of the 2670 nucleotide IL13(EQ)BBZ-T2ACD19t construct, where the IL13Rα2-specific ligand IL13(E13Y), IgG4(EQ) Fc hinge, CD4 transmembrane, 4-1BB cytoplasmic signaling, three-glycine linker, and CD3ζ cytoplasmic signaling domains of the IL13(EQ)BBZ CAR, as well as the T2A ribosome skip and truncated CD19 sequences are indicated. The human GM-CSF receptor alpha and CD19 signal sequences that drive surface expression of the IL13(EQ)BBζ CAR and CD19t are also indicated. B is a diagram of the sequences flanked by long terminal repeats (indicated by ‘R’) that will integrate into the host genome. C is a map of the IL13(EQ)BBZ-T2A-CD19t_epHIV7 plasmid.

FIG. 3 depicts the construction of pHIV7.

FIG. 4 depicts the elements of pHIV7.

FIG. 5 depicts a production scheme for IL13(EQ)BBζ/CD19t+ T_{CM}.

FIGS. 6A-C depicts the results of flow cytometric analysis of surface transgene and T cell marker expression. IL13(EQ)BBζ/CD19t+ T_{CM} HD006.5 and HD187.1 were co-stained with anti-IL13-PE and anti-CD8-FITC to detect CD8+ CAR+ and CD4+ (i.e., CD8 negative) CAR+ cells (A), or anti-CD19-PE and anti-CD4-FITC to detect CD4+ CD19t+ and CD8+(i.e., CD4 negative) CAR+ cells (B). IL13(EQ)BBζ/CD19t+ T_{CM} HD006.5 and HD187.1 stained with fluorochromeconjugatedanti-CD3, TCR, CD4, CD8, CD62L and CD28 (grey histograms) or isotype controls (black histograms) (C). In all cases the percentages based on viable lymphocytes (DAPI negative) stained above isotype.

FIGS. 7A-D depict the results of experiments comparing route of CAR+ T cell delivery (i.e. versus i.v.) for large established tumors. EGFP-ffLuc+ PBT030-2 TSs (1×10⁵) were implanted into the right forebrain of NSG mice. On days 19 and 26, mice were injected i.v. through the tail vein with either 5×10⁶ CAR+ IL13(EQ)BBζ+ TCM (11.8×10⁶ total cells; n=4), or mock TCM (11.8×10⁶ cells; n=4). Alternatively, on days 19, 22, 26 and 29 mice were injected i.e. with either 1×10⁶ CAR+ IL13(EQ)BBζ+ TCM (2.4×10⁶ total cells; n=4), or mock TCM (2.4×10⁶ cells; n=5). Average ffLuc flux (photons/sec) over time shows that i.e. delivered IL13(EQ)BBζ TCM mediates tumor regression of day 19 tumors. By comparison, i.v. delivered T cells do not shown reduction in tumor burden as compared to untreated or mock TCM controls (A). Kaplan Meier survival curve demonstrates improved survival for mice treated i.e. IL13(EQ)BBZ TCM as compared to mice treated with i.v. administered CAR+ TCM (p=0.0003 log rank test) (B). Representative H&E and CD3 IHC of mice treated i.v. (C) versus i.e. (D) with IL13(EQ)BBZ+ TCM. CD3+ T cells were only detected in the i.e. treated group, with no CD3+ cells detected in the tumor or surrounding brain parenchyma for i.v. treated mice.

FIGS. 8A-B depict the results of studies showing that CAR+ T cell injected intracranially, either intratumoral (i.e.t.) or intraventricular (i.e.v.), can traffic to tumors on the opposite hemisphere. EGFP-ffLuc+ PBT030-2 TSs (1×10¹) were stereotactically implanted into the right and left forebrains of NSG mice. On day 6, mice were injected i.e. at the right tumor site with 1.0×10⁶ IL13(EQ)BBζ+ TCM (1.6×10⁶ total cells; 63% CAR; n=4). Schematic of multifocal glioma experimental model (A). CD3 IHC showing T cells infiltrating both the right and left tumor sites (B).

FIGS. 9A-9B depict the amino acid sequence of IL13(EQ)BBζ/CD19t+ (SEQ ID NO:10).

FIGS. 10A-10O depict a sequence comparison of IL13(EQ)41BBζ[IL13{EQ}41BBζ T2A-CD19t_epHIV7; pF02630] (SEQ ID NO:12) and CD19Rop_epHIV7 (pJ01683) (SEQ ID NO:13).

FIG. 11 depicts the amino acid sequence of IL13(EmY)-CD8h3-CD8tm2-41BB Zeta (SEQ ID NO:31 with GMCSFRa signal peptide; SEQ ID NO:39 without GMCSFRa signal peptide).

FIG. 12 depicts the amino acid sequence of IL13(EmY)-CD8h3-CD28tm-CD28gg-41BB-Zeta (SEQ ID NO:32 with GMCSFRa signal peptide; SEQ ID NO:40 without GMSCFRa signal peptide).

FIG. 13 depicts the amino acid sequence of IL13(EmY)-IgG4(HL-CH3)-CD4tm-41BB-Zeta (SEQ ID NO:33 with GMCSFRa signal peptide; SEQ ID NO:41 without GMCSFRa signal peptide).

FIG. 14 depicts the amino acid sequence of IL13(EmY)-IgG4(L235E,N297Q)-CD8tm-41BB-Zeta (SEQ ID NO:34 with GMCSFRa signal peptide; SEQ ID NO:42 without GMCSFRa signal peptide).

FIG. 15 depicts the amino acid sequence of IL13(EmY)-Linker-CD28tm-CD28gg-41BB-Zeta (SEQ ID NO:35 with GMCSFRa signal peptide; SEQ ID NO:43 without GMCSFRa signal peptide).

FIG. 16 depicts the amino acid sequence of IL13(EmY)-HL-CD28m-CD28gg-41BB-Zeta (SEQ ID NO:36 with GMCSFRa signal peptide; SEQ ID NO:44 without GMSCFRa signal peptide).

FIG. 17 depicts the amino acid sequence of IL13(EmY)-IgG4(HL-CH3)-CD28tm-CD28gg-41BB-Zeta (SEQ ID NO:37 with GMSCFRa signal peptide; SEQ ID NO:45 without GMCSFRa signal peptide).

FIG. 18 depicts the amino acid sequence of IL13(EmY) IgG4(L235E,N297Q)-CD28tm-CD28gg-41BB-Zeta (SEQ ID NO:38 with GMCSFRa signal peptide; SEQ ID NO:46 without GMCSFRa signal peptide).

FIG. 19 depicts the amino acid sequence of IL13(EmY)-CD8h3-CD8tm-41BB Zeta (SEQ ID NO:47 with GMCSFRa signal peptide; SEQ ID NO:48 without GMCSFRa signal peptide).

FIG. 20 depicts the amino acid sequence of Her2scFv-IgG4(L235E, N297Q)-CD28tm-CD28gg-Zeta-T2A-CD19t (SEQ ID NO:53). The various domains are listed in order below the sequence and are indicated by alternating underlining and non-underlining. The mature CAR sequence (SEQ ID NO:54) does not include the GMCSFRa signal peptide, theT2A skip sequence or truncated CD19.

FIG. 21 depicts the amino acid sequence of Her2scFv-IgG4(L235E,N297Q)-CD8tm-41BB-Zeta-T2A-CD19t (SEQ ID NO:55). The various domains are listed in order below the sequence and are indicated by alternating underlining and non-underlining. The mature CAR sequence (SEQ ID NO:56) does not include the GMCSFRa signal peptide, theT2A skip sequence or truncated CD19.

FIGS. 22A-D depict HER2-specific CAR constructs and CAR T cell expansion data.

FIGS. 23A-D depict in vitro characterization of HER2-CAR T cells against breast cancer cell lines.

FIGS. 24A-F depict the result of studies on the in vitro tumor activity of HER2-CAR T cells.

FIGS. 25A-I depict the result of studies on the in vivo anti-tumor efficacy of local intratumorally-delivered HER2-CAR T cells.

FIGS. 26A-D depict the results of studies on local delivery of HER2-CAR T cells in human orthotopic BBM xenograft models.

FIGS. 27A-D depict the results of studies on intraventricular delivery of HER2-CAR T cells.

FIG. 28 schematically depicts the locations of tumor cell injection and CAR T cell injection for a study of intratumoral and intraventricular injection of CAR T cells targeting IL13a2R in a murine model of glioblastoma.

FIGS. 29A-C depict the results of studies demonstrating regression of established glioma tumor xenografts after administration of IL13(EQ)BBζ/CD19t+ T_{CM}. ffLuc⁺ PBT030-2 tumor cells (1×10⁵) were stereotactically implanted into the right and left forebrains of NSG mice. On day 6, mice were injected either ict or icy with 1×10⁶ IL13(EQ)BBζ+ Tcm (1.6×10⁶ total cells; 63% CAR+) as described in FIG. 4.1 above. A, Representative mice from each group showing relative tumor burden using Xenogen Living Image. B, Average Xenogen flux of left and right brain hemispheres (region of interest, ROI) from the mice (n=4-5) of each group, where each successive bar represents day 5, 9, 12, 15, and 19, respectively. *, p<0.05 when compared to the respective ROI and day/bar of the untreated PBT030-2 group using an unpaired Student's t-test. C, Average Xenogen flux of the whole brain 13 days after T cell injection. *, p=0.0407 when comparing icv group to untreated PBT030-2 group using the unpaired Student's t-test. These data are representative of three separate multifocal GBM experiments.

FIG. 30 depicts the results of studies demonstrating that huCD3+ cells are detected in the right and left brain tumors/hemispheres after ict and icv administration of IL13(EQ)BBζ/CD19t+ T_{CM}. ffLuc⁺ PBT030-2 tumor cells (1×10⁵) were stereotactically implanted into the right and left forebrains of NSG mice. On day 6, mice were injected either ict (left images) or icy (right images) with 1×10⁶ IL13(EQ)BBζ+ Tcm (1.6×10⁶ total cells; 63% CAR+). One week (top images) and two weeks (bottom images) after T cell administration, 2-3 mice were euthanized from each group, brains were harvested, embedded in paraffin, and IHC was performed with anti-human CD3 antibody to detect T cells. Representative IHC images of the left and right tumor sites from mice of each group (ict: m406 and m410; icy: m414 and m415) are depicted. Inlays depict the xenogen flux images of the mice at the day of euthanasia and brain harvest.

FIG. 31 schematically depicts the time course of CAR T cell preparation and treatment for a clinical trial of CAR T cells for treatment of glioblastoma (A) and provides several dosing schemes (B).

FIG. 32 presents analysis of CAR T cell persistence, as monitored by CD19 (A) and presence of GBM cells as monitored by IL13Rα2 expression (B).

FIG. 33 presents imaging results from Patient UPN097 in the region of the catheter used for intratumoral administration.

FIG. 34 is a series of graphs showing the levels of various cytokines during the course of treatment for one patient.

FIGS. 35A-C are images depicting egression of recurrent multifocal glioblastoma, including spinal metastasis, following intraventricular delivery of IL13Rα2-targeted CAR T cells. A patient presenting with a recurrent multifocal GBM, including one metastatic site in the spine and extensive leptomeningeal disease was treated with six local infusions of IL13BBζ Tcm into the resection cavity of the largest recurrent tumor focus (1 cycle of 2 M, and 5 cycles of 10M CAR+ T cells). While the CAR T cell injection site remained stable without evidence of disease recurrence for over 7-weeks, other disease foci distant from the CAR T cell injection site continued to progress (data not shown). This patient then received five weekly intraventricular (icv) infusions of IL13BBζ Tcm (1 cycles of 2 M, and 4 cycles of 10M CAR+ T cells). Shown are MRI and/or PET images of (A) transverse brain section, (B) saggital brain section, and (C) transverse (top) and frontal (bottom) sections of the spine before (left) and one week after (right) completion of i.e.v. therapy, with tumor lesion sites indicated by red arrows in each image.

FIGS. 36A-B shows treatment regimens with enrollment on NCT02208362 and a compassionate use protocol. Enrollment on NCT02208362 was set at day 0 (A), with initiation of compassionate use protocol on day 107 (B). NovoTTF-100A, a portable medical device that delivers low intensity, intermediate frequency, alternating electric fields by means of noninvasive, disposable scalp electrodes; FGFR, fibroblast growth factor receptor; MRI, magnetic resonance imaging, all of which was performed on the brain unless otherwise indicated; ICT, intracavitary; PET, positron emission tomography, performed at the indicated sites; ICV, intracerebroventricular.

FIGS. 37A-C shows immunohistochemistry of primary and recurrent tumors. Tumor resected at initial diagnosis (A), and recurrent tumor (T1) resected at time of Rickham placement under NCT02208362 (B, C). Immunochemical staining using either IL13Rα2-specific or Ki67-specific DAB with hematoxylin counterstain are depicted, with red boxes outlining the successive magnified images going left to right.

FIGS. 38A-B shows tumor lesion identification. (A) Identifying characteristics of GBM lesions T1-T8. (B) Brain MRI scans depicting the sites of T1-T7.

FIGS. 39A-C shows resected tumor region remains stable, without evidence of disease progression/recurrence following intracavitary delivery of IL13BBζ TCM. (A) Flow cytometry analysis of the IL13BBζ TCM cell product. Top row, transduction with the indicated construct drove coordinate surface expression of the IL13BBζ CAR (detected with anti-IL13) and the CD19t marker (detected with anti-CD19) via the T2A ribosomal skip sequence. Staining profiles for the T cell markers TCR-α/β, CD3, CD4 and CD8, as well as the exhaustion markers LAG-3, TIM-3, KLRG1 and PD-1 are depicted in the middle row; staining profiles for the memory markers CD62L, CD45RO, CCR7, CD27, and CD28, as well as the naïve T cell marker CD45RA are depicted in the bottom row. (B) Treatment schema under NCT02208362. After the patient experienced recurrence and underwent tumor excision with placement of an intracavitary (ICT) Rickham catheter, 6 cycles of ICT cell doses (1 cycle of 2×10⁶, and 5 cycles of 10×10⁶ CAR+ cells) were administered with one week rest between cycles 3 and 4. Red arrow, site of IL13BBζ TCM delivery. (C) Yellow circles on brain MRI scans show the site of resected tumor where catheter was placed (T1), as well as the resected-only tumor sites in the frontal lobe (T2, T3), and the newly arising tumor sites (T6, T7) over the 51-day ICT treatment time period.

FIGS. 40A-E shows regression of recurrent multifocal glioblastoma, including spinal metastases, following intracerebroventricular delivery of IL13Rα2-targeted CAR T cells. (A) Treatment schema under compassionate use protocol. After the patient underwent placement of an intracerebroventricular (ICV) Rickham catheter, 5 cycles of ICV cell doses (1 cycle of 2×10⁶ and 4 cycles of 10×10⁶ CAR+ cells, indicated as cycles 7 through 11) were administered with one week rest between cycles 9 and 10. Red arrow, site of IL13BBζ TCM delivery. MRI and/or PET images of (B) transverse brain sections, (C) saggital brain sections, and (D) transverse (top) and frontal (bottom) sections of the spine before (left) and one week after (right) completion of ICV therapy, with tumor lesion sites indicated by yellow circles in each image. (E) Maximum lesion areas for non-resected tumors T4-T8 depict their respective decreases over time with ICV therapy.

FIG. 41 shows regression of recurrent multifocal GBM following ICV delivery of IL13Rα2-targeted CAR T cells. Maximum lesion areas for non-resected tumors T4-T7 are depicted.

FIG. 42A-C shows analysis of cell infiltrates and cytokines from cerebrospinal fluid (CSF) samples. (A) CSF cellular infiltrate numbers spiked after ICV cycles 9, 10 and 11, with flow cytometric evidence of CD19+ B cells, both CAR+ (i.e., CD19t+) and non-engineered CD3+ T cells, CD11b+ CD15+ granulocytes, and CD11b+ CD14+ HLA-DR+ monocytes. (B) Evaluation of the CD3+ T cell population in the CSF for the presence of gene-modified (i.e., CD19t+) T cells. CD3-gated cells from the CSF collected at the indicated day of cycles 9, 10 and 11 were co-stained for CD19 and CD8 (top histograms). Percentages of immunoreactive cells were then used to calculate numbers of total CD3+ T cells and CD19+ CD3+(IL13BBζ TCM) cells per mL of CSF fluid at each time point. (C) Fold change in cytokine levels with ICV treatment cycles 7-11. Only those cytokines from the 30-plex analysis that exhibited a 10-fold or more change compared to pre-treatment levels are depicted.

### DETAILED DESCRIPTION

Described below is the structure, construction and characterization of various CAR T cells and their use in treating cancers of the central nervous system. A chimeric antigen (CAR) is a recombinant biomolecule that contains, at a minimum, an extracellular recognition domain, a transmembrane region, and an intracellular signaling domain. The term “antigen,” therefore, is not limited to molecules that bind antibodies, but to any molecule that can bind specifically to a target. For example, a CAR can include a ligand that specifically binds a cell surface receptor. The extracellular recognition domain (also referred to as the extracellular domain or simply by the recognition element which it contains) comprises a recognition element that specifically binds to a molecule present on the cell surface of a target cell. The transmembrane region anchors the CAR in the membrane. The intracellular signaling domain comprises the signaling domain from the zeta chain of the human CD3 complex and optionally comprises one or more costimulatory signaling domains. CARs can both to bind antigen and transduce T cell activation, independent of MHC restriction. Thus, CARs are “universal” immunoreceptors which can treat a population of patients with antigen-positive tumors irrespective of their HLA genotype. Adoptive immunotherapy using T lymphocytes that express a tumor-specific CAR can be a powerful therapeutic strategy for the treatment of cancer.

In some cases the CAR described herein can be produced using a vector in which the CAR open reading frame is followed by a T2A ribosome skip sequence and a truncated CD19 (CD19t), which lacks the cytoplasmic signaling tail (truncated at amino acid 323). In this arrangement, co-expression of CD19t provides an inert, non-immunogenic surface marker that allows for accurate measurement of gene modified cells, and enables positive selection of gene-modified cells, as well as efficient cell tracking and/or imaging of the therapeutic T cells in vivo following adoptive transfer. Co-expression of CD19t provides a marker for immunological targeting of the transduced cells in vivo using clinically available antibodies and/or immunotoxin reagents to selectively delete the therapeutic cells, and thereby functioning as a suicide switch.

The disclosed methods of treatment using CAR T cells can be performed at various doses and across various timeframes. For example, a patient receiving an infusion, administration, or injection of CAR T cells (e.g. IL-13Rα2-specific CAR T cells) may receive a single dose comprising between 1×10⁶ and 15×10⁶ cells. In other words, a single dose for use in the disclosed methods can comprise 1×10⁶, 2×10⁶, 3×10⁶, 4×10⁶, 5×10⁶, 6×10⁶, 7×10⁶, 8×10⁶, 9×10⁶, 10×10⁶, 11×10⁶, 12×10⁶, 13×10⁶, 14×10⁶, or 15×10⁶ cells. Over the entire course of treatment, a patient may receive a cumulative or total dose of cells between 20×10⁶ and 150×10⁶ T cells. For instance, the patient may receive about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, about 100×10⁶, about 105×10⁶, about 110×10⁶, about 115×10⁶, about 120×10⁶, about 125×10⁶, about 130×10⁶, about 135×10⁶, about 140×10⁶, about 145×10⁶, or about 150×10⁶ or more T cells over the course of treatment. In some embodiments, a patient can receive a total dose of at least 90×10⁶ T cells. In one embodiment, a patient can receive a total dose of 94×10⁶ T cells.

Furthermore, the doses may be administered according to different regimens and timetables. For example, the disclosed methods can comprise an infusion, administration, or injection once a day, once every two days, once every three days, once every four days, once every five days, once every six days, a week, once every two weeks, once every three weeks, once every four weeks, once a month, once every other month, once every three months, or once every six months. In some embodiments, the disclosed methods can comprise continuous infusion, for instance, from a wearable pump. Similarly, the total time course of treatment may be about 5 weeks, about 10 weeks, about 15 weeks, about 20 weeks, about 25 weeks, about 30 weeks, about 35 weeks, about 40 weeks, about 45 weeks, about 50 weeks, about 55 weeks, about 60 weeks, about 65 weeks, about 70 weeks, about 75 weeks, or more. The patient may receive 1, 2, 3, 4, 5, 6, 7, 8, 9, 10, 11, 12, 13, 14, 15, 16, 17, 18, 19, or 20 or more infusions, administrations, or injections of T cells over the course of treatment according to the disclosed methods. For example, in one embodiment, a patient can receive 11 infusions of T cells over the course of 15 weeks.

Treating cancer, and more specifically gliomas like glioblastoma, according to the disclosed methods can result in numerous therapeutic effects. For instance, treatment with the disclosed CAR T cells can result in an increase in the level of cytokines and chemokines in the CSF of a patient being treated according to the disclosed methods. Cytokine and/or chemokine expression may increase by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100%, or cytokine and/or chemokine expression may increase by at least 1-fold, at least 2-fold, at least 3-fold, at least 4-fold, at least 5-fold, at least 6-fold, at least 7-fold, at least 8-fold, at least 9-fold, or at least 10-fold compared to baseline levels, as measured prior to treatment with a composition comprising CAR T cells. This increase in expression may be observed for 1, 2, 3, 4, 5, 6, 7, 8, 9, 10, 11, 12, 13, 14, or 15 or more cytokines or chemokines.

In particular, the expression of at least one of EGF, Eotaxin, FGF, G-CSF, GM-CSF, HGF, IFN-α, IFN-γ, IL-10, IL-12, IL-13, IL-15, IL-17, IL-1Rα, IL-1β, IL-2, IL-2R, IL-4, IL-5, IL-6, IL-7, IL-8, IP-10, MCP-1, MIG, MIP-1α, MIP-1β, RANTES, TNF-α, and VEGF may increase as a result of treatment with CAR T cells as disclosed herein. Furthermore, the increase in cytokine and/or chemokine expression may be local (i.e. the increase is only observable in the CNS and CFS, while serum levels of cytokines and chemokines remain unchanged.

Treatment according to the disclosed methods may also result in an increase in T cells detectable in the CSF. While at least some of the T cells detectable in the CSF following treatment will likely be CAR-expressing T cells, there may also be an increase in endogenous T cells that are recruited to the CSF. Although not wanting to be bound by theory, the increase in endogenous T cells may be a result of the recruitment of Type 1 and Type 2 T helper cells due to the increase in local cytokine levels. Additionally, the detectable T cells can comprise CD3+ T cells, as well as CD14+ CD11b+ HLA-DR+ mature myeloid populations, CD19+ B cells and CD11b+ CD15+ granulocytes, and/or reactive lymphocytes, monocytes, and macrophages.

The increase in the number of T cells in the CSF may be detectable for a specific period of time following treatment according to the disclosed methods. A detectable increase in T cells in the CSF may persist or be sustained for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days following administration of a composition comprising T cells. For example, an increase in the number of T cells observed in the CSF may not return to baseline levels (i.e. the number of T cells detectable prior to treatment) for about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. The number of T cells detectable in the CSF may increase by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100%, or by at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold compared to baseline levels, as measured prior to treatment with a composition comprising CAR T cells.

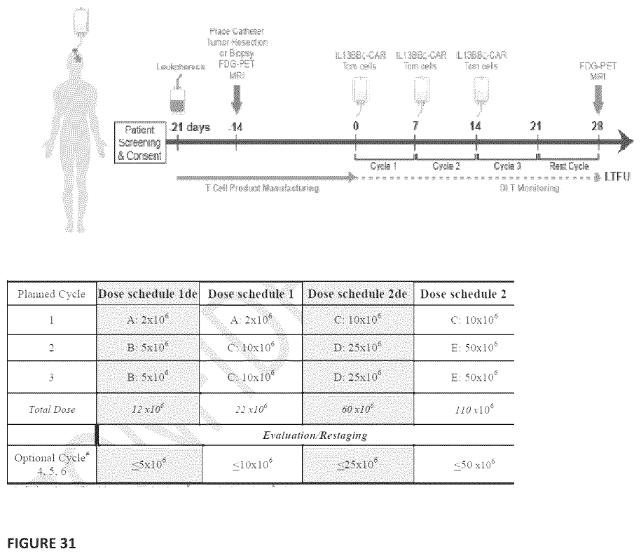

The time course of CAR T cell preparation and treatment is depicted in FIG. 31. Concurrent with the manufacturing process, research participants underwent resection of their tumor(s) followed by placement of a Rickham catheter and baseline imaging.

Patient UPN097 underwent tumor resection and was treated in Cycle 1 with 2×10⁶ cells and in Cycle 2 with 10×10⁶ cells. In both Cycle 1 and Cycle 2 the cells were administered to the cavity left by resection. After the second cycle Patient UPN097 was taken off the study due to rapid tumor progression.

Patient UPN109 was treated in Cycle 1 with 2×10⁶ cells and in Cycles 2 and 3 with 10×10⁶ cells. After a rest period, Patient UPN109 was treated in Cycles 4, 5 and 6 with 10×10⁶ cells. In Cycles 1-6 the cells were administered intratumorally. In Cycle 7 the patient was treated with 2×10⁶ cells. In Cycles 8 and 9 the patient was treated with 10×10⁶ cells. In Cycles 7-10 the administration was intraventricular.

As used herein, the term “intraventricular” refers to the space inside the ventricular system, specifically the cerebral ventricles. Accordingly, the term “intraventricular” and “intracerebroventricular” may be used interchangeably throughout this disclosure. Accordingly, “intraventricular administration” or “intraventricular injection” refer to delivery of a composition into the ventricals of the brain (i.e. the cerebral ventricles). The cerebral ventricles are a series of interconnected, fluid-filled spaces that lie in the core of the forebrain and brainstem. This system comprises four ventricles: the right and left lateral ventricles (one of which is found in each hemisphere of the brain), the third ventricle, and the fourth ventricle.

The disclosed methods comprise various routes of administering the compositions comprising T cells. For instance, in some embodiments, the disclosed compositions may be delivered or administered intraventricularly. In some embodiments, the disclosed compositions may be delivered or administered into the spinal canal (i.e. intrathecal delivery). In some embodiments, the disclosed compositions may be delivered or administered into the epidural space of the spinal cord (i.e. epidural delivery). In some embodiments, the disclosed compositions may be delivered or administered directly into a tumor (i.e. intratumoral delivery). In some embodiments, the disclosed compositions may be delivered or administered into a cavity formed by the resection of tumor tissue (i.e. intracavity delivery). Furthermore, in some embodiments, the disclosed methods can comprise a combination of the aforementioned routes of administration. For instance, a patient may receive at least one dose of the composition comprising T cells via intracavity delivery, followed by at least one dose of the composition via intraventricular delivery.

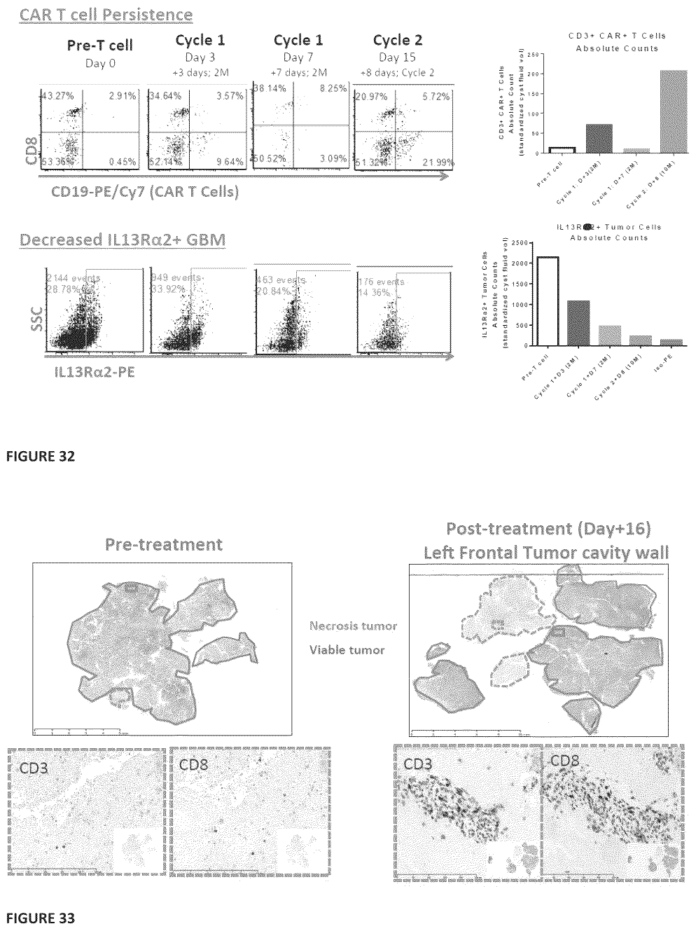

FIG. 32A presents analysis of CAR T cell persistence, as monitored by CD19. This analysis shows good T cell persistence 8 days after the Cycle 2. FIG. 32B shows decreased presence of GBM cells as monitored by IL13Rα2 expression.

FIG. 33A and FIG. 33B depict imaging results from Patient UPN097 in the region of the catheter used for intratumoral administration. In FIG. 33A one can see that few CD3+ or CD8+ T cells are present pretreatment. FIG. 33B, which is a sample at Day 16 post-treatment taken from the left frontal tumor cavity wall shows a large area of necrotic tumor and significant presence of CD3+ and CD8+ cells.

Gliomas, express IL13 receptors, and in particular, high-affinity IL13 receptors. However, unlike the IL13 receptor, glioma cells overexpress a unique IL13Rα2 chain capable of binding IL13 independently of the requirement for IL4Rβ or γc44. Like its homolog IL4, IL13 has pleotropic immunoregulatory activity outside the CNS. Both IL13 and IL4 stimulate IgE production by B lymphocytes and suppress pro-inflammatory cytokine production by macrophages. Detailed studies using autoradiography with radiolabeled IL13 have demonstrated abundant IL13 binding on nearly all malignant glioma tissues studied. This binding is highly homogeneous within tumor sections and in single cell analysis. However, molecular probe analysis specific for IL13Rα2 mRNA did not detect expression of the glioma-specific receptor by normal brain elements and autoradiography with radiolabeled IL13 also could not detect specific IL13 binding in the normal CNS. These studies suggest that the shared IL13Rα1/IL4β/γc receptor is not expressed detectably in the normal CNS. Therefore, IL13Rα2 is a very specific cell-surface target for glioma and is a suitable target for a CAR designed for treatment of a glioma.

Certain patients may be more suitable than others to receive the disclosed methods of treatment. For instance, those patients with malignancies that highly express IL-13Rα2 may particularly benefit from treatment with the disclosed CAR T-cells. Suitability of a patient can be determined by staining a resected tumor sample from a patient to determine the amount of expression of IL-13Rα2. The sample may be scored based on the number of cells exhibiting weak, moderate, or strong staining intensity. Determining the expression level of Ki67 may also be beneficial for determining the aggressiveness of the disease. Once it has been determined that a patient is well suited to receive the disclosed CAR T cells, the patient may be treated according to the disclosed methods.

Binding of IL13-based therapeutic molecules to the broadly expressed IL13Rα1/IL4β/γc receptor complex, however, has the potential of mediating undesired toxicities to normal tissues outside the CNS, and thus limits the systemic administration of these agents. An amino acid substitution in the IL13 alpha helix A at amino acid 13 of tyrosine for the native glutamic acid selectively reduces the affinity of IL13 to the IL13Rα1/IL4β/γc receptor. Binding of this mutant (termed IL13(E13Y)) to IL13Rα2, however, was increased relative to wild-type IL13. Thus, this minimally altered IL13 analog simultaneously increases IL13's specificity and affinity for glioma cells. Therefore, CAR described herein include an IL13 containing a mutation (E to Y or E to some other amino acid such as K or R or L or V) at amino acid 13 (according to the numbering of Debinski et al. 1999 Clin Cancer Res 5:3143s). IL13 having the natural sequence also may be used, however, and can be useful, particularly in situations where the modified T cells are to be locally administered, such as by injection directly into a tumor mass.

Additionally, gliomas are known to have a generally poor patient prognosis. For example, glioblastoma multiforme (GBM) is a common malignant cancer of the CNS. The 1-year and 2-year relative survival rates for GBM are 29.6% and 9.0%, respectively. Only 3.4% of patients with a GBM diagnosis survive more than 5 years. Furthermore, recurrence following surgical resection and/or treatment with other conventional therapeutics is common. Current conventional treatments include, but are not limited to, radiation therapy, small molecules (e.g. temozolomide, irinotecan, imatinib mesylate, erlotinib, and hydroxyurea), and biologics such as antibodies (e.g. bevacizumab).

The disclosed methods of treatment improve clinical prognosis in patients compared to current standards. For instance, the disclosed methods can increase 1-year, 2-year, and 5-year survival rates. In some embodiments, the 1-year survival rate of a patient being treated according to the disclosed methods can at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100%. In some embodiments, the 2-year survival rate of a patient being treated according to the disclosed methods can at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100%. In some embodiments, the 5-year survival rate of a patient being treated according to the disclosed methods can at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100%.

In some embodiments, the disclosed methods also increase the life expectancy of a patient compared to another patient receiving conventional treatments or SOC treatment, including radiation therapy, small molecule drug therapy, therapeutic biologics like therapeutic antibodies, or a combination thereof. In some embodiments, in which the patient receiving SOC treatment can expect to survive about 15 months from initial diagnosis (overall survival or OS), the patient receiving the disclosed treatment can expect an OS of 25, 26, 27, 28, 29, 30, 31, 32, 33, 34, 35, 36 months or more. In some embodiments, the patient receiving the claimed treatment can expect an OS of 42, 48, 54, 60, 66, 72, 78, 84, 90 months or more.

The disclosed methods may improve a patient's prognosis through a variety of clinical outcomes. For instance, the disclosed methods can result in a reduction in tumor volume in a patient being treated with a composition comprising T cells. In some embodiments, the disclosed methods of treatment can result in at least a 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% reduction in tumor volume. In some embodiments, the tumors in a patient may be completely eliminated and the patient can be cured of the malignancy.

Additionally, the disclosed methods are safe and well-tolerated. Patients being treated according to the disclosed methods may not experience significant side effects, and furthermore, may be able to discontinue taking auxiliary medications. For instance, in some embodiments, the disclosed methods will not result in any grade 3 or higher toxicities according to NCJ Common Toxicity Criteria (CTC). The CTC provides a quantifiable scale of 0-5, with 0 meaning no adverse event, 1 meaning mild, 2 meaning moderate, 3 meaning sever and undesirable, 4 meaning life threatening or disabling, and 5 meaning death. Thus, side effects and or toxicities may include events like mild or moderate headaches, fatigue, myalgia, and minor nervous system disorders such as olfactory aura, but high grade toxicities will be avoided.

Steroids like dexamethasone are commonly used in the clinical management of gliomas to prevent neurological side effects like brain edema. The disclosed methods of treatment can decrease the need for such auxiliary treatments. For instance, if a patient is receiving a regimen of steroids (e.g. dexamethasone) prior to treatment according to the disclosed methods, the patient may be able to reduce the dose of the steroid regimen or discontinue the steroid regimen altogether without experiencing clinically deleterious effects.

Brain metastases of breast cancer can express HER2. Certain of the CAR described herein that are useful in treatment of malignant glioma are targeted to HER2.

The CAR described herein can be produced by any means known in the art, though preferably they are produced using recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the patient, including unselected PBMC or enriched CD3 T cells or enriched CD3 or memory T cell subsets, can be transduced with a vector for CAR expression. Central memory T cells are one useful T cell subset. Central memory T cell can be isolated from peripheral blood mononuclear cells (PBMC) by selecting for CD45RO+/CD62L+cells, using, for example, the CliniMACS® device to immunomagnetically select cells expressing the desired receptors. The cells enriched for central memory T cells can be activated with anti-CD3/CD28, transduced with, for example, a SIN lentiviral vector that directs the expression of the CAR as well as a truncated human CD19 (CD19t), a non-immunogenic surface marker for both in vivo detection and potential ex vivo selection. The activated/genetically modified central memory T cells can be expanded in vitro with IL-2/IL-15 and then cryopreserved.

### Example 1: Construction and Structure of an IL13Rα2-Specific CAR

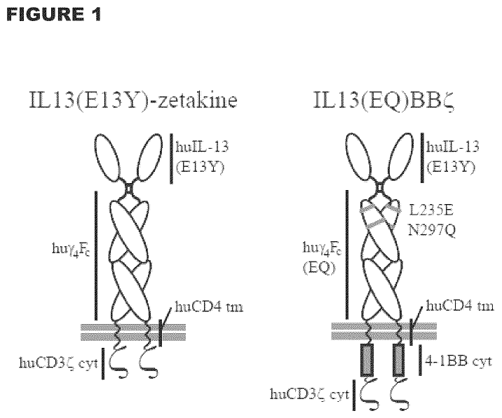

The structure of a useful IL13Rα2-specific CAR is described below. The codon optimized CAR sequence contains a membrane-tethered IL-13 ligand mutated at a single site (E13Y) to reduce potential binding to IL13Rα1, an IgG4 Fc spacer containing two mutations (L235E; N297Q) that greatly reduce Fc receptor-mediated recognition models, a CD4 transmembrane domain, a costimulatory 4-1BB cytoplasmic signaling domain, and a CD3ζ cytoplasmic signaling domain. A T2A ribosome skip sequence separates this IL13(EQ)BBζ CAR sequence from CD19t, an inert, non-immunogenic cell surface detection/selection marker. This T2A linkage results in the coordinate expression of both IL13(EQ)BBζ and CD19t from a single transcript. FIG. 1A is a schematic drawing of the 2670 nucleotide open reading frame encoding the IL13(EQ)BBZ-T2ACD19t construct. In this drawing, the IL13Rα2-specific ligand IL13(E13Y), IgG4(EQ) Fc, CD4 transmembrane, 4-1BB cytoplasmic signaling, three-glycine linker, and CD3ζ cytoplasmic signaling domains of the IL13(EQ)BBZ CAR, as well as the T2A ribosome skip and truncated CD19 sequences are all indicated. The human GM-CSF receptor alpha and CD19 signal sequences that drive surface expression of the IL13(EQ)BBZ CAR and CD19t are also indicated. Thus, the IL13(EQ)BBZ-T2ACD19t construct includes a IL13Rα2-specific, hinge-optimized, costimulatory chimeric immunoreceptor sequence (designated IL13(EQ)BBZ), a ribosome-skip T2A sequence, and a CD19t sequence.

The IL13(EQ)BBZ sequence was generated by fusion of the human GM-CSF receptor alpha leader peptide with IL13(E13Y) ligand 5 L235E/N297Q-modified IgG4 Fc hinge (where the double mutation interferes with FcR recognition), CD4 transmembrane, 4-1BB cytoplasmic signaling domain, and CD3ζ cytoplasmic signaling domain sequences. This sequence was synthesized de novo after codon optimization. The T2A sequence was obtained from digestion of a T2A-containing plasmid. The CD19t sequence was obtained from that spanning the leader peptide sequence to the transmembrane components (i.e., basepairs 1-972) of a CD19-containing plasmid. All three fragments, 1) IL13(EQ)BBZ, 2) T2A, and 3) CD19t, were cloned into the multiple cloning site of the epHIV7 lentiviral vector. When transfected into appropriate cells, the vector integrates the sequence depicted schematically in FIG. 1B into the host cells genome. FIG. 1C provides a schematic drawing of the 9515 basepair IL13(EQ)BBZ-T2A-CD19t_epHIV7 plasmid itself.

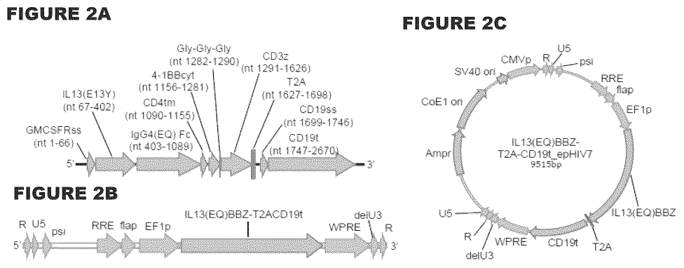

As shown schematically in FIG. 2, IL13(EQ)BBZ CAR differs in several important respects from a previously described IL13Rα2-specific CAR referred to as IL13(E13Y)-zetakine (Brown et al. 2012 Clinical Cancer Research 18:2199). The IL13(E13Y)-zetakine is composed of the IL13Rα2-specific human IL-13 mutein (huIL-13(E13Y)), human IgG4 Fc spacer (huγ4Fc), human CD4 transmembrane (huCD4 tm), and human CD3ζ chain cytoplasmic (huCD3ζ cyt) portions as indicated. In contrast, the IL13(EQ)BBζ) has two point mutations, L235E and N297Q that are located in the CH2 domain of the IgG4 spacer, and a costimulatory 4-1BB cytoplasmic domain (4-1BB cyt).

### Example 2: Construction and Structure of epHIV7 Used for Expression of an IL13Rα2-specific CAR

The pHIV7 plasmid is the parent plasmid from which the clinical vector IL13(EQ)BBZ-T2A-CD19t_epHIV7 was derived in the T cell Therapeutics Research Laboratory (TCTRL) at City of Hope (COH). The epHIV7 vector used for expression of the CAR was produced from pHIV7 vector. Importantly, this vector uses the human EF1 promoter to drive expression of the CAR. Both the 5′ and 3′ sequences of the vector were derived from pv653RSN as previously derived from the HXBc2 provirus. The polypurine tract DNA flap sequences (cPPT) were derived from HIV-1 strain pNL4-3 from the NIH AIDS Reagent Repository. The woodchuck post-transcriptional regulatory element (WPRE) sequence was previously described.

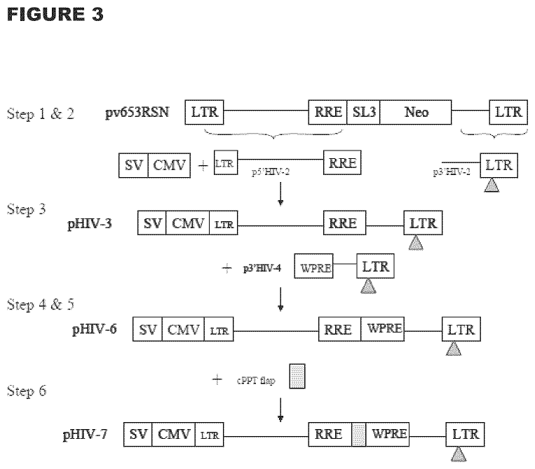

Construction of pHIV7 is schematically depicted in FIG. 3. Briefly, pv653RSN, containing 653 bp from gag-pol plus 5′ and 3′ long-terminal repeats (LTRs) with an intervening SL3-neomycin phosphotransferase gene (Neo), was subcloned into pBluescript, as follows: In Step 1, the sequences from 5′ LTR to rev-responsive element (RRE) made p5′HIV-1 51, and then the 5′ LTR was modified by removing sequences upstream of the TATA box, and ligated first to a CMV enhancer and then to the SV40 origin of replication (p5′HIV-2). In Step 2, after cloning the 3′ LTR into pBluescript to make p3′HIV-1, a 400-bp deletion in the 3′ LTR enhancer/promoter was made to remove cis-regulatory elements in HIV U3 and form p3′HIV-2. In Step 3, fragments isolated from the p5′HIV-3 and p3′HIV-2 were ligated to make pHIV-3. In Step 4, the p3′HIV-2 was further modified by removing extra upstream HIV sequences to generate p3′HIV-3 and a 600-bp BamHI-SalI fragment containing WPRE was added to p3′HIV-3 to make the p3′HIV-4. In Step 5, the pHIV-3 RRE was reduced in size by PCR and ligated to a 5′ fragment from pHIV-3 (not shown) and to the p3′HIV-4, to make pHIV-6. In Step 6, a 190-bp BglII-BamHI fragment containing the cPPT DNA flap sequence from HIV-1 pNL4-3 (55) was amplified from pNL4-3 and placed between the RRE and the WPRE sequences in pHIV6 to make pHIV-7. This parent plasmid pHIV7-GFP (GFP, green fluorescent protein) was used to package the parent vector using a four-plasmid system.

A packaging signal, psi yr, is required for efficient packaging of viral genome into the vector. The RRE and WPRE enhance the RNA transcript transport and expression of the transgene. The flap sequence, in combination with WPRE, has been demonstrated to enhance the transduction efficiency of lentiviral vector in mammalian cells.

The helper functions, required for production of the viral vector), are divided into three separate plasmids to reduce the probability of generation of replication competent lentivirus via recombination: 1) pCgp encodes the gag/pol protein required for viral vector assembly; 2) pCMV-Rev2 encodes the Rev protein, which acts on the RRE sequence to assist in the transportation of the viral genome for efficient packaging; and 3) pCMV-G encodes the glycoprotein of the vesiculo-stomatitis virus (VSV), which is required for infectivity of the viral vector.

There is minimal DNA sequence homology between the pHIV7 encoded vector genome and the helper plasmids. The regions of homology include a packaging signal region of approximately 600 nucleotides, located in the gag/pol sequence of the pCgp helper plasmid; a CMV promoter sequence in all three helper plasmids; and a RRE sequence in the helper plasmid pCgp. It is highly improbable that replication competent recombinant virus could be generated due to the homology in these regions, as it would require multiple recombination events. Additionally, any resulting recombinants would be missing the functional LTR and tat sequences required for lentiviral replication.

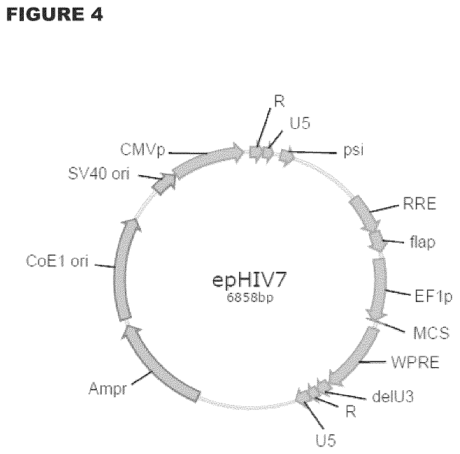

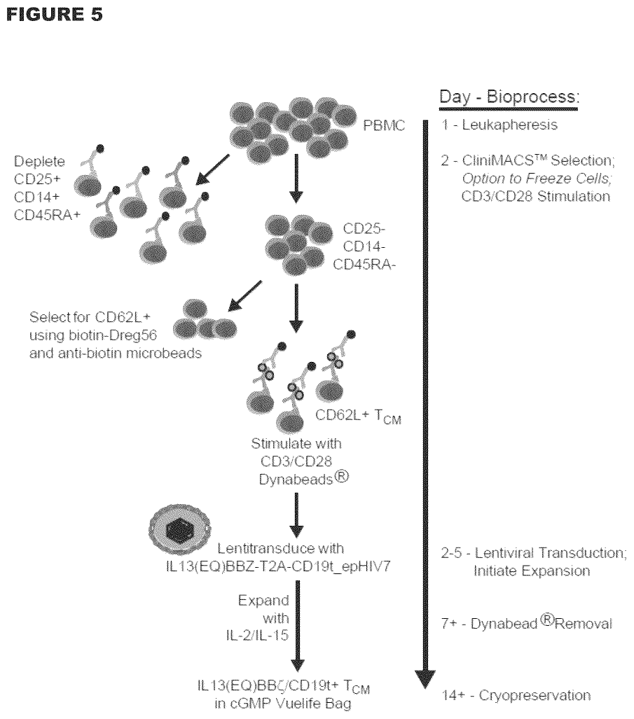

The CMV promoter was replaced by the EF1α-HTLV promoter (EF1p), and the new plasmid was named epHIV7 (FIG. 4). The EF1p has 563 bp and was introduced into epHIV7 using NruI and NheI, after the CMV promoter was excised.

The lentiviral genome, excluding gag/pol and rev that are necessary for the pathogenicity of the wild-type virus and are required for productive infection of target cells, has been removed from this system. In addition, the IL13(EQ)BBZ-T2ACD19t_epHIV7 vector construct does not contain an intact 3′LTR promoter, so the resulting expressed and reverse transcribed DNA proviral genome in targeted cells will have inactive LTRs. As a result of this design, no HIV-I derived sequences will be transcribed from the provirus and only the therapeutic sequences will be expressed from their respective promoters. The removal of the LTR promoter activity in the SIN vector is expected to significantly reduce the possibility of unintentional activation of host genes. Table 4 summarizes the various regulator elements present in IL13(EQ)BBZ-T2ACD19t_epHIV7.

TABLE 4
Functional elements of IL13(EQ)41BBZ-T2A-CD19t_epHIV7

| Regulatory Elements | Location | |
|---|---|---|
| and Genes | (Nucleotide Numbers) | Comments |
| U5 | 87-171 | 5′ Unique sequence |
| psi | 233-345 | Packaging signal |
| RRE | 957-1289 | Rev-responsive element |
| flap | 1290-1466 | Contains polypurine track |
| | | sequence and central termination |
| | | sequence to facilitate nuclear |
| | | import of pre-integration complex |
| EF1p Promoter | 1524-2067 | EF1-alpha Eukaryotic Promoter |
| | | sequence driving expression of |
| | | CD19Rop |
| IL13-IgG4 (EQ)-41BB- | 2084-4753 | Therapeutic insert |
| Zeta-T2A-CD19t | | |
| WPRE | 4790-5390 | Woodchuck hepatitis virus derived |
| | | regulatory element to enhance viral |
| | | RNA transportation |
| delU3 | 5405-5509 | 3′ U3 with deletion to generate |
| | | SIN vector |
| R | 5510-5590 | Repeat sequence within LTR |
| U5 | 5591-5704 | 3′ U5 sequence in LTR |
| Amp^{R} | 6540-7398 | Ampicillin-resistance gene |
| CoE1 ori | 7461-8342 | Replication origin of plasmid |
| SV40 ori | 8639-8838 | Replication origin of SV40 |
| CMV promoter | 8852-9451 | CMV promoter to generate |
| | | viral genome RNA |
| R | 9507-86 | Repeat sequence within LTR |

### Example 3: Production of Vectors for Transduction of Patient T Cells

For each plasmid (IL13(EQ)BBZ-T2A-CD19t_epHIV7; pCgp; pCMV-G; and pCMV-Rev2), a seed bank is generated, which is used to inoculate the fermenter to produce sufficient quantities of plasmid DNA. The plasmid DNA is tested for identity, sterility and endotoxin prior to its use in producing lentiviral vector.

Briefly, cells were expanded from the 293T working cell (WCB), which has been tested to confirm sterility and the absence of viral contamination. A vial of 293T cells from the 293T WCB was thawed. Cells were grown and expanded until sufficient numbers of cells existed to plate an appropriate number of 10 layer cell factories (CFs) for vector production and cell train maintenance. A single train of cells can be used for production.

The lentiviral vector was produced in sub-batches of up to 10 CFs. Two sub-batches can be produced in the same week leading to the production of approximately 20 L of lentiviral supernatant/week. The material produced from all sub-batches were pooled during the downstream processing phase, in order to produce one lot of product. 293T cells were plated in CFs in 293T medium (DMEM with 10% FBS). Factories were placed in a 37° C. incubator and horizontally leveled in order to get an even distribution of the cells on all the layers of the CF. Two days later, cells were transfected with the four lentiviral plasmids described above using the CaPO4 method, which involves a mixture of Tris:EDTA, 2M CaCl2, 2×HBS, and the four DNA plasmids. Day 3 after transfection, the supernatant containing secreted lentiviral vectors was collected, purified and concentrated. After the supernatant was removed from the CFs, End-of-Production Cells were collected from each CF. Cells were trypsinized from each factory and collected by centrifugation. Cells were resuspended in freezing medium and cryopreserved.

These cells were later used for replication-competent lentivirus (RCL) testing.

To purify and formulate vectors crude supernatant was clarified by membrane filtration to remove the cell debris. The host cell DNA and residual plasmid DNA were degraded by endonuclease digestion (Benzonase®). The viral supernatant was clarified of cellular debris using a 0.45 m filter. The clarified supernatant was collected into a pre-weighed container into which the Benzonase® is added (final concentration 50 U/mL). The endonuclease digestion for residual plasmid DNA and host genomic DNA as performed at 37° C. for 6 h. The initial tangential flow ultrafiltration (TFF) concentration of the endonuclease-treated supernatant was used to remove residual low molecular weight components from the crude supernatant, while concentrating the virus ˜20 fold. The clarified endonuclease-treated viral supernatant was circulated through a hollow fiber cartridge with a NMWCO of 500 kD at a flow rate designed to maintain the shear rate at ˜4,000 sec-1 or less, while maximizing the flux rate. Diafiltration of the nuclease-treated supernatant was initiated during the concentration process to sustain the cartridge performance. An 80% permeate replacement rate was established, using 4% lactose in PBS as the diafiltration buffer. The viral supernatant was brought to the target volume, representing a 20-fold concentration of the crude supernatant, and the diafiltration was continued for 4 additional exchange volumes, with the permeate replacement rate at 100%.

Further concentration of the viral product was accomplished by using a high speed centrifugation technique. Each sub-batch of the lentivirus was pelleted using a Sorvall RC-26 plus centrifuge at 6000 RPM (6,088 RCF) at 6° C. for 16-20 h. The viral pellet from each sub-batch was then reconstituted in a 50 mL volume with 4% lactose in PBS. The reconstituted pellet in this buffer represents the final formulation for the virus preparation. The entire vector concentration process resulted in a 200-fold volume reduction, approximately. Following the completion of all of the sub-batches, the material was then placed at −80° C., while samples from each sub-batch were tested for sterility. Following confirmation of sample sterility, the sub-batches were rapidly thawed at 37° C. with frequent agitation. The material was then pooled and manually aliquoted in the Class II Type A/B3 biosafety cabinet in the viral vector suite. A fill configuration of 1 mL of the concentrated lentivirus in sterile USP class 6, externally threaded O-ring cryovials was used. Center for Applied Technology Development (CATD)'s Quality Systems (QS) at COH released all materials according to the Policies and Standard Operating Procedures for the CBG and in compliance with current Good Manufacturing Practices (cGMPs).

To ensure the purity of the lentiviral vector preparation, it was tested for residual host DNA contaminants, and the transfer of residual host and plasmid DNA. Among other tests, vector identity was evaluated by RT-PCR to ensure that the correct vector is present. All release criteria were met for the vector intended for use in this study.

### Example 4: Preparation of T Cells Suitable for Use in ACT

T lymphocytes are obtained from a patient by leukopheresis, and the appropriate allogenic or autologous T cell subset, for example, Central Memory T cells (T_{CM}), are genetically altered to express the CAR, then administered back to the patient by any clinically acceptable means, to achieve anti-cancer therapy.

Suitable T_{CM} can be prepared as follows. Apheresis products obtained from consented research participants are ficolled, washed and incubated overnight. Cells are then depleted of monocyte, regulatory T cell and naïve T cell populations using GMP grade anti-CD14, anti-CD25 and anti-CD45RA reagents (Miltenyi Biotec) and the CliniMACS™ separation device. Following depletion, negative fraction cells are enriched for CD62L+ T_{CM} cells using DREG56-biotin (COH clinical grade) and anti-biotin microbeads (Miltenyi Biotec) on the CliniMACS™ separation device.

Following enrichment, T_{CM} cells are formulated in complete X-Vivo15 plus 50 IU/mL IL-2 and 0.5 ng/mL IL-15 and transferred to a Teflon cell culture bag, where they are stimulated with Dynal ClinEx™ Vivo CD3/CD28 beads. Up to five days after stimulation, cells are transduced with IL13(EQ)BBZ-T2A-CD19t_epHIV7 lentiviral vector at a multiplicity of infection (MOI) of 1.0 to 0.3. Cultures are maintained for up to 42 days with addition of complete X-Vivo15 and IL-2 and IL-15 cytokine as required for cell expansion (keeping cell density between 3×10⁵ and 2×10⁶ viable cells/mL, and cytokine supplementation every Monday, Wednesday and Friday of culture). Cells typically expand to approximately 10⁹ cells under these conditions within 21 days. At the end of the culture period cells are harvested, washed twice and formulated in clinical grade cryopreservation medium (Cryostore CS5, BioLife Solutions).

On the day(s) of T cell infusion, the cryopreserved and released product is thawed, washed and formulated for re-infusion. The cryopreserved vials containing the released cell product are removed from liquid nitrogen storage, thawed, cooled and washed with a PBS/2% human serum albumin (HSA) Wash Buffer. After centrifugation, the supernatant is removed and the cells resuspended in a Preservative-Free Normal Saline (PFNS)/2% HSA infusion diluent. Samples are removed for quality control testing.

Two qualification runs on cells procured from healthy donors were performed using the manufacturing platform described above. Each preclinical qualification run product was assigned a human donor (HD) number—HD006.5 and HD187.1. Importantly, as shown in Table 5, these qualification runs expanded >80 fold within 28 days and the expanded cells expressed the IL13(EQ)BBγ/CD19t transgenes.

TABLE 5
Summary of Expression Data from Pre-clinical Qualification Run Product

| Cell | | | | | |
|---|---|---|---|---|---|
| Product | CAR | CD19 | CD4+ | CD8+ | Fold Expansion |
| HD006.5 | 20% | 22% | 24% | 76% | 84-fold (28 days) |
| Hd187.1 | 18% | 25% | 37% | 63% | 259-fold (28 days) |

### Example 5: Flow Cytometric Analysis of Surface Transgene and T Cell Marker Expression in IL13(EQ)BBγ/CD19t+ T_{CM}

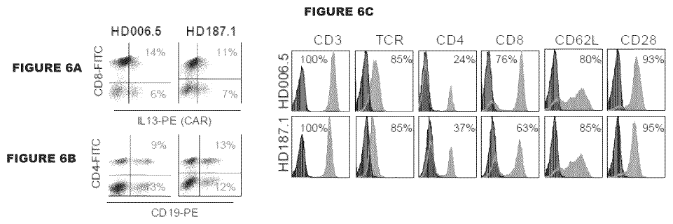

The two preclinical qualification run products described in Example 4 were used in pre-clinical studies to as described below. FIGS. 6A-C depict the results of flow cytometric analysis of surface transgene and T cell marker expression. IL13(EQ)BBγ/CD19t+ T_{CM} HD006.5 and HD187.1 were co-stained with anti-IL13-PE and anti-CD8-FITC to detect CD8+ CAR+ and CD4+ (i.e., CD8 negative) CAR+ cells (FIG. 6A), or anti-CD19-PE and anti-CD4-FITC to detect CD4+ CD19t+ and CD8+ (i.e., CD4 negative) CAR+ cells (FIG. 6B). IL13(EQ)BBγ/CD19t+ T_{CM} HD006.5 and HD187.1 were stained with fluorochrome-conjugated anti-CD3, TCR, CD4, CD8, CD62L and CD28 (grey histograms) or isotype controls (black histograms). (FIG. 6C). In each of FIGS. 6A-C, the percentages indicated are based on viable lymphocytes (DAPI negative) stained above isotype.

### Example 6: Comparison of CAR T Cell Delivery Route for Treatment of Large TS-Initiated PBT Tumors

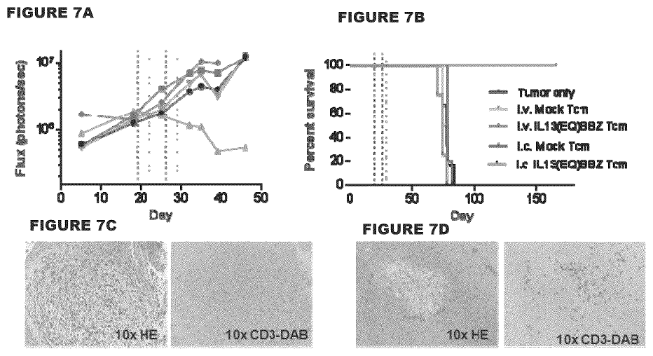

Described below are studies that compare the route of delivery, intravenous (i.v.) or intracranial (i.e.), on antitumor activity against invasive primary PBT lines. In pilot studies (data not shown), it was unexpectedly observed that i.v. administered IL13(EQ)BBζ+ TCM provided no therapeutic benefit as compared to PBS for the treatment of small (day 5) PBT030-2 EGFP:ffLuc tumors. This is in contrast to the robust therapeutic efficacy observed with i.e. administered CAR+ T cells. Reasoning that day 5 PBT030-2 tumors may have been too small to recruit therapeutic T cells from the periphery, a comparison was made of i.v. versus i.e. delivery against larger day 19 PBT030-2 EGFP:ffLuc tumors. For these studies, PBT030-2 engrafted mice were treated with either two i.v. infusions (5×10⁶ CAR+ TCM; days 19 and 26) or four i.e. infusions (1×10⁶ CAR+ TCM; days 19, 22, 26 and 29) of IL13(EQ)BBZ+ TCM, or mock TCM (no CAR). Here too no therapeutic benefit as monitored by Xenogen imaging or Kaplan-Meier survival analysis for i.v. administered CAR+ T cells (FIGS. 7A and 7B). In contrast, potent antitumor activity was observed for i.e. administered IL13(EQ)BBζ+ TCM (FIGS. 7A-B). Next, brains from a cohort of mice 7 days post T cell injection were harvested and evaluated for CD3+ human T cells by IHC. Surprisingly, for mice treated i.v. with either mock TCM or IL13(EQ)BBζ TCM there were no detectable CD3+ human T cells in the tumor or in others mouse brain regions where human T cells typically reside (i.e. the leptomeninges) (FIG. 7C), suggesting a deficit in tumor tropism. This is in contrast to the significant number of T cells detected in the i.e. treated mice (FIG. 7D).

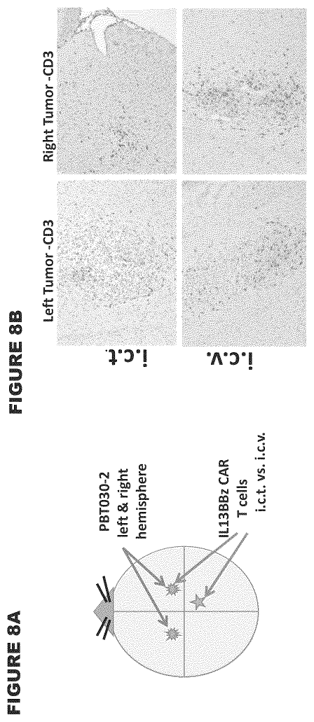

Tumor derived cytokines, particularly MCP-1/CCL2, are important in recruiting T cells to the tumor. Thus, PBT030-2 tumor cells were evaluated and it was found that this line produces high levels of MCP-1/CCL2 comparable to U251T cells (data not shown), a glioma line previously shown to attract i.v. administered effector CD8+ T cells to i.e. engrafted tumors. Malignant gliomas are highly invasive tumors and are often multi-focal in presentation. The studies described above establish that IL13BBZ T_{CM} can eliminate infiltrated tumors such as PBT030-2, and mediate long-term durable antitumor activity. The capacity of intracranially delivered CAR T cells to traffic to multifocal disease was also examined. For this study PBT030-2 EGFP:ffLuc TSs were implanted in both the left and right hemispheres (FIG. 8A) and CAR+ T cells were injected only at the right tumor site. Encouragingly, for all mice evaluated (n=3) we detected T cells by CD3 IHC 7-days post T cell infusion both at the site of injection (i.e. right tumor), as well within the tumor on the left hemisphere (FIG. 8B). These findings provide evidence that CAR+ T cells are able to traffic to and infiltrate tumor foci at distant sites. Similar findings were also observed in a second tumor model using the U251T glioma cell line (data not shown).

### Example 7: Amino acid Sequence of IL13(EQ)BBζ/CD19t

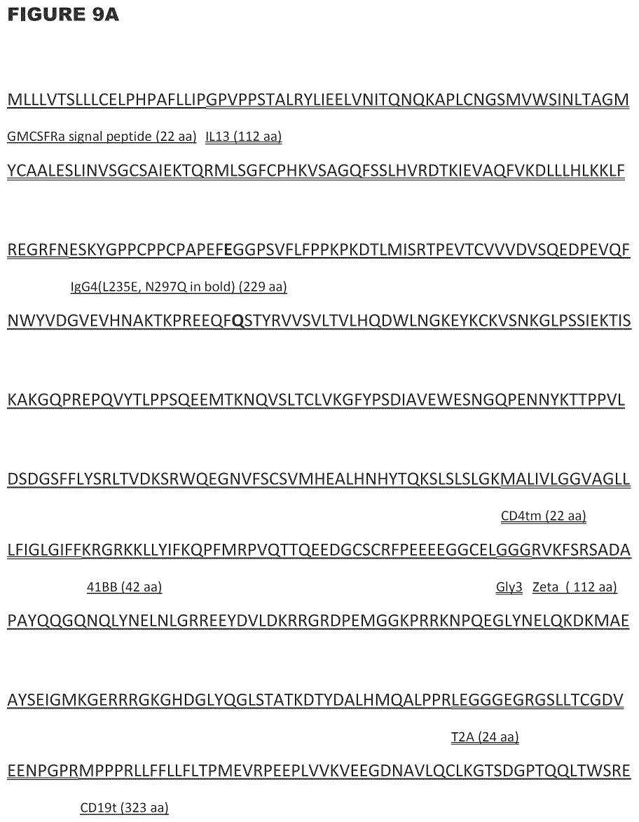

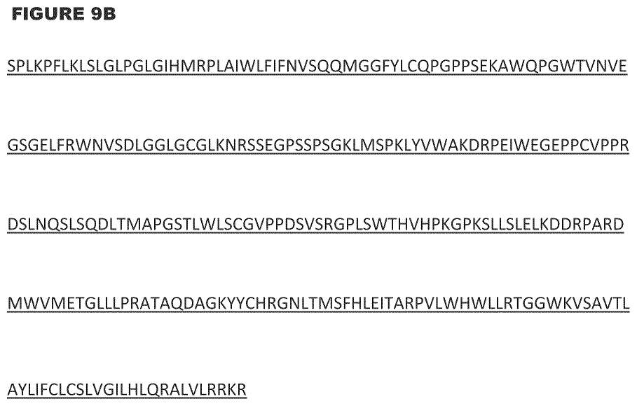

The complete amino acid sequence of IL13(EQ)BBζ/CD19t is depicted in FIGS. 9A-9B. The entire sequence (SEQ ID NO:1) includes: a 22 amino acid GMCSF signal peptide (SEQ ID NO:2), a 112 amino acid IL-13 sequence (SEQ ID NO:3; amino acid substitution E13Y shown in bold); a 229 amino acid IgG4 sequence (SEQ ID NO:4; with amino acid substitutions L235E and N297Q shown in bold); a 22 amino acid CD4 transmembrane sequence (SEQ ID NO:5); a 42 amino acid 4-1BB sequence (SEQ ID NO:6); a 3 amino acid Gly linker; a 112 amino acid CD3ζ sequence (SEQ ID NO:7); a 24 amino acid T2A sequence (SEQ ID NO:8); and a 323 amino acid CD19t sequence (SEQ ID NO:9).

The mature chimeric antigen receptor sequence (SEQ ID NO:10) includes: a 112 amino acid IL-13 sequence (SEQ ID NO:3; amino acid substitution E13Y shown in bold); a 229 amino acid IgG4 sequence (SEQ ID NO:4; with amino acid substitutions L235E and N297Q shown in bold); at 22 amino acid CD4 sequence (SEQ ID NO:5); a 42 amino acid 4-1BB sequence (SEQ ID NO:6); a 3 amino acid Gly linker; and a 112 amino acid CD3ζ sequence (SEQ ID NO:7). Within this CAR sequence (SEQ ID NO:10) is the IL-13/IgG4/CD4t/41-BB sequence (SEQ ID NO:11), which includes: a 112 amino acid IL-13 sequence (SEQ ID NO:3; amino acid substitution E13Y shown in bold); a 229 amino acid IgG4 sequence (SEQ ID NO:4; with amino acid substitutions L235E and N297Q shown in bold); at 22 amino acid CD4 sequence (SEQ ID NO:5); and a 42 amino acid 4-1BB sequence (SEQ ID NO:6). The IL13/IgG4/CD4t/4-1BB sequence (SEQ ID NO:11) can be joined to the 112 amino acid CD3ζ sequence (SEQ ID NO:7) by a linker such as a Gly Gly Gly linker. The CAR sequence (SEQ ID NO:10) can be preceded by a 22 amino acid GMCSF signal peptide (SEQ ID NO:2).

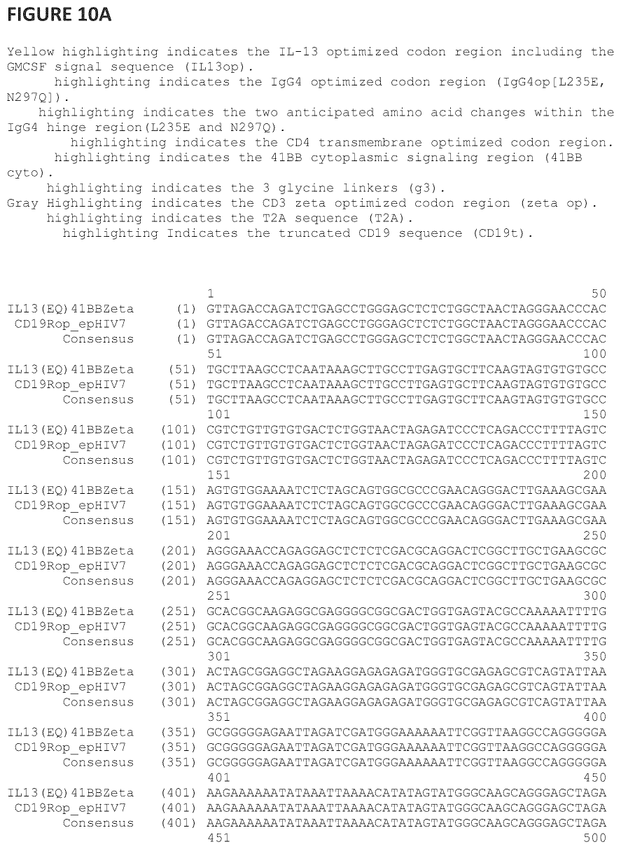

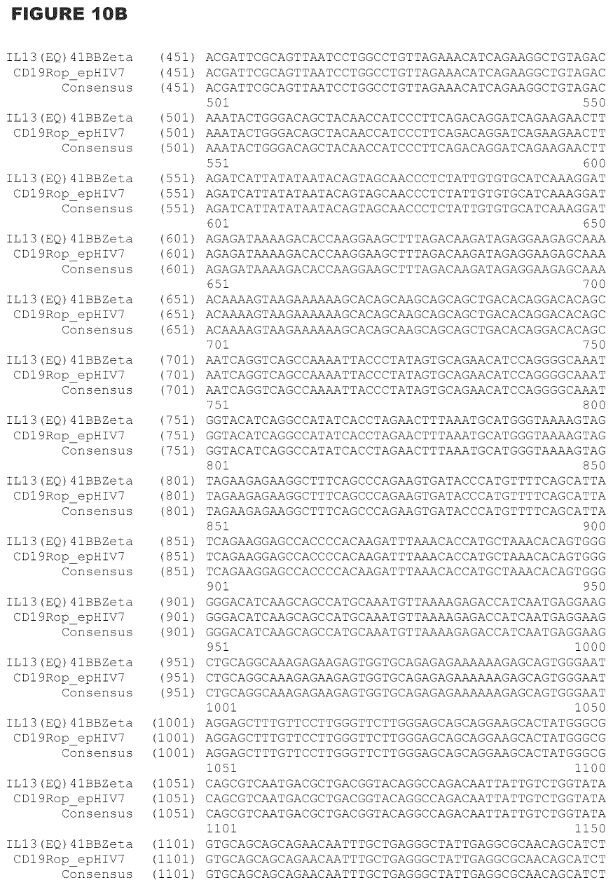

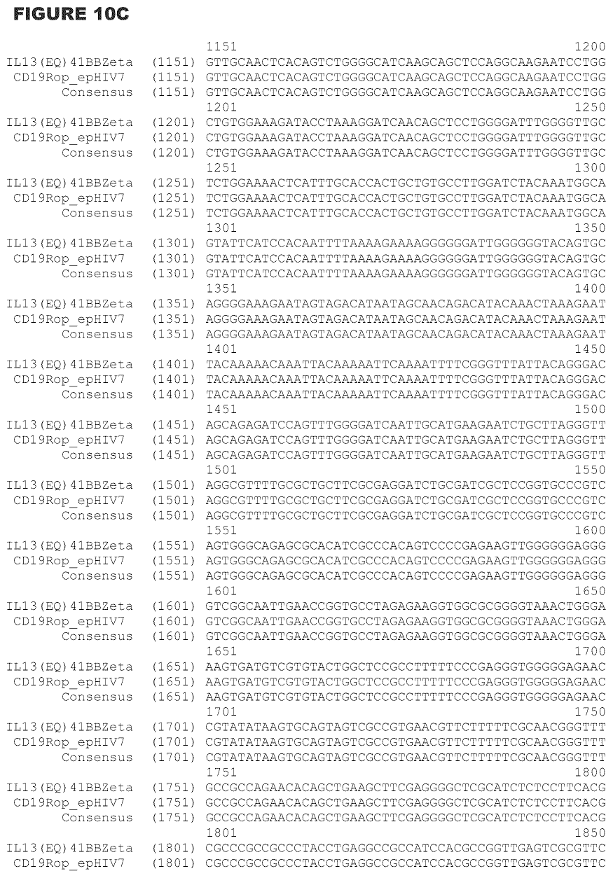

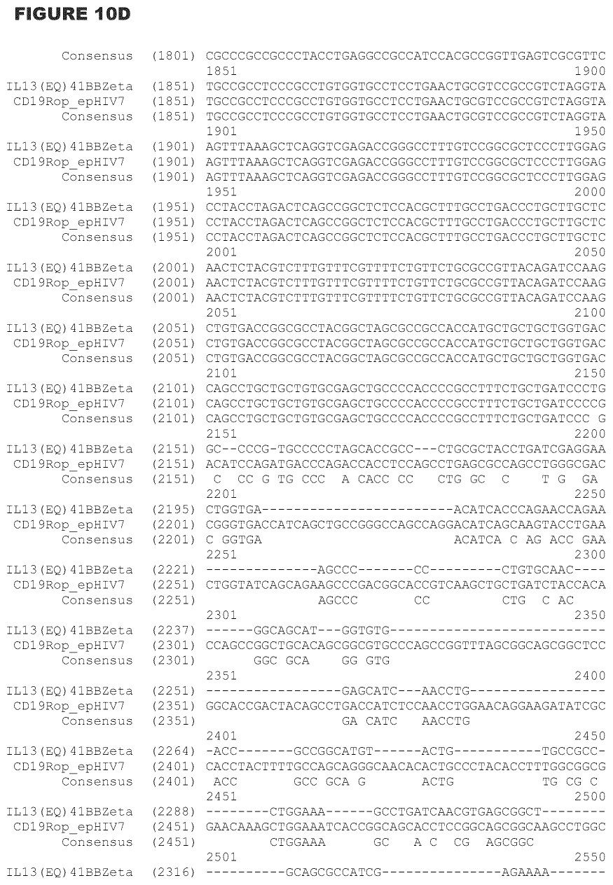

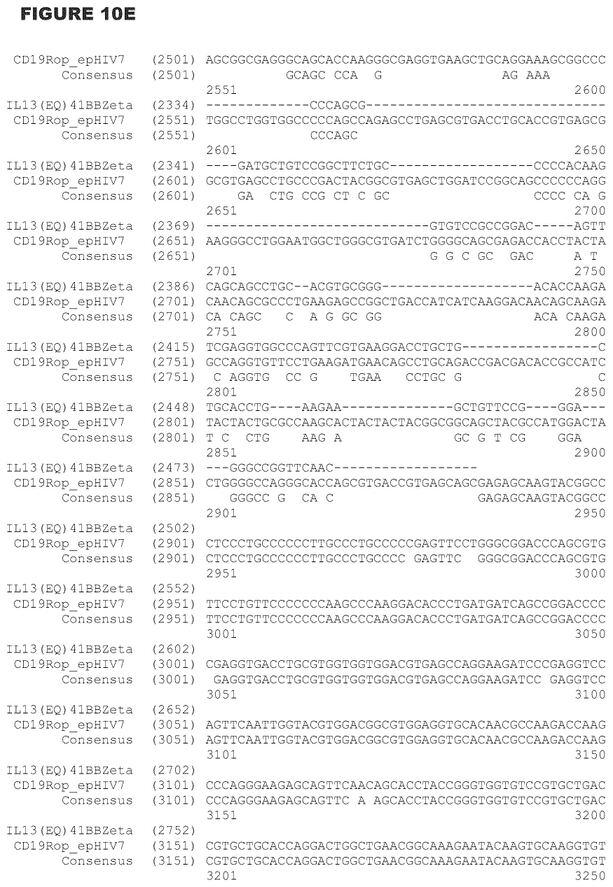

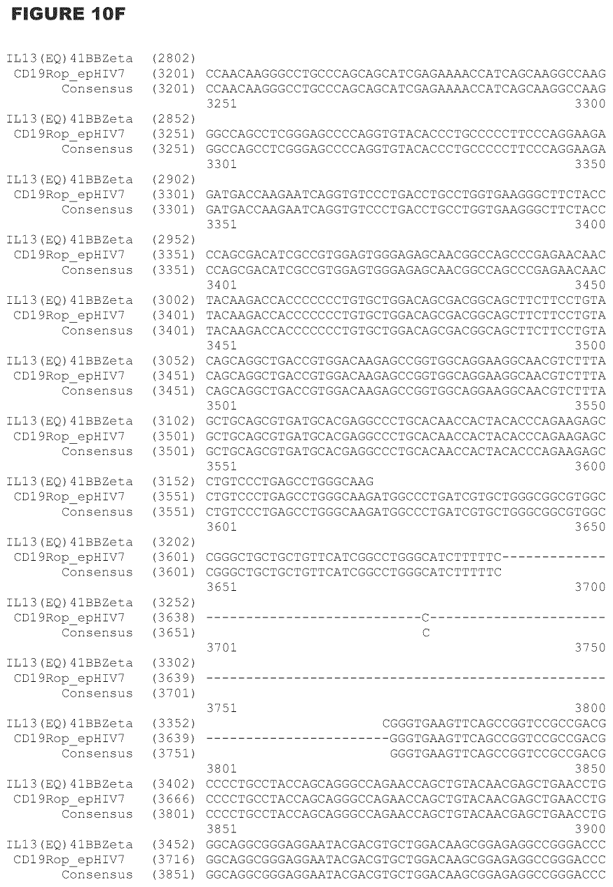

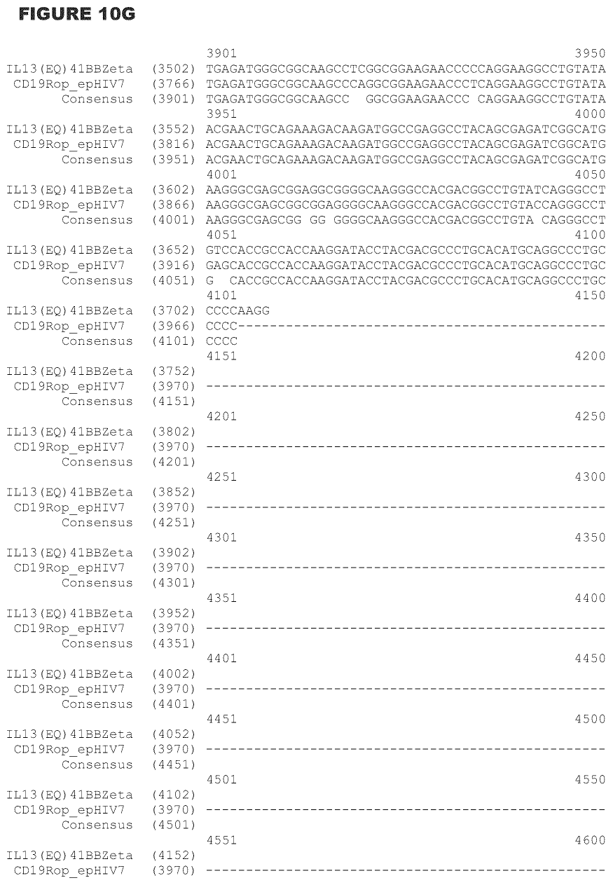

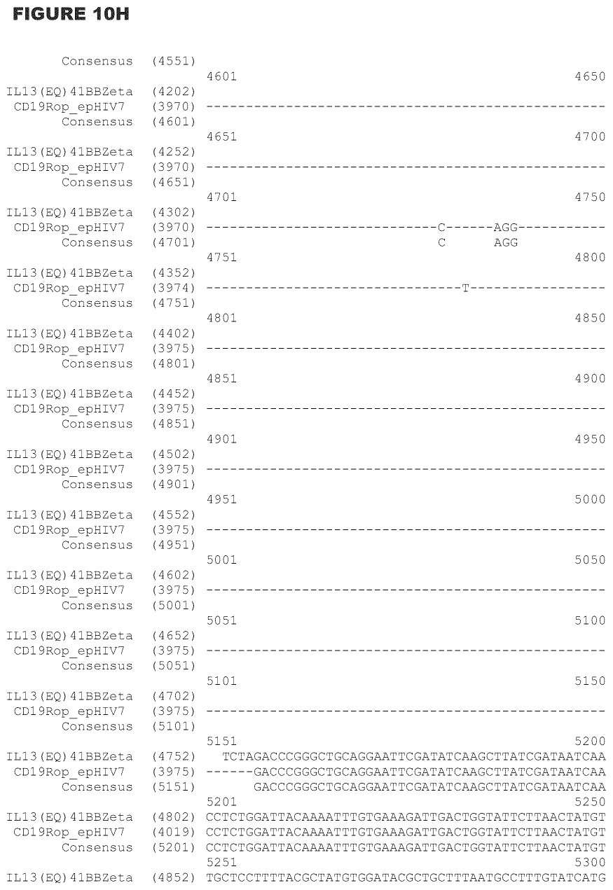

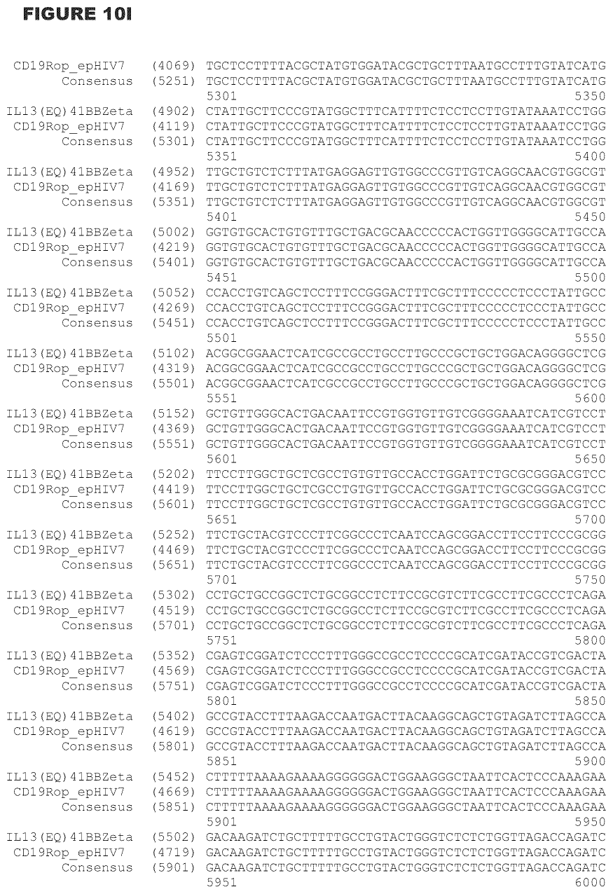

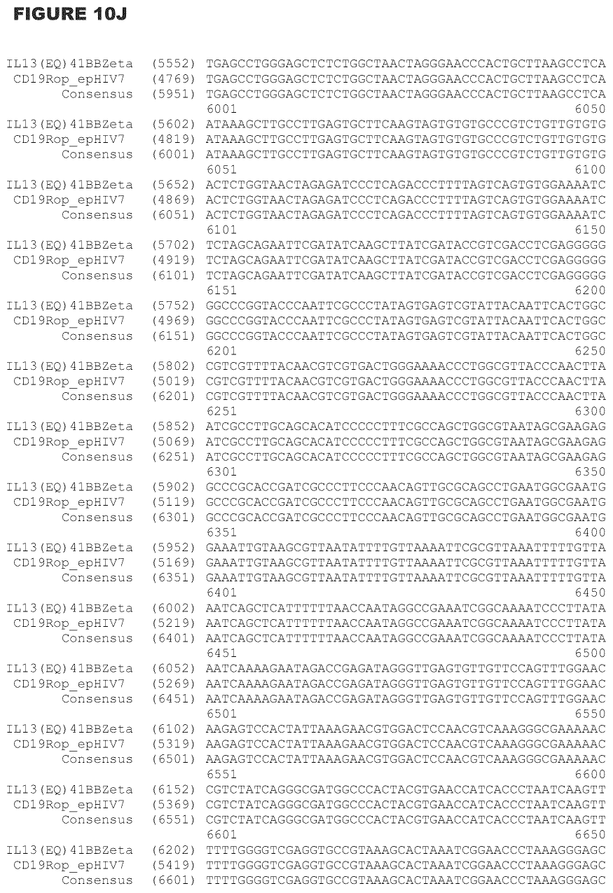

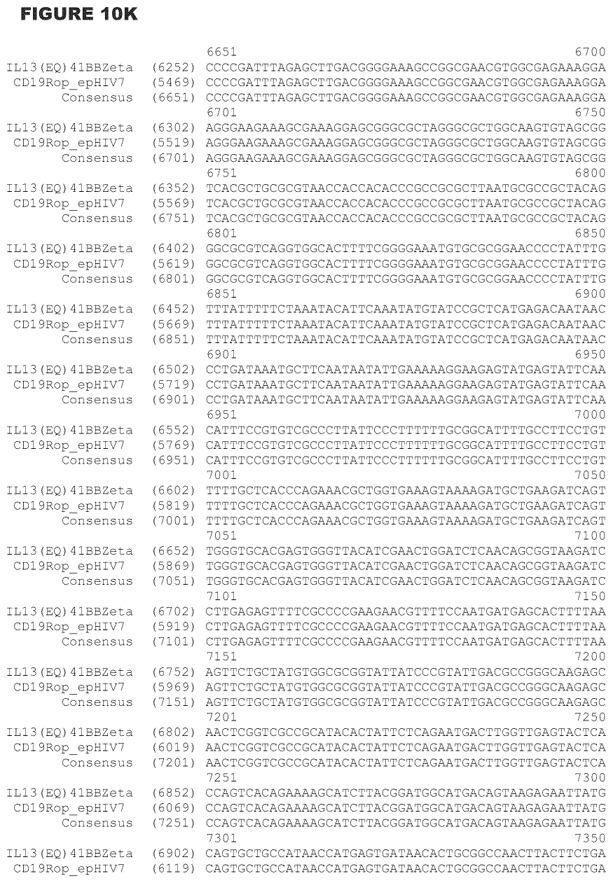

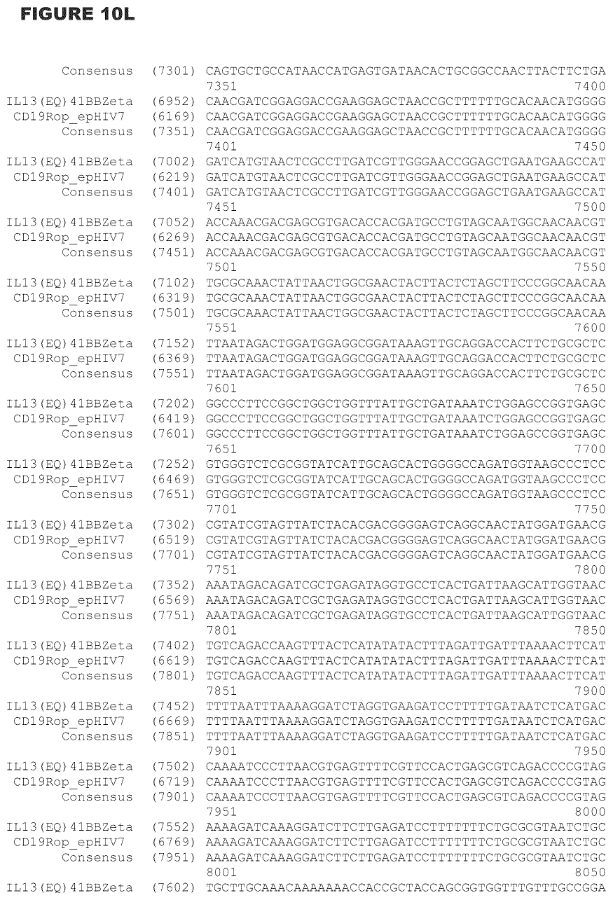

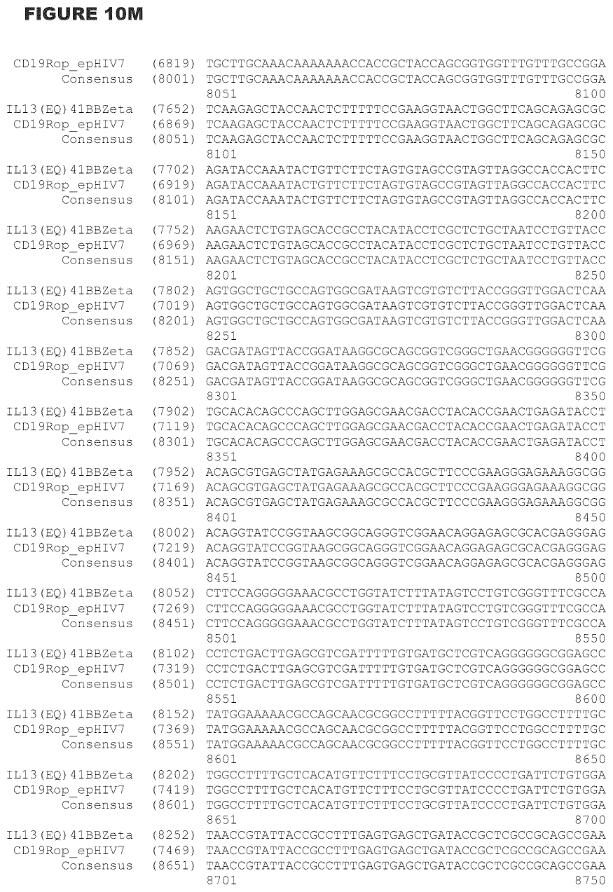

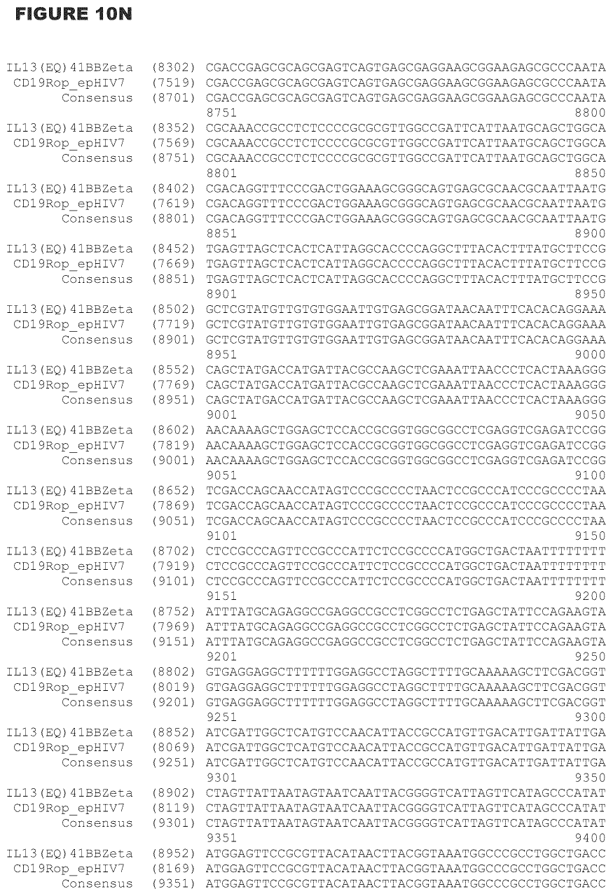

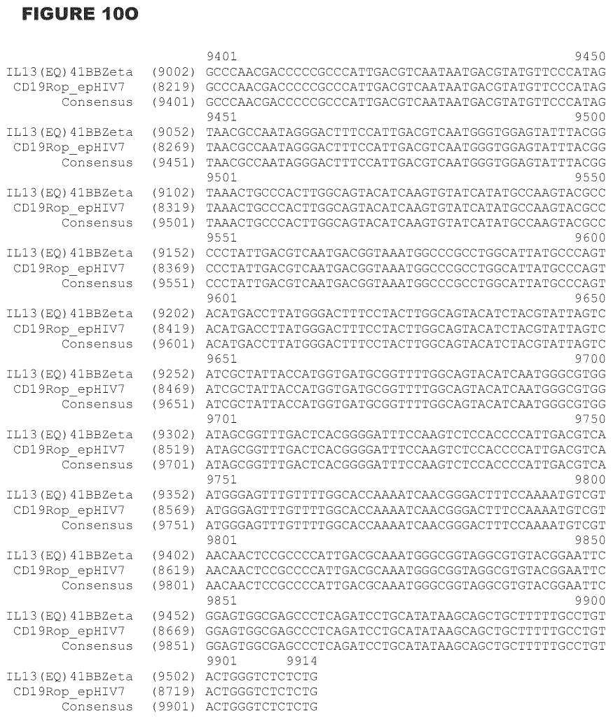

FIGS. 10A-10O depict a comparison of the sequences of IL13(EQ)41BBζ[IL13{EQ}41BBζ T2A-CD19t_epHIV7; pF02630](SEQ ID NO:12) and CD19Rop_epHIV7 (pJ01683) (SEQ ID NO:13).

### Example 8: Amino acid Sequence of Additional CAR Targeting IL13Rα2

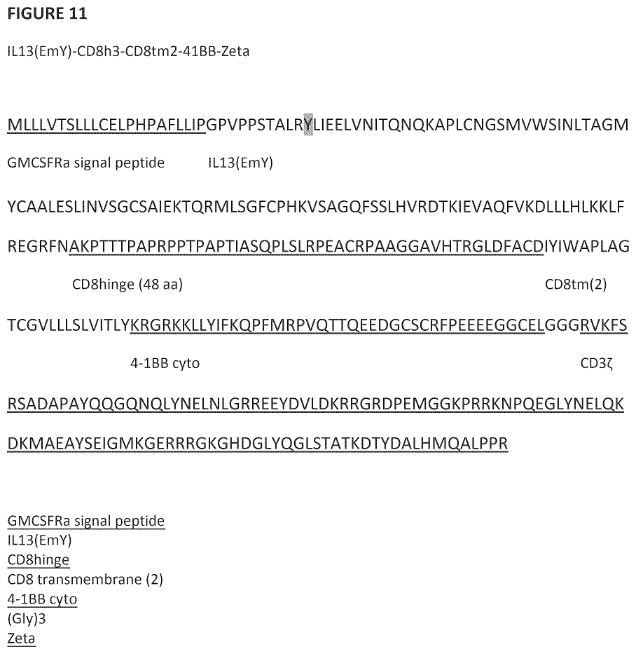

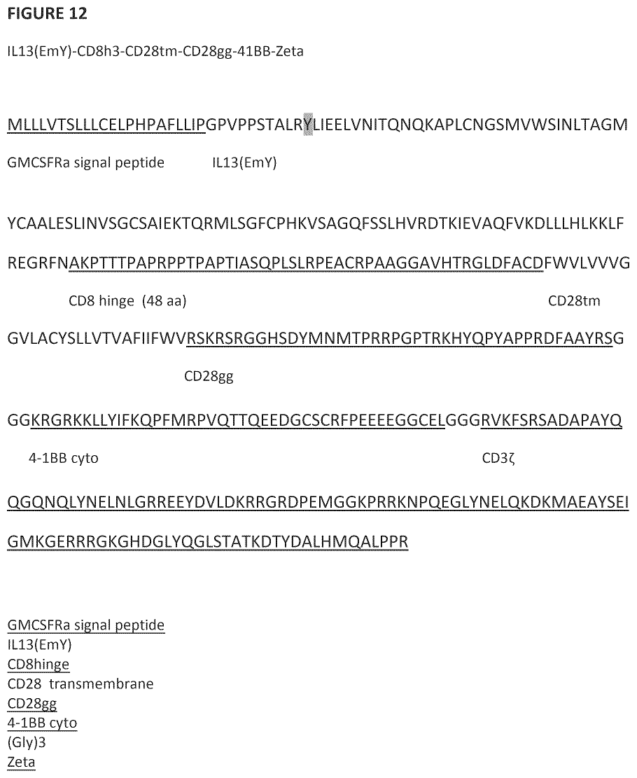

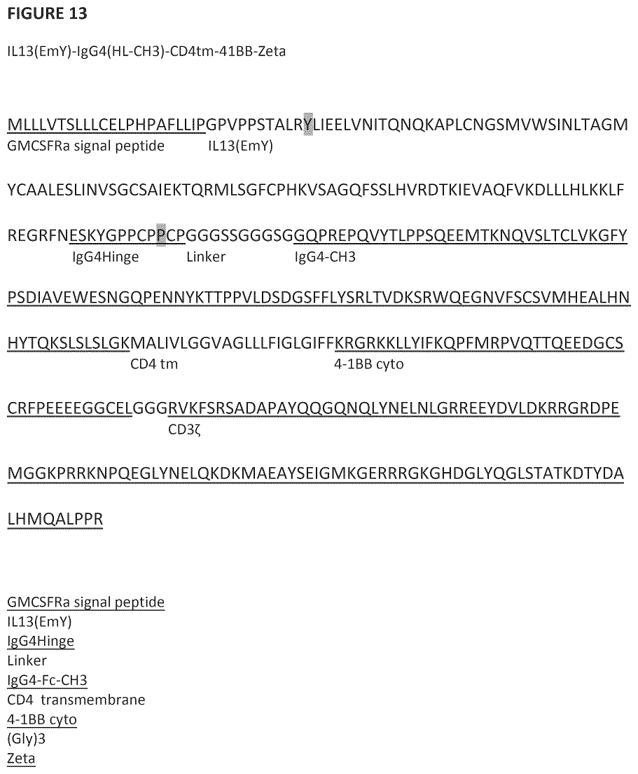

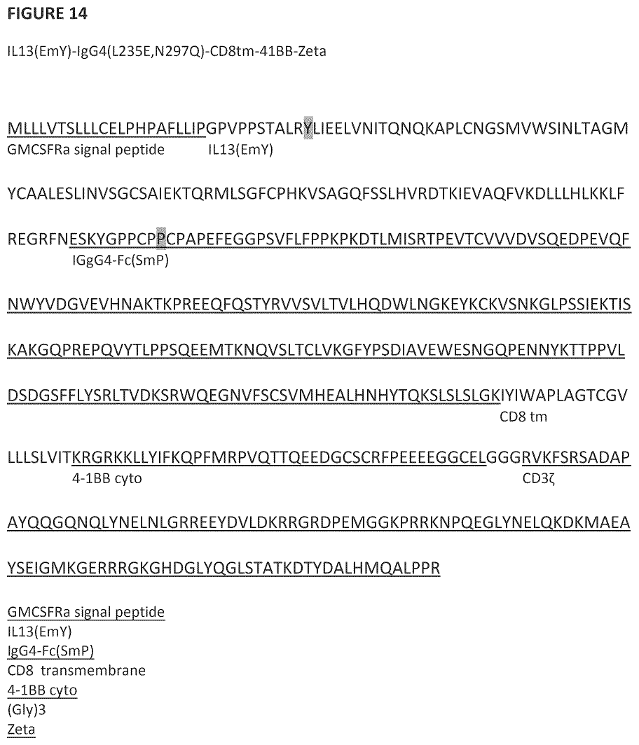

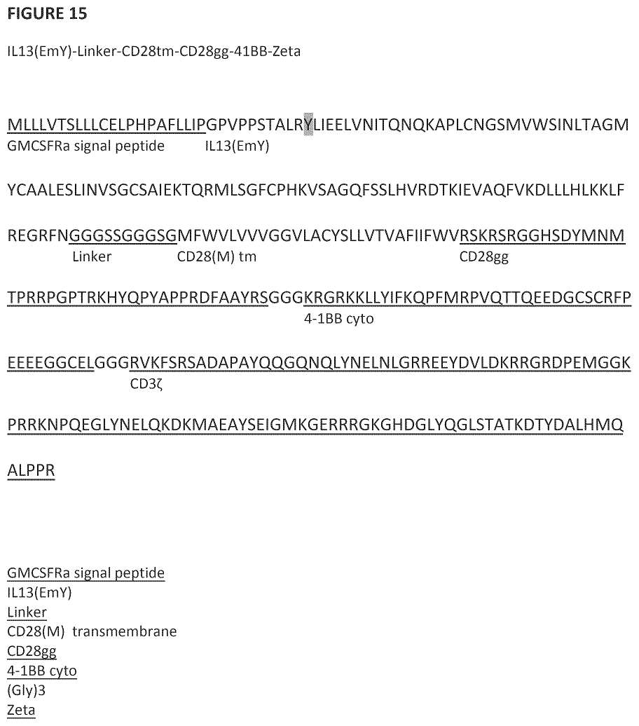

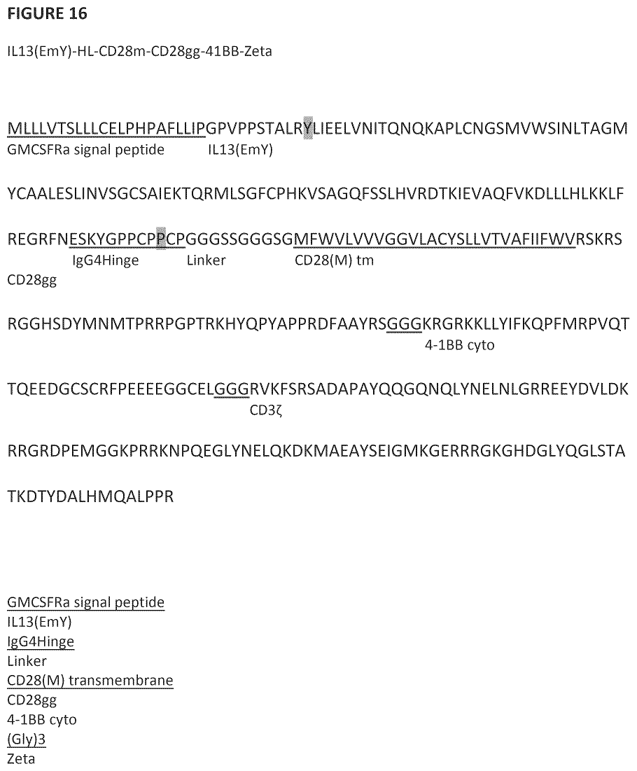

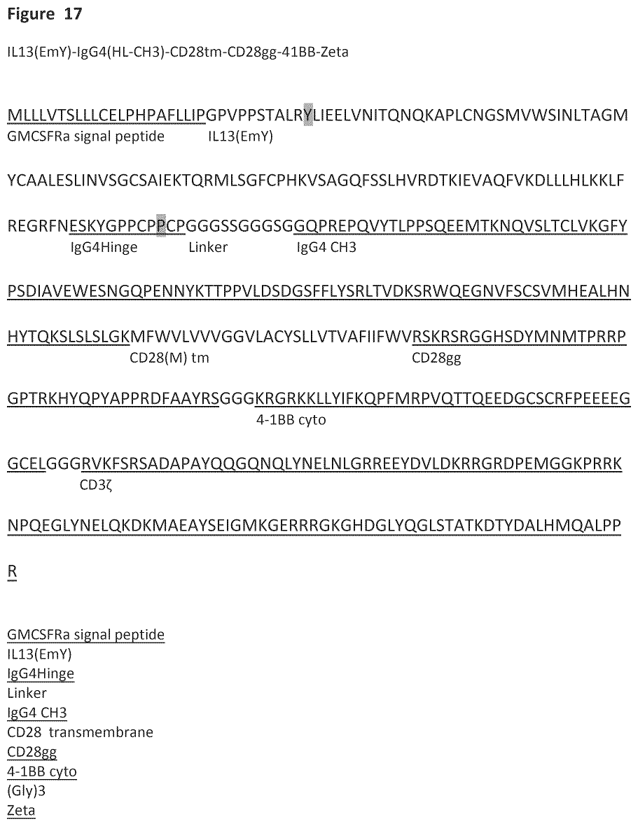

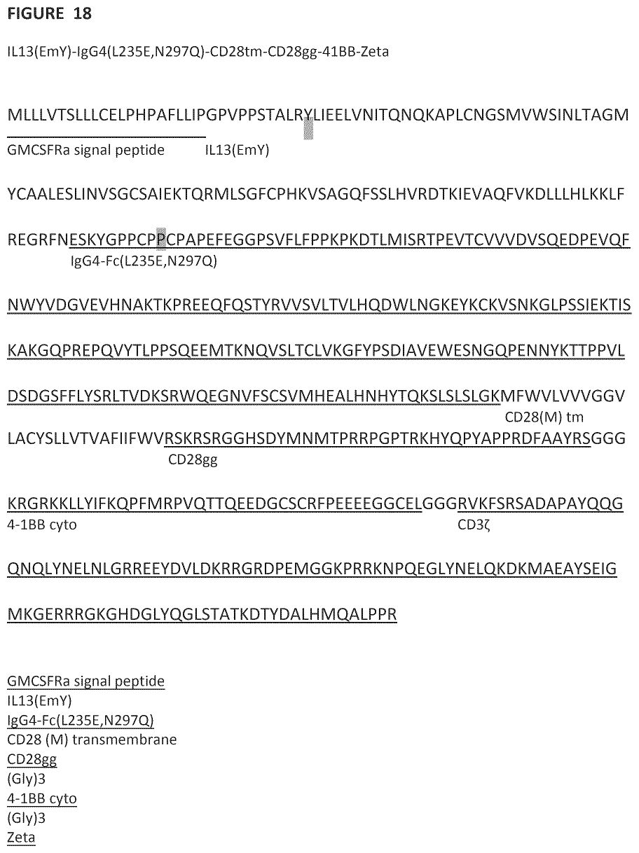

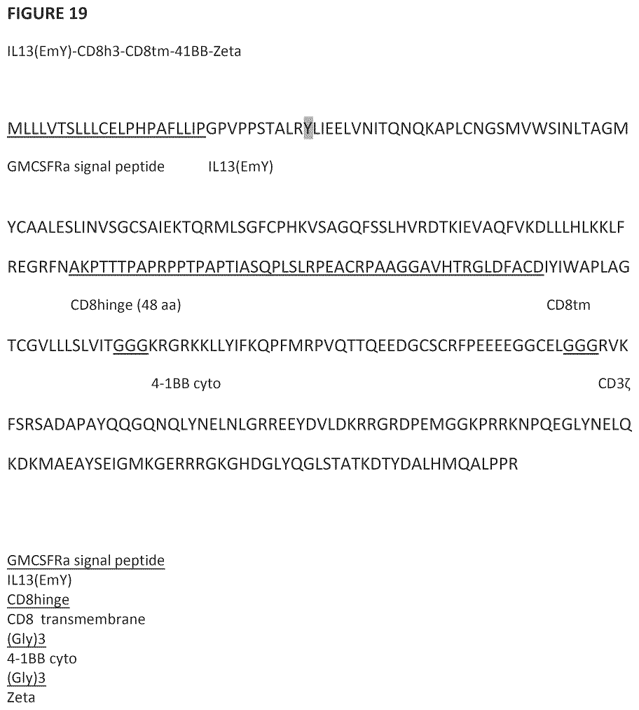

FIGS. 11-18 depict the amino acid sequences of additional CAR directed against IL13Rα2. In each case the various domains are labelled except for the GlyGlyGly spacer located between certain intracellular domains. Each includes human IL13 with and Glu to Tyr (SEQ ID NO:3; amino acid substitution E13Y shown in highlighted). In the expression vector used to express these CAR, the amino acid sequence expressed can include a 24 amino acid T2A sequence (SEQ ID NO:8); and a 323 amino acid CD19t sequence (SEQ ID NO:9) to permit coordinated expression of a truncated CD19 sequence on the surface of CAR-expressing cells.

A panel of CAR comprising human IL13(E13Y) domain, a CD28 tm domain, a CD28gg costimulatory domain, a 4-1BB costimulatory domain, and a CD3ζ domain CAR backbone and including either a HL (22 amino acids) spacer, a CD8 hinge (48 amino acids) spacer, IgG4-HL-CH3 (129 amino acids) spacer or a IgG4(EQ) (229 amino acids) spacer were tested for their ability to mediate IL13Rα2-specific killing as evaluated in a 72-hour co-culture assay. With the exception of HL (22 amino acids) which appeared to have poor CAR expression in this system, all were active.

### Example 9: Structure of Two HER2-CAR

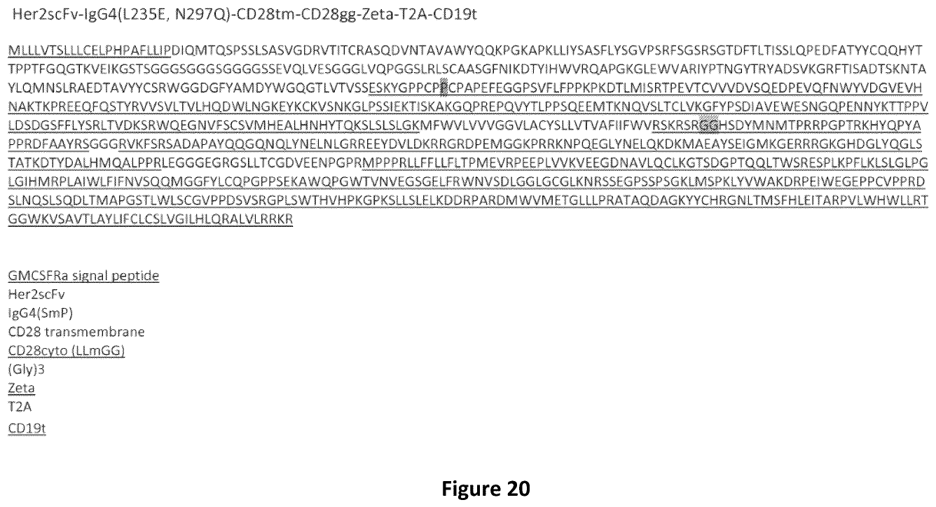

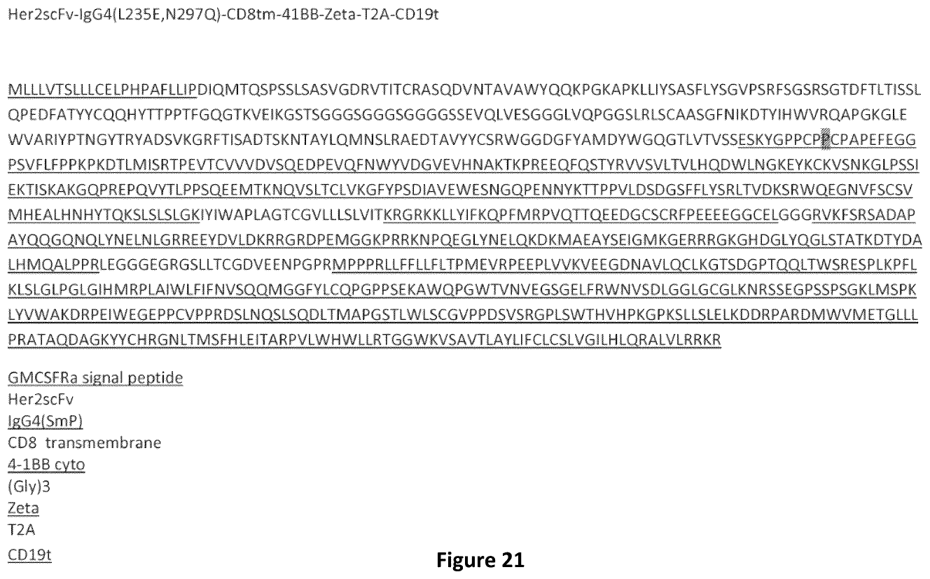

One CAR comprising a HER2 scFv described herein is referred to as Her2scFv-IgG4(L235E, N297Q)-CD28tm-CD28gg-Zeta-T2A-CD19t. This CAR includes a variety of important features including: a scFv targeted to HER2; an IgG4 Fc region that is mutated at two sites within the CH2 region (L235E; N297Q) in a manner that reduces binding by Fc receptors (FcRs); a CD28 transmembrane domain, a CD28 co-stimulatory domain, and CD3ζ activation domain. FIG. 20 presents the amino acid sequence of this CAR, including the sequence of the truncated CD19 sequence used for monitoring CAR expression and the T2A ribosomal skip sequence that allows the CAR to be produced without fusion of the truncated CD19 sequence. As shown in FIG. 21, the immature CAR includes: GMCSFR signal peptide, HER2 scFv, IgG4 that acts as a spacer, a CD8 transmembrane domain, a 4-IBB co-stimulatory domain that includes a LL to GG sequence alteration, a three Gly sequence, CD3 Zeta stimulatory domain. The transcript also encodes a T2A ribosomal sequence and a truncated CD19 sequence that are not part of the CAR protein sequence. The mature CAR is identical to the immature CAR, but lacks the GMCSF signal peptide.

### Example 10: Expression of CAR Targeted to HER2

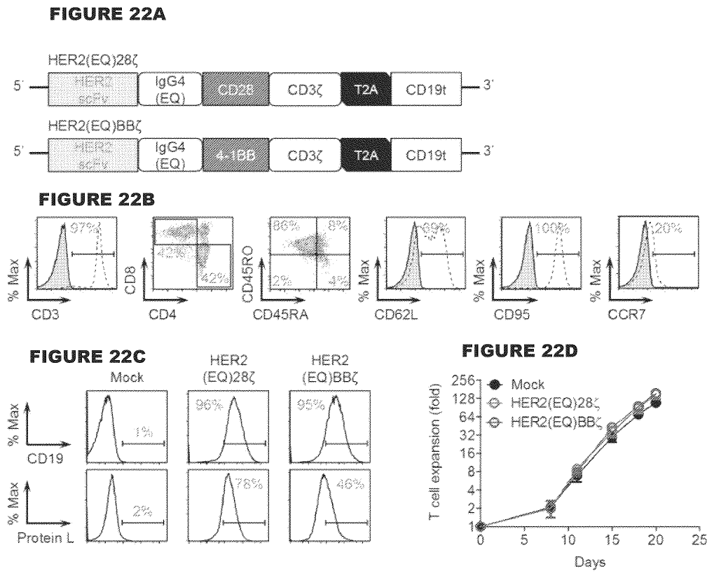

FIG. 22A is a schematic diagram of two the HER2-specific CAR constructs depicted in FIG. 20 and FIG. 21. In HER2(EQ)28ζ the scFv is tethered to the membrane by a modified IgG4 Fc linker (double mutant, L235E; N297Q), containing a CD28 transmembrane domain, an intracellular CD28 co-stimulatory domain and a cytolytic CD3ζ domain. The T2A skip sequence separates the CAR from a truncated CD19 (CD19t) protein employed for cell tracking. HER2(EQ)BBζ is similar except that the costimulatory domain is 4-1BB rather than CD28 and the transmembrane domain is a CD8 transmembrane domain rather than a CD28 transmembrane domain. Human central memory (TCM) cells were transfected with a lentiviral vector expressing either HER2(EQ)28ζ or HER2(EQ)BBζ. FIG. 22B depicts representative FACS data of human TCM surface phenotype. FIG. 22C depicts the results of assays for CD19 and Protein L expression in TCM transfected with a lentiviral vector expressing either HER2(EQ)28ζ or HER2(EQ)BBζ. As can be seen from these results, transfection efficiency as assessed by CD19 expression was similar for both CAR. However, Protein L expression was lower for HER2(EQ)BBC than for HER2(EQ)28ζ suggesting that the HER2(EQ)BBζ CAR is less stable that the HER2(EQ)BBζ. Analysis of cell expansion (FIG. 22D) shows that neither CAR interferes with T cell expansion.

### Example 11: In Vitro Characterization of HER2 Targeted CAR

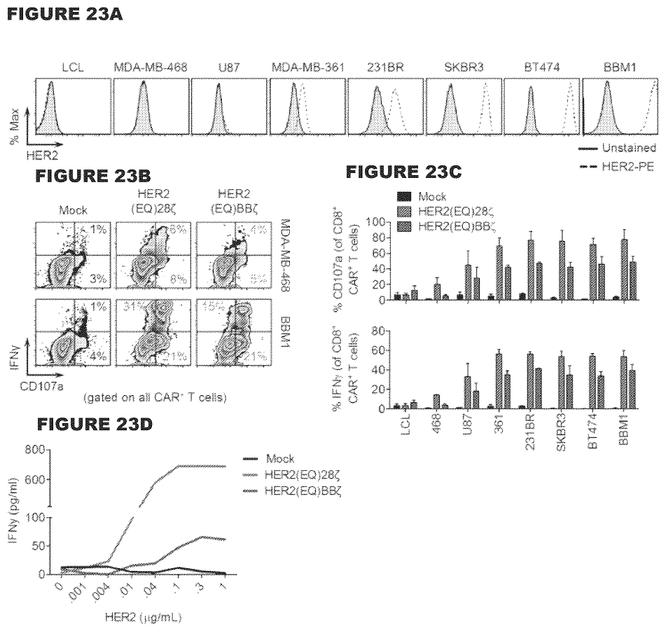

A variety of breast cancer cell lines, including, HER2-negative lines (LCL lymphoma, MDA-MB-468, U87 glioma), low-HER2 expressing lines (MDA-MB-361, 231BR) and high-HER2 expressing lines (SKBR3, BT474, BBM1) were used to characterize HER2(EQ)28ζ and HER2(EQ)BBζ. FIG. 23A depicts the HER2 expression level of each of these lines. Flow cytometry (gated on CAR+ T cells) was used to characterize CD107a degranulation and IFNγ production in Mock (untransduced), HER2(EQ)28ζ or HER2(EQ)BBζ CAR T cells following a 5 hr co-culture with either MDA-MB-361 tumor cells (low HER2 expressing) or BBM1 tumor cells (high HER2 expressing). The results of this analysis are presented in FIG. 23B. Similar studies were conducted with the other breast cancer cells lines, and the results are summarized in FIG. 23C. Production of IFNγ production by HER2-CAR T cells following a 24 hr culture with recombinant HER2 protein or tumor targets was measured by ELISA and the results of this analysis are shown in FIG. 23D.

### Example 12: In Vitro Anti-Tumor Activity of HER2 Targeted CAR

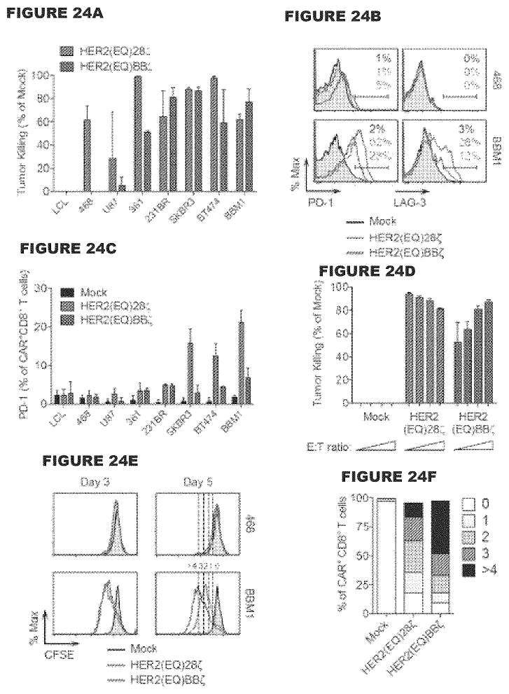

Flow cytometry was used to assess tumor cell killing following a 72 h co-culture of Mock (untransduced), HER2(EQ)28ζ or HER2(EQ)BBζ CAR T cells with tumor targets. The results of this analysis are presented in FIG. 24A. PD-1 and LAG-3 induction in total CAR T cells after a 72 h co-culture with HER2-negative MDA-MB-468 or HER2-positive BBM1 cells was measured, and the results of this analysis are presented in FIG. 24B. PD-1 induction in CD8+ CAR T cells following a 72 h co-culture with tumor targets that are HER2-negative (LCL lymphoma, MDA-MB-468, U87 glioma), low-HER2 expressing (MDA-MB-361, 231BR) or high-HER2 expressing (SKBR3, BT474, BBM1) was measured, and the results of this analysis are presented in FIG. 24C. These studies suggest that HER2(EQ)BBζ causes lower PD-1 induction that does HER2(EQ)28ζ. Tumor cell killing with Effector:Tumor (E:T) ratio ranging from 0.25:1 to 2:1 was measured for both HER2(EQ)28ζ or HER2(EQ)BBζ CAR T cells. The results of this analysis are presented in FIG. 24D, which shows that both HER2(EQ)28ζ and HER2(EQ)BBζ are effective in tumor cell killing in vitro. CFSE proliferation of HER2-CAR T cells following a 72 h co-culture with MDA-MB-468 or BBM1 cells was measured by flow cytometry. The results of this analysis are presented in FIG. 24E, which shows that HER2(EQ)BBζ CAR T cells proliferate more than HER2(EQ)28ζ CAR T cells.

### Example 13: In Vivo Anti-Tumor Activity of HER2 Targeted CAR

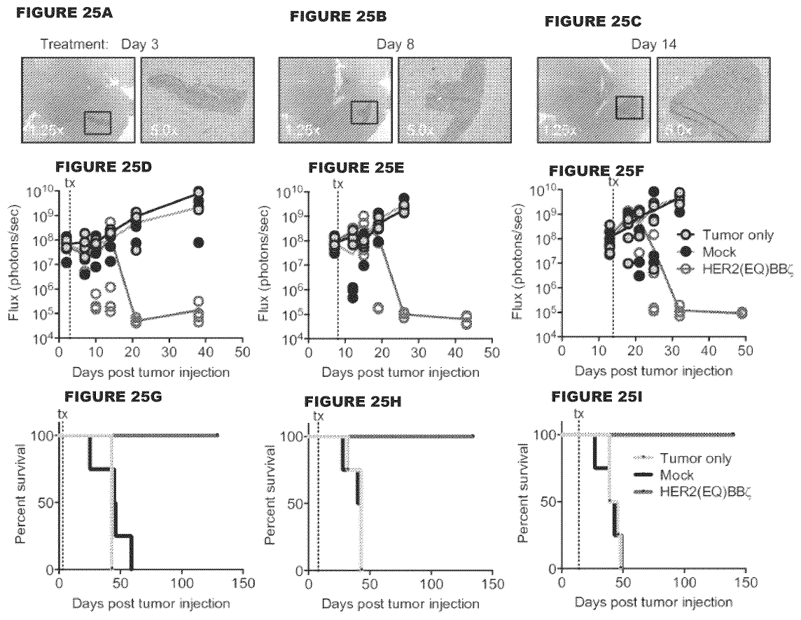

The activity of intratumorally delivered HER2 CAR T cells was assessed in a patient-derived breast-to-brain metastasis model. FIGS. 25A-25C are H&E staining of tumors. Mice were treating by injection directly into the tumor with Mock (untransduced) or HER2(EQ)BBζ CAR T cells. FIGS. 25D-25F depict the results of optical imaging of the tumors and FIGS. 25G-25I are Kaplan-Meier survival curves for mice treated locally with either at day 3, 8 or 14 post tumor injection. These studies show that HER2(EQ)BBζ CAR T cells have potent anti-tumor efficacy in vivo when injected directly into the tumor.

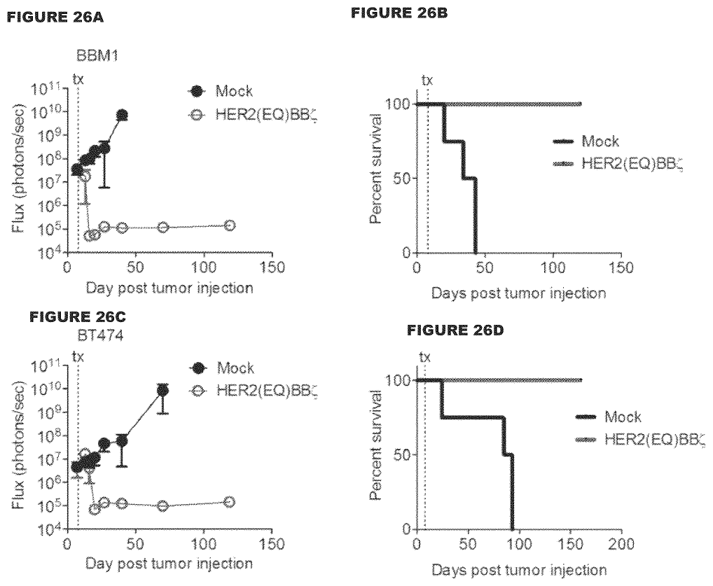

To assess anti-tumor efficacy in human xenograft models of breast-to-brain metastasis, BBM1 cells (0.2M) or BT474 (0.15M) were intracranially injected in NSG mice. At day 8 post tumor injection, HER2(EQ)28ζ or HER2(EQ)BBζ, or Mock (untransduced) T cells (1M) were injected intratumorally. BBM1 (FIG. 26A) and BT474 (FIG. 26B) tumors were monitored by luciferase-based optical imaging. Kaplan Meier curves are presented in FIG. 26C and FIG. 26D.

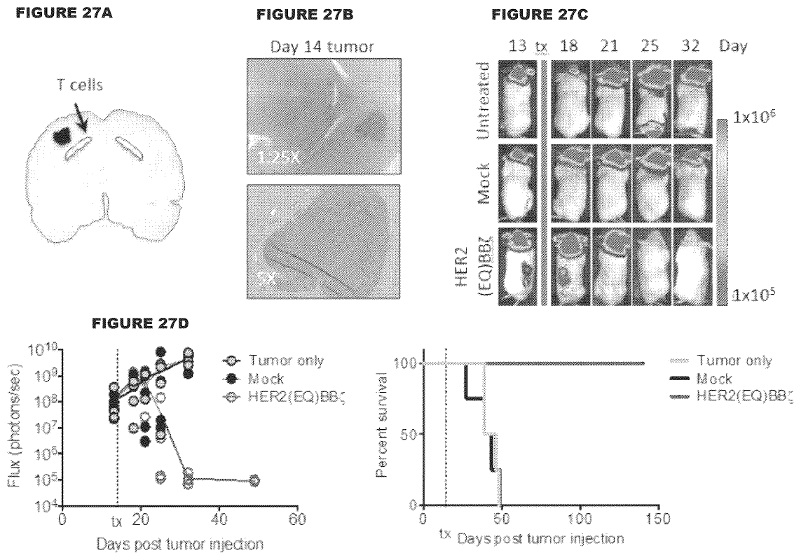

A human patient-derived orthotopic xenograft model of breast-to-brain metastasis was also used to assess HER2(EQ)28ζ and HER2(EQ)BBζ CAR T cells. FIG. 27A illustrates the region of tumor implantation by stereotactic injection of BBM1 cells (0.2M), and intraventricular T cell delivery. Staining of tumors is depicted in FIG. 27B. At day 14 post tumor injection, HER2(EQ)28ζ, HER2(EQ)BBζ, or Mock (unstransduced) T cells (0.5M) were injected intratumorally. Tumor growth was monitored by luciferase-based optical imaging. FIG. 27C presents the flux averages for each treatment group, and FIG. 27D presents the Kaplan Meier survival curve for each treatment group.

### Example 14: Comparison of Intracranial and Intratumoral Administration of T_{CM} Expressing a CAR Targeted to IL13Rα2

Two different intracranial (ic) delivery routes, intratumoral (ict) and intraventricular (icy) were assessed in a murine model of glioblastoma for in vivo safety, trafficking and efficacy of CAR T cells generated from T_{CM}-enriched cell lines that were transduced with the IL13(EQ)BBZ-T2A-CD19t_epHIV7 lentiviral vector and expanded in vitro as proposed for the clinical treatment of glioblastoma (GBM). In vivo safety and functional potency of these cells administered either ict or icy was examined in immunodeficient NSG mice using the IL13Rα2+ primary low-passage GBM tumor sphere line PBT030-2, which has been engineered to express the firefly luciferase (ffLuc) reporter gene.

T_{CM} cell lines that had been enriched from PBMC by CliniMACS™/AutoMACS selection were lenti-transduced with IL13(EQ)BBZ-T2A-CD19t_epHIV7 lentivirus, expanded and then cryopreserved using methods similar to that described above. Freshly thawed CAR T cells administered either ict or icy were then evaluated for potential toxicity, their ability to traffic to multifocal GBM tumors and their potency in controlling the in vivo growth of ic engrafted IL13Rα2+ GBM line PBT030-2 cells. To assess general toxicity, mice were observed daily for overall health, including body weight and alertness. Tumor burden, as measured by Xenogen imaging, was examined; and immunohistochemistry (IHC) to detect T cell recruitment/infiltration of the tumors was also performed on a subset of mice.

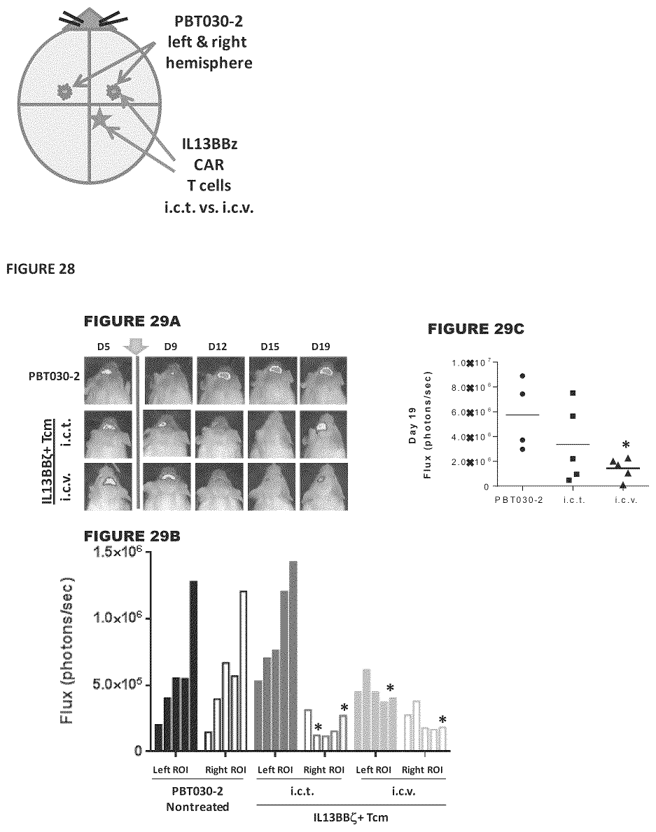

Male NSG mice (10-12 weeks old) were stereotactically injected ic with 1×10⁵ ffLuc+ PBT030-2 cells in both the right and left contralateral hemispheres on day 0 and allowed to engraft for 6 days. Mice were then grouped based on tumor size as determined by Xenogen imaging for equal tumor size distributions per group. Groups of mice were then left untreated (n =4), or treated either ict (right hemisphere, n=8) or icy (n=8) with 1×10⁶ CAR+IL13(EQ)BBζ/CD19t+ T_{CM} (FIG. 28). PBT030-2 tumor growth was monitored over time by Xenogen imaging and quantification of ffLuc flux (photons/sec). At different time points, mice from each group were euthanized, their brains harvested, embedded in paraffin and immunohistochemistry (IHC) was performed to evaluate the presence of human CD3-expressing cells (i.e., human T cells). Specifically, three mice were euthanized from each CAR T cell treated group one week after the T cell administration (Day 13 of the experiment), and thus these mice were not included in the Xenogen imaging analysis of FIG. 29; and then two mice in each of the groups of mice were euthanized two weeks after the T cell administration (Day 21 of the experiment).

While this was not a survival study, and thus mice were all euthanized at specific time points to evaluate T cell trafficking in the brains (described below), the mice were monitored daily for any obvious signs of distress or general toxicity. Mice treated with either the ict or icy regimen did not exhibit any weight loss, and were bright, alert and reactive throughout the experiment. Thus, regardless of the route of T cell administration, there were no signs of any therapy-associated adverse effects.

As shown in FIGS. 29A-C, ict delivery of IL13(EQ)BB/CD19t+ T_{CM} exhibited robust anti-tumor activity against the PBT030-2 tumors as expected. However, icv delivery of IL13(EQ)BBQ/CD19t+ T_{CM} appeared to provide greater therapeutic benefit against ic engrafted PBT030-2 tumors than that observed with ict administration, especially against the tumor lesion in the contralateral (left) hemisphere.

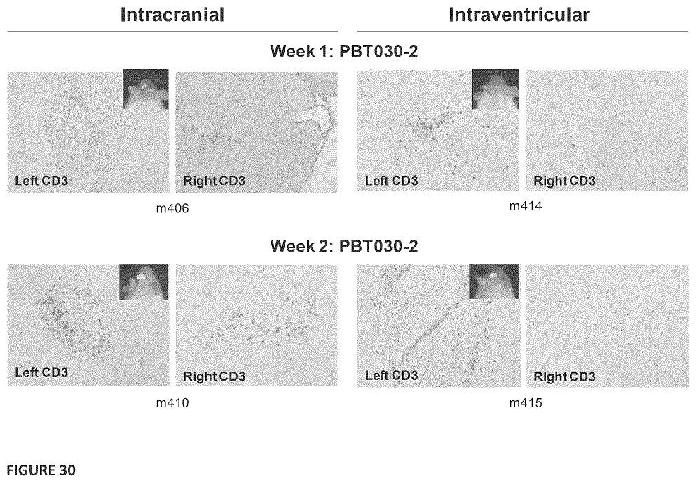

To determine if route of administration affected the ability of T cells to migrate to the tumor site, IHC analysis for CD3+ T cells was performed on the brains of mice from each group at one and two weeks after the T cell administration. As shown in FIG. 30, human CD3+ T cells were found at both the left and right tumor sites in mice that had received either ict or icv administered T cells. These data are representative of 3 mice in each group at one week, and 2 mice in each group at two weeks post T cell administration.

This study demonstrates that both intratumoral and intraventricular administration of T cells were well-tolerated in this NSG mouse model. Furthermore, in vivo multi-focal anti-tumor efficacy and IHC detection of T cells at the tumor sites can be observed with both ict and icv delivery of T_{CM} qualification run cells that had been transduced with the IL13(EQ)BBZ-T2A-CD19t_epHIV7 vector. This study further suggests that icv delivered T cells may have greater efficacy than ict delivered T cells.

### Example 15: Phase 1 Clinical Trial Evaluating IL-13Rα2 CAR T Cells for Treatment of Glioblastoma

This example describes the initial findings of a clinical trial evaluating the safety, feasibility and bioactivity of weekly intracranial infusions of autologous IL13BBζ Tcm in patients with recurrent IL13Rα2+ GBM. As described in greater detail below, Enrolled patients undergo leukapheresis to collect autologous PBMC and, concurrent with IL13BBζ Tcm manufacturing, tumor biopsy or resection is performed, with placement of a reservoir/catheter device. Following baseline MR and PET imaging and recovery from surgery, patients are treated on a 4-week therapeutic regimen, consisting of 3-weekly intracranial infusions of IL13BBζ+ Tcm followed by one rest week for toxicity and disease assessment. The results to date for this first low dose cohort of three resection patients, suggest that local delivery of IL13BBζ Tcm post surgical resection is safe and well-tolerated with no grade 3 or higher toxicities attributed to the therapy observed, and importantly, demonstrate early evidence for antitumor activity following CAR T cell administration. For all patients in which a sample was available, CAR T cells were detected in the tumor cyst fluid or cerebral spinal fluid (CSF) by flow cytometry for a minimum of 7 days post treatment. One patient of particular interest presented with a recurrent multifocal GBM, including one metastatic site in the spine and extensive leptomeningeal disease. This patient was initially treated per protocol with six local infusions of IL13BBζ Tcm into the resection cavity of the largest recurrent tumor focus in the posterior temporal-occipital region. Encouragingly, this CAR T cell injection site remained stable without evidence of disease recurrence for over 7-weeks, while other disease foci distant from the CAR T cell injection site continued to progress. This patient was then treated on a compassionate use protocol with five weekly intraventricular infusions of IL13BBζ Tcm without any other therapeutic interventions. One week following the final intraventricular CAR T cell infusion, all intracranial and spinal tumors had regressed with most decreasing more than 75% by volume, and this patient remained clinically stable four months following the start of CAR T cell treatment.

The CAR, IL13(EQ)BBζ, used in this study is described above. The sequence of the immature CAR, including the CD19t marker is depicted in FIGS. 9A-9B. The entire immature sequence (SEQ ID NO:1) includes: a 22 amino acid GMCSF signal peptide (SEQ ID NO:2), a 112 amino acid IL-13 sequence (SEQ ID NO:3; amino acid substitution E13Y shown in bold); a 229 amino acid IgG4 sequence (SEQ ID NO:4; with amino acid substitutions L235E and N297Q shown in bold); a 22 amino acid CD4 transmembrane sequence (SEQ ID NO:5); a 42 amino acid 4-1BB sequence (SEQ ID NO:6); a 3 amino acid Gly linker; a 112 amino acid CD3ζ sequence (SEQ ID NO:7); a 24 amino acid T2A sequence (SEQ ID NO:8); and a 323 amino acid CD19t sequence (SEQ ID NO:9).

Autologous cells from each patient was used to prepare CD8+ CD4+ T_{CM} cells which were then transfected with a lentiviral vector, described above, expressing IL13(EQ)BBζ. Briefly, T_{CM} were enriched from peripheral blood mononuclear cells (PBMC) using the CliniMACS® device to immunomagnetically select for CD45RO+/CD62L+ T_{CM}. These cells were activated with anti-CD3/CD28 Dynal beads, transduced with a SIN lentiviral vector that directs the expression of the IL13(EQ)BBζ CAR. The activated/genetically modified IL13(EQ)BBζ/CD19t+ T_{CM} cells were expanded in vitro with IL-2/IL-15 and then cryopreserved.

The treatment of two patients, both suffering from relapsed/refractory GBM is described below. Intracavity administration of CAR T cells was performed manually over about 5-10 minutes through a Rickham catheter followed by up to 1.0 mL preservative-free normal-saline (PFNS) flush delivered by convection enhanced delivery (CED) at 0.5 ml/hour. Intraventricular administration of CAR T cells was performed manually over approximately 5-10 minutes through a Rickham catheter placed into the lateral ventricle. This was followed by up to 0.5 mL preservative-free normal-saline (PFNS) flush delivered via a manual push technique over 5-10 minutes. The PFNS flush is meant to clear the administration line and push remaining CAR T cells through the catheter.

The time course of CAR T cell preparation and treatment is depicted in FIG. 31. Concurrent with the manufacturing process, research participants underwent resection of their tumor(s) followed by placement of a Rickham catheter and baseline imaging.

Patient UPN097 underwent tumor resection and was treated in Cycle 1 with 2×10⁶ cells and in Cycle 2 with 10×10⁶ cells. In both Cycle 1 and Cycle 2 the cells were administered to the cavity left by resection. After the second cycle Patient UPN097 was taken off the study due to rapid tumor progression.

Patient UPN109 was treated in Cycle 1 with 2×10⁶ cells and in Cycles 2 and 3 with 10×10⁶ cells. After a rest period, Patient UPN109 was treated in Cycles 4, 5 and 6 with 10×10⁶ cells. In Cycles 1-6 the cells were administered into. In Cycle 7 the patient was treated with 2×10⁶ cells. In Cycles 8 and 9 the patient was treated with 10×10⁶ cells. In Cycles 7-10 the administration was intraventricular.

FIG. 32A presents analysis of CAR T cell persistence, as monitored by CD19. This analysis shows good T cell persistence 8 days after the Cycle 2. FIG. 32B shows decreased presence of GBM cells as monitored by IL13Rα2 expression on cells.

FIG. 33A and FIG. 33B depict imaging results from Patient UPN097 in the region of the catheter used for intratumoral administration. In FIG. 33A one can see that few CD3+ or CD8+ T cells are present pretreatment. FIG. 33B, which is a sample at Day 16 post-treatment taken from the left frontal tumor cavity wall shows a large area of necrotic tumor and significant presence of CD3+ and CD8+ cells.

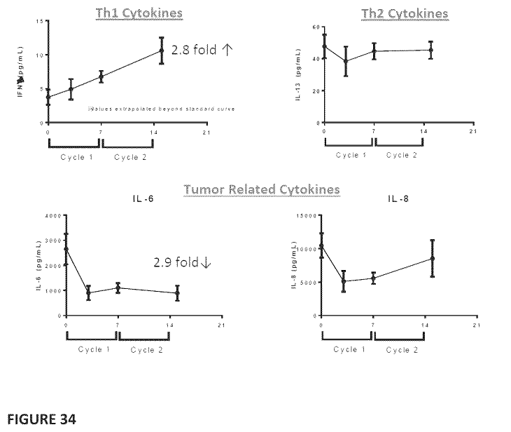

As shown in FIGS. 34A-D, there was an increase in IFN-gamma (a Th1 cytokine) over the two Cycles of treatment while levels of IL-13 (a Th2 cytokine did not change significantly (FIGS. 34A and 34B). IL-6, a tumor related cytokine, decreased during Cycle 1 and remained at the lower level during Cycle 2 (FIG. 34C). IL-8, another tumor related cytokine, decreased during Cycle 1, but increased towards its pre-Cycle 1 level during Cycle 2 (FIG. 34D).

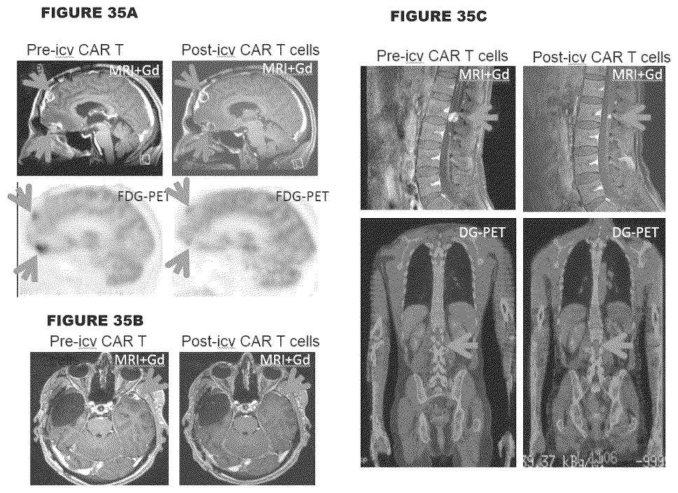

Patient UPN109 presented with a recurrent multifocal GBM, including one metastatic site in the spine and extensive leptomeningeal disease. As described above, this patient was treated with six local infusions of IL13BBζ Tcm into the resection cavity of the largest recurrent tumor focus. While the CAR T cell injection site remained stable without evidence of disease recurrence for over 7-weeks, other disease foci distant from the CAR T cell injection site continued to progress (data not shown). This patient then received five weekly intraventricular infusions of IL13BBζ Tcm, as described above. FIGS. 35A-B presents MRI and/or PET images of transverse brain section (FIG. 35A) and saggital brain section (FIG. 35B). FIG. 35C presents transverse (top) and frontal (bottom) sections of the spine before (left) and one week after (right) completion of intraventricular therapy, with tumor lesion sites indicated by red arrows in each image.

### Example 16: Case Report on Intraventricular Administration

A 50 year old male was initially diagnosed with a low-grade brain tumor in the right temporal lobe after presenting with grand mal seizures. After four months of monitoring, this right temporal tumor displayed increased enhancement by MRI, and the patient underwent tumor resection which confirmed a diagnosis of WHO grade IV glioma (GBM). The patient then received standard-of-care adjuvant proton radiation to a total dose of 59.4 cobalt Gy equivalent with concurrent temozolomide (140 mg daily), followed by 4-cycles of temozolomide with concomitant use of the Novocure device (NovoTTF-100A) for three months. Six months after the primary tumor resection, PET and MRI images showed evidence of disease progression.

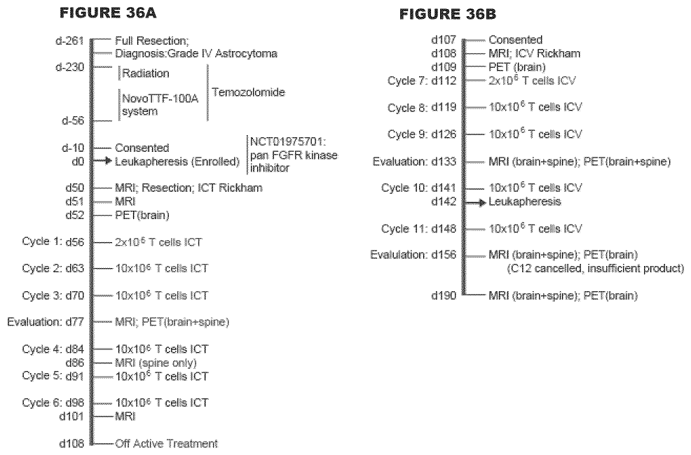

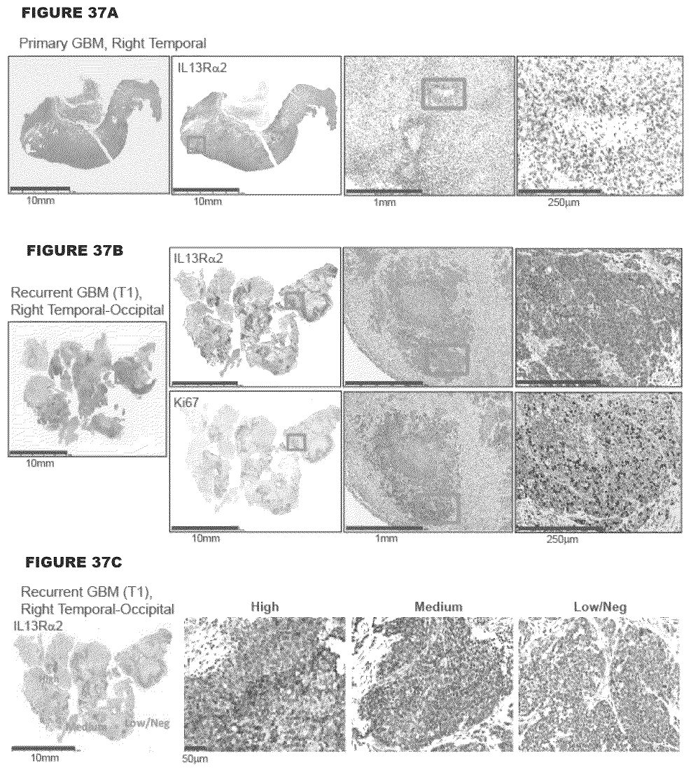

The patient was then treated autologous IL13Rα2-targeted CAR T cells (FIG. 36) following confirmation of IL13Rα2-expression in the primary right temporal lobe tumor by IHC, with an H score of 100 (FIG. 37). Peripheral blood mononuclear cells (PBMC) were then collected by leukapheresis followed by enrichment of CD4+ CD8+ TCM via a two-step depletion selection procedure as previously described. During IL13BBζ TCM manufacturing, the patient was treated on a separate clinical protocol evaluating a fibroblastic growth factor inhibitor (NCT01975701), and progressed through therapy with symptoms of headaches, confusion and disorientation increasing. Additionally, the patient was tapered off steroids prior to T cell injections.

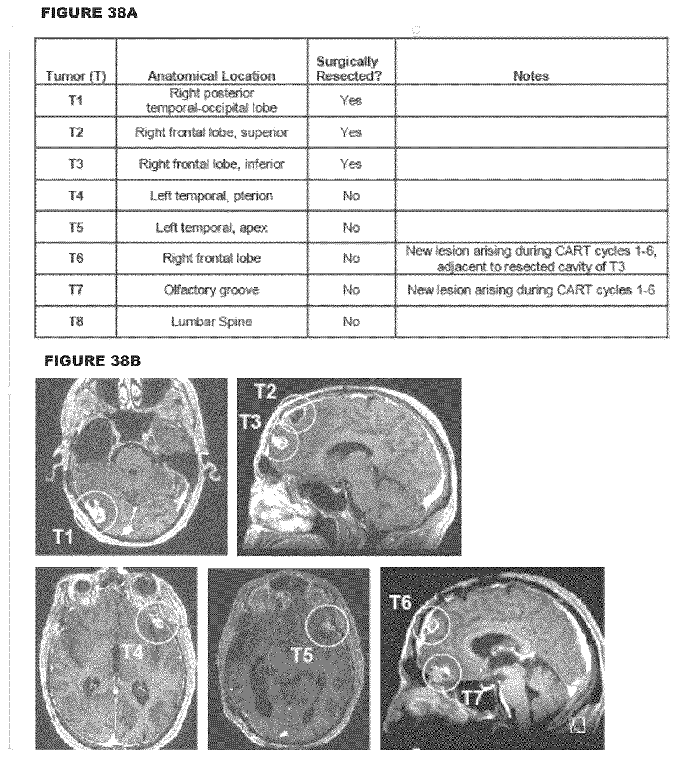

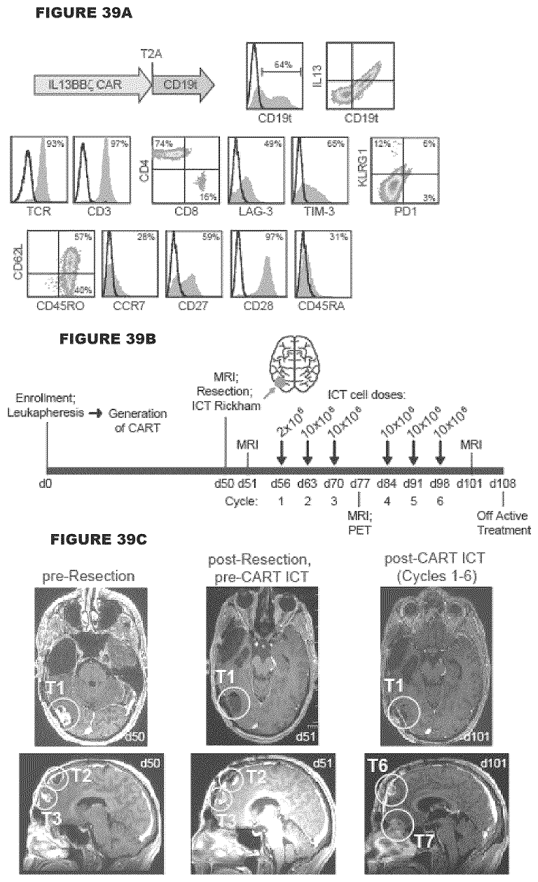

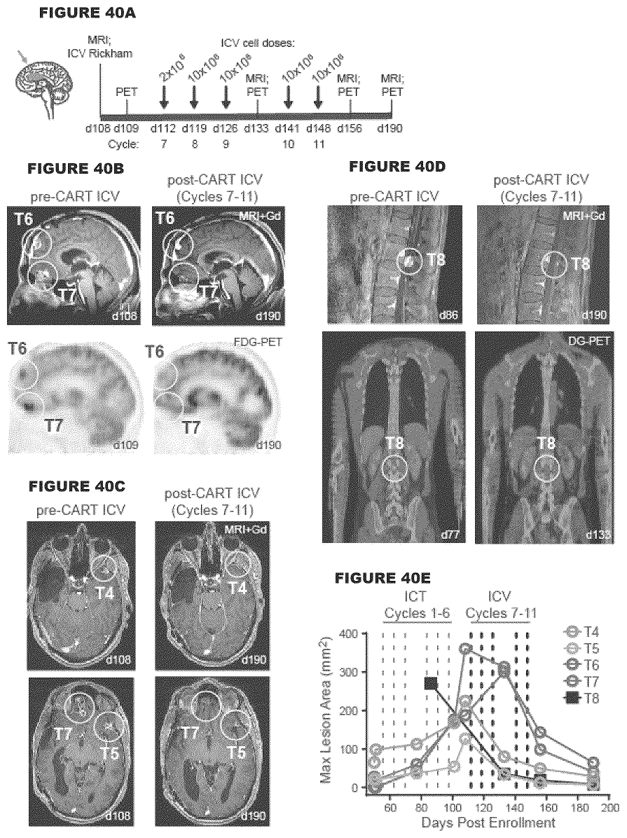

Ten months post primary tumor resection, the patient underwent another surgical resection for three of five identified progressing GBM lesions (FIG. 38), including the largest lesion in the right posterior temporal-occipital region (T1) where the reservoir/catheter device was placed, and two lesions in the right frontal lobe (T2, T3). Two additional tumors in the left temporal area were not surgically removed (T4,T5). Six days post-surgical resection, the patient received two 4-week treatment regimens, each consisting of three weekly intracavitary (ICT) infusions of IL13BBζ TCM followed by a week for toxicity evaluation and disease assessment. This patient was treated starting with a low dose of 2×10⁶ CAR+ T cells followed by five infusions of 10×10⁶ CAR+ T cells (FIG. 39). Following these six ICT infusions, under a compassionate use protocol, a second catheter was placed in the right lateral ventricle, allowing the patient to receive an additional five intracerebroventricular (ICV) treatment cycles of IL13BBζ TCM, again starting at a low dose of 2×10⁶ CAR T cells followed by four infusions of 10×10⁶ CAR T cells (FIG. 40).

Due to limited therapeutic product only five ICV infusion cycles were feasible. Overall, the patient received 11 cell infusions for a total dose of 94×10⁶ CAR+ T cells. The treatment course encompassed 15-weeks, with evaluation weeks for toxicity and disease assessment (i.e., MRI and PET imaging) taking place after every third cycle, and after the final two ICV infusions. The patient received no other therapeutic interventions during this CAR T cell treatment course, and findings up to the 190 day evaluation period, encompassing the 11 infusions cycles, is reported here. Subsequently, a second IL13BBζ TCM product was manufactured and beginning on day 192 this patient has continued to receive ICV infusions of this second manufactured product approximately every 3 weeks.

### Example 17: Study Design

These studies, including the compassionate use protocol, were approved by an institutional review board, and the patient provided written informed consent. Eligibility included prior histologically-confirmed diagnosis of an IL13Rα2+ grade IV glioma that is now recurrent, age >18 years with a Karnofsky performance status (KPS) >60, adequate cardiopulmonary function, and a survival expectation >4 weeks. The patient must have completed initial radiation therapy at least 12 weeks prior to enrollment, and must not have any other active malignancies, infections or intercurrent illness or be receiving other investigational agents or require more than 2 mg TID (3×/day) of Dexamethasone during T cell therapy.

This patient was initially treated under our ongoing phase I study (NCT02208362) to evaluate the safety and feasibility of weekly intracranial infusions of autologous IL13Rα2-targeted CAR T cells (IL13BBζ TCM) in patients diagnosed with recurrent/refractory IL13Rα2+ high-grade glioma (WHO Grades III and IV). This is a two arm study with T cells administered either directly into the tumor (stratum 1=intratumoral) or into the tumor resection cavity (stratum 2=intracavitary). After completing the six intracavitary (ICT) infusion cycles, this patient was then treated under a separate compassionate use protocol allowing for intracerebroventricular (ICV) delivery of IL13BBζ TCM.

### Example 18: Cell Product Manufacture and Infusion

The lentiviral vector encoding the 4-1BB costimulatory, IL13Rα2-targeted CAR, IL13BBζ, is detailed herein. Briefly, the codon optimized CAR sequence contains a membrane-tethered human IL-13 ligand mutated at a single site (E13Y) to reduce potential binding to IL13Rα1, a human IgG4 Fc spacer containing two mutations (L235E; N297Q) that prevent Fc receptor-mediated recognition, a human CD4 transmembrane domain, a human costimulatory 4-1BB cytoplasmic signaling domain, and a human CD3ζ cytoplasmic signaling domain. A T2A ribosome skip sequence then separates this IL13BBζ CAR sequence from a truncated human CD19 sequence (CD19t), an inert, nonimmunogenic cell surface marker.

For IL13BBζ TCM manufacturing, on the day of leukapheresis, PBMC were isolated by density gradient centrifugation over Ficoll-Paque (GE Healthcare) followed by two washes in PBS/EDTA. PBMC were then washed once in PBS, resuspended in X Vivo15 media (Bio Whittaker) containing 10% fetal calf serum (FCS) (Hyclone), transferred to a 300 cc transfer bag, and stored on a 3-D rotator overnight at room temperature (RT). The following day, 5×109 PBMC were incubated in a 300 cc transfer bag with clinical grade anti-CD14 (1.25 mL), anti-CD25 (2.5 mL) and anti-CD45RA (2.5 mL) microbeads (Miltenyi Biotec) for 30 minutes at RT in X Vivo15 containing 10% FCS. CD14+, CD25+, and CD45RA+ cells were then immediately depleted using the CliniMACS™ depletion mode according to the manufacturer's instructions (Miltenyi Biotec). After centrifugation, the unlabeled negative fraction of cells was resuspended in CliniMACS™ PBS/EDTA buffer (Miltenyi Biotec) containing 0.5% human serum albumin (HSA) (CSL Behring) and then labeled with clinical grade biotinylated-DREG56 mAb (COHNMC CBG) at 0.6 mL for 30 minutes at RT. The cells were then washed and resuspended in a final volume of 100 mL CliniMACS™ PBS/EDTA containing 0.5% HSA and transferred into a new 300 cc transfer bag. After 30 minutes incubation with 1.25 mL anti-biotin microbeads (Miltenyi Biotec), the CD62L+ fraction (TCM) was purified with positive selection on CliniMACS™ according to the manufacturer's instructions, and resuspended in X Vivo15 containing 10% FCS.

Within 2 hours of enrichment, 26.9×10⁶ TCM were stimulated with GMP Dynabeads® Human T expander CD3/CD28 (Invitrogen) at a 1:3 ratio (T cell:bead), and transduced with clinical grade IL13BBζ-T2A-CD19t_epHIV7 at an MOI of 0.3 in 5.5 mL×Vivo15 containing 10% FCS with 5 g/mL protamine sulfate (APP Pharmaceutical), 50 U/mL rhIL-2 and 0.5 ng/mL rhIL-15 in a 32 Vuelife tissue culture bag (AFC) that was placed at a horizontal position on a culture rack at 37° C., 5% CO2. Cultures were then maintained with addition of X-Vivo15 10% FCS as required to keep cell density between 4×10⁵ and 2×10⁶ viable cells/mL, with cytokine supplementation (final concentration of 50 U/mL rhIL-2 and 0.5 ng/mL rhIL-15) every Monday, Wednesday and Friday of culture. Based on culture volume, T cells were transferred to 730 Vuelife bags (AFC). Seven days after the lentiviral transduction, the CD3/CD28 Dynabeads were removed using the Dynal ClinEx Vivo Magnetic Particle Concentrator bag magnet, and bead-free T cells were drained into a new 730 Vuelife bag. Cultures were propagated until approximately 4.53×10⁸ cells were generated as determined by Guava PCA, at which time cultures were harvested, washed in Isolyte (Braun) with 2% HSA, then resuspended in Cryostor CS5 (BioLife Solutions) at approximately 1.3×10⁷ cells/mL for cryopreservation using a Mr. Frosty (Nalgene) and a portable controlled rate freezer system (Custom Biogenics). Quality control tests included viability, potency (CD19t expression), Identity (CD3 expression), transgene copy number (WPRE qPCR), replication competent virus testing (VSV-G qPCR and formal RCL testing at the University of Indiana), residual bead count, and sterility.

As noted above, the T2A ribosome skip sequence1212 then separates this IL13BBζ sequence from CD19t, an inert, nonimmunogenic cell surface marker marking cell transduction (FIG. 39A). This T2A linkage results in the coordinate expression of both IL13BBζ and CD19t from a single transcript.

Manufacturing methods for the immunomagenetic enrichment of CD62L+ CD45RA− CD4+ CD8+ central memory T cells (TCM), lentiviral transduction and ex vivo expansion are also detailed herein. The end-of-process (EOP) cyropreserved IL13BBζ TCM product underwent quality control release testing as per the clinical protocol. For each infusion, T cells were thawed, washed and reformulated into a final volume of 0.5 mL in pharmaceutical preservative-free normal saline (PFNS) with 2% human serum albumin (HSA). Cells were manually injected into the Rickham reservoir using a 21 gauge butterfly needle to deliver a 0.5 mL volume over 5-10 minutes, followed by up to 1 mL PFNS flush delivered by convection enhanced delivery (CED) at 0.5 mL per hour.

### Example 19: Clinical Imaging

The post-gadolinium T1 weighted MRI sequences of the brain and spine were acquired on a Siemens Viro 3 Tesla scanner. Lesions were measured on axial T1 MPR weighted images obtained after the administration of Multihance. Imaging with 18-F-fluorodeoxyglucose (18-F-FDG) was performed using a GE Discovery DST HP60 PET-CT scanner (70 cm axial field of view, slice thickness 3.75 mm). Maximal standardized uptake values (SUVs) were obtained utilizing Vital Images Vitrea version 6.7.2 software. Regions of contrast-enhancing tumor foci were outlined by a radiologist for measurements of largest tumor area (mm2) and tumor volumes (cm3) were computed.

Cryopreserved cell banks of quality control released autologous IL13BBζ TCM were thawed and reformulated for infusion by washing twice with phosphate buffered saline (PBS) with 2% HSA and resuspending in pharmaceutical preservative-free normal saline with 2% HSA. Delivery of the therapeutic CAR T cells into either the glioma resection cavity (ICT) or the lateral ventricle (ICV) was achieved using a Holter™ Rickham Ventriculostomy Reservoir (Codman), with a ventricular catheter (Integra Pudenz), and a stylet. For ICT delivery, the reservoir/catheter system was inserted at the time of tumor resection, and the tip of the catheter was partially embedded into the resection wall in order to allow for cell delivery both into the cavity and into the peritumoral brain tissue. Post-operative imaging (CT and MRI) were obtained to confirm catheter position and extent of tumor resection.

### Example 20: IL13BBC TCM Display a Central Memory-Like T Cell Phenotype

Enriched TCM (36×10⁶) were ex vivo stimulated, lentivirally transduced and expanded to yield 638×10⁶ total cells in 17 days. The final T cell product (CD3+ and TCR+) consisted of CD4 (74%) and CD8 (16%) T cell subsets and expressed IL13BBζ and CD19t with gene modified co-staining for both cell surface proteins (FIG. 39A). The CAR T cell product exhibited a central memory T cell phenotype, expressing CD45RO (97%), CD62L (57%), CCR7 (28%), CD28 (97%) and CD27 (59%) (FIG. 39A). The product also expressed of some markers of exhaustion, including TIM-3 (65%) and LAG-3 (49%), but not significant levels of PD-1 and KLRG1 (FIG. 39A).

### Example 21: Safety and Tolerability of Repetitive Intracranial Infustions of IL13BBζ TCM

The patient was treated with weekly infusions of IL13BBζ TCM administered via a reservoir/catheter device through two different intracranial delivery routes, that being intracavitary (ICT) delivery following tumor resection (cycles 1-6) and intracerebroventricular (ICV) delivery into the cerebral spinal fluid (CSF) (cycles 7-11). The 11 intracranial infusions, at a maximum cell dose of 10×10⁶ CAR+ T cells, were well-tolerated with no grade 3 or higher toxicity (NCI Common Toxicity Criteria) with possible or higher attribution to the therapy observed. Mild events noted following CAR T cell infusions include.

TABLE 6
SAFETY AND TOLERABILITY

| | | Maximum | | |
|---|---|---|---|---|
| Delivery | T-cell | T-cell | Cumulative | Adverse Event |
| Route | Doses | Dose | T-cell Dose | (Grade 1-2)* |
| ICT | 6 | 10⁷ | 5.2 × 10⁷ | Chills |
| | | | | Fatigue |
| | | | | Fever |
| | | | | Lymphopenia |
| | | | | Myalgia |
| | | | | Dizziness |
| | | | | Headache |
| | | | | Seizure |
| ICV | 5 | 10⁷ | 4.2 × 10⁷ | Chills |
| | | | | Fatigue |
| | | | | Fever |
| | | | | Myalgia |
| | | | | Headache |
| | | | | Short Olfactory Aura |
| | | | | Seizure |
| | | | | Anxiety |
| | | | | Hypertension |
| | | | | Sinus Tachycardia |

*Only events with possible or higher attribution to the T cell administration are reported; all occurred once and were Grade 1-2 according to the NCI Common Toxicity Criteria, with no events of Grade 3 or higher observed.

### Example 22: Clinical Response

At the time of treatment, the patient's tumor displayed characteristics of a highly aggressive recurrent GBM with poor prognostic features. This included evidence of recurrence from primary diagnosis within six months following standard-of-care therapy, the presentation of multifocal tumor lesions, including spinal lesions and extensive leptomeningeal disease (FIG. 38), histological features of a dedifferentiated GBM, and a high tumor proliferation rate with over 60% of the cells staining positive for Ki67 (FIG. 37B). Tumor expression of IL13Rα2, as evaluated by IHC on FFPE of resected tumor tissue, was similar between the primary and recurrent tumors with an H score of 100 and 80, respectively (FIG. 37). Intratumoral IL13Rα2 expression for the recurrent tumor was heterogeneous, with 10% of the cells showing high staining intensity (2-3+), 60% showing low expression (1+), and 30% of the cells not staining above background (0+). Of potential interest, the highest levels of IL13Rα2 expression were often observed in tumor regions of pseudopalisading necrosis (FIG. 37A), an expression pattern noted for other GBM tumors.

Following enrollment on the clinical protocol, this patient underwent surgical resection for three of the five recurrent lesions (FIG. 38), and the reservoir/catheter device was place in the resected cavity of the largest recurrent foci (T1) in the right posterior temporal-occipital area. This patient was initially treated per protocol with six weekly intracavitary (ICT) infusions of CAR+ T cells (2×10⁶ cycle 1; 10×10⁶ cycles 2-6) (FIG. 1B), and during this time period the temporal-occipital tumor lesion (T1) remained stable for over 45-days post-surgery without evidence of progressive disease (FIG. 39C). MRI revealed, however, that other non-resected tumor foci in the left temporal lobes (T4 and T5), as well as a new recurrent lesion adjacent to tumor 3 (T6) and a lesion in the olfactory groove (T7) continued to progress over this same time period (FIG. 39C). Additionally, metastatic lesions in the spine, including one large tumor (270 mm²) and more than one small tumor foci (<1 cm²) were also detected. These results, while mixed, were encouraging, in that they suggested the IL13BBζ T_{CM} may have prevented disease recurrence at the resected posterior temporal-occipital area, however, they also suggested that local ICT delivery was not sufficient to effectively control tumor progression at distant locations away from the infusion site.

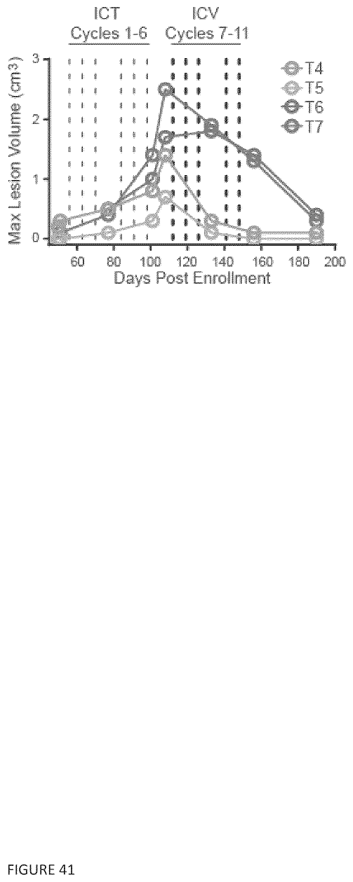

Based on these findings and supported by preclinical studies showing that ICV delivery of CAR T cells can traffic to multifocal GBM in NSG mouse models (data not shown), this patient was enrolled on a compassionate use protocol and treated with five weekly ICV infusions of IL13BBζ T_{CM} without any other therapeutic interventions (2×10⁶ cycle 7; 10×10⁶ cycles 8-11) (FIG. 40A). One week after three ICV infusions (cycle 9; day 133) all tumor lesions showed dramatic regression, and following the final ICV infusion (cycle 11; day 156), most intracranial and spinal tumors had regressed more than 70% by both maximum area and volume measurements (FIG. 40B-E, Table 7 below, and FIG. 41). Follow-up MR and PET imaging six weeks after the last ICV infusion (day 190), during which the patient received no other therapeutic intervention, showed continued disease regression, with all tumors decreasing ≥78% by both maximum area and volume measurements (FIG. 40B-E, Table 7 below, and FIG. 41). At this 190 day time point, it was not possible to differentiate between residual radiographic evidence of disease versus inflammation, scaring and/or dural enhancement. ICV delivery of IL13BBC T_{CM} elicited almost complete elimination of all metastatic tumors in the spine, with 97% reduction in the maximum area for the largest lesion and only one small tumor foci visible out of the more than ten present prior to ICV treatment. Over the time course of ICV treatment, and coinciding with tumor regression, the patient was able to reduce system dexamethosome from 2 mg bid to 0.5 mg qd. This patient remains clinically stable and has returned to normal life and work activities, thus supporting the durability of this CAR T cell-mediated antitumor response. These results demonstrate that treatment with IL13BBζ T_{CM} mediated a near complete response based on the stringent RANO criteria.

TABLE 7
MRI Evaluation of Non-Resected Lesions (Volume in cm³, Area in mm²)

| | | | | Post | | | | Post | | | |
|---|---|---|---|---|---|---|---|---|---|---|---|
| | | | Post Op | Cycles | | | Post Op | Cycles | | | |
| | Anatomical | Pre Op | i.c.t. | 1-3 | Post Cycles 4-6 | | i.c.v. | 7-9 | Post Cycles 10-11 | | Max % |
| Tumor | Location | D50 | D51 | D77 | D86 | D101 | D108 | D133 | D156 | D190 | Decrease |
| 4 | Left | 0.2 cm³ | 0.3 cm³ | 0.5 cm³ | ND | 0.8 cm³ | 1.4 cm³ | 0.3 cm³ | 0.1 cm³ | 0.1 cm³ | Vol: 93% |
| | temporal, | 65 mm² | 98 mm² | 112 mm² | | 168 mm² | 224 mm² | 80 mm² | 49 mm² | 28 mm² | Area: 88% |
| | pterion | | | | | | | | | | |
| 5 | Left | 0 cm³ | 0 cm³ | 0.1 cm³ | ND | 0.3 cm³ | 0.7 cm³ | 0.1 cm³ | 0 cm³ | 0 cm³ | Vol: 100% |
| | temporal, | 20 mm² | 20 mm² | 36 mm² | | 54 mm² | 126 mm² | 33 mm² | 11 mm² | 7 mm² | Area: 94% |
| | apex | | | | | | | | | | |
| 6* | Right | NA | NA | 0.5 cm³ | ND | 1 cm³ | 1.7 cm³ | 1.8 cm³ | 1.4 cm³ | 0.4 cm³ | Vol: 78% |
| | frontal lobe | 0 mm² | 0 mm² | 42 mm² | | 176 mm² | 187 mm² | 300 mm² | 143 mm² | 64 mm² | Area: 79% |
| 7* | Olfactory | NA | 0.1 cm³ | 0.4 cm³ | ND | 1.4 cm³ | 2.5 cm³ | 1.9 cm³ | 1.3 cm³ | 0.3 cm³ | Vol: 88% |
| | groove | 27 mm² | 18 mm² | 60 mm² | | 171 mm² | 360 mm² | 312 mm² | 98 mm² | 40 mm² | Area: 89% |
| 8 | Spinal | ND | ND | ND | 270 mm² | ND | ND | 35 mm² | 18 mm² | 8 mm² | Area: 97% |

*new lesion arising during Cycles 1-6
Bold, values compared for Maximum % Decrease
NA, no lesion could be identified
0, lesion might be visually identified, but value was below that of analysis software parameters
ND, imaging was not done

### Example 23: CAR T Cell Persistence and CNS Inflammatory Response

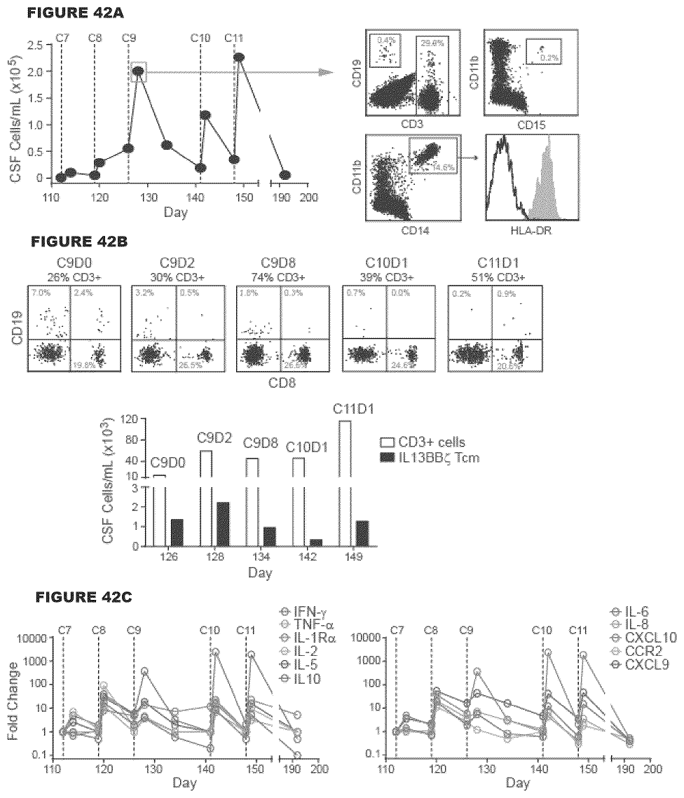

To elucidate immunological changes associated with antitumor responses observed following the ICV infusion of IL3BB T_{CM} (Cycles 6-11), CSF was evaluated for cell infiltrates, CAR+ T cell persistence, and cytokine levels. Immediately following each ICV infusion (i.e., day 1-2 of cycles 6-11), cell numbers per mL of CSF increased 7.0±3.6 fold as compared to pre-infusion levels (day 0 of each cycle), and increased 153±128 fold as compared to pre-ICV (C7D0) levels (FIG. 42). Total cell numbers in the CSF typically decreased over the 7-day treatment cycle. As evaluated for C9D2, the cell infiltrates included a large proportion of CD3+ T cells, both endogenous and CAR-expressing, as well as CD14+ CD11b+ HLA-DR+ mature myeloid populations (FIG. 42A). Only rare CD19+ B cells and CD11b+ CD15+ granulocytes were detected (FIG. 42A). Consistent with flow cytometry data, CSF cytopathology on C11D1 reported the presence of reactive lymphocytes, monocytes, and macrophages.

CAR-T persistence was also monitored over the ICV treatment course. Due to low cell recovery in the CSF for cycles 7 and 8, analysis focused on evaluating cycle 9 and time points immediately following cycles 10 and 11. Importantly, CAR+ T cells were detected at all-time points evaluated (FIG. 42B), including C9D0 which corresponded to 7-days post cycle 8, thus demonstrating persistence of the therapeutic cells for at least 7 to 8 days post-infusion. However, CAR T numbers in the CSF post infusion decreased at the later cycles (C10D1 and C11D1) when tumor burden had also significantly decreased (FIG. 40B). To note, significant expansion of the CAR T cells in the CSF over cycle 9 was not detected, with CAR+ cell numbers increasing 1.6-fold 2 days later (C9D2) from pre-infusion (C9D0) and then decreasing 2.3-fold by day 8 (C9D8).

The presence of immune cells, including CAR+ T cells, following each infusion corresponded to significant elevations of cytokine levels in the CSF. The measured levels and calculated fold-change over baseline for the 30-cytokines measured is presented in Tables 9 and 10 below. Notably, 11 cytokines increased more than 10-fold from pre-ICV baseline (C7D0) immediately following IL3BBC T_{CM} infusions, including cytokines IFNγ, TNF, IL-2, IL-10, IL-5, IL-6, and IL-8 and chemokines CXCL9/MIG, CXCL10/IP-10, and CCR2/MCP-1 and soluble cytokine receptor IL-1Rα(FIG. 3C). Seven other cytokines showed greater than a 5-fold increase from baseline (C7D0), including G-CSF, IL-12, IL2-R, IL-4, IL-7, and MIP-lb. The inflammatory cytokines that exhibited the highest fold increase immediately following IL3BBC T_{CM} infusion as compared to pre-ICV (C7D0) was IL-2 (>90-fold for C9D2) and the IFN-γ inducible chemokines CXCL9 and CXCL10 (>40-fold for C8D1, C9D2, C10D1 and C11D2). These cytokines returned to near baseline levels within 7-days between treatment cycles. Cytokines that did not show significant increase following CAR T cell infusions include IL-13, RANTES and VEGF (Tables 9 and 10).

TABLE 9
UPN 109 CSF Cytokine Analysis (pg/mL), ICV Cycles 7 through 11.

| Cytokine | C7D0 | C7D2 | C8D0 | C8D1 | C9D0 | C9D2 | C9D8 | C10D0 | C10D1 | C11D0 | C11D1 | C11D44 |
|---|---|---|---|---|---|---|---|---|---|---|---|---|
| EGF | OOR< | OOR< | OOR< | OOR< | OOR< | OOR< | OOR< | OOR< | OOR< | OOR< | OOR< | 10.0 |
| Eotaxin | *2.1 | *2.4 | *2.4 | 6.2 | 3.0 | 3.8 | *2.4 | *2.4 | 6.3 | *2.4 | 6.1 | *2.6 |
| FGF | 7.1 | 8.0 | 8.8 | 14.1 | *4.0 | *3.1 | *4.7 | *6.3 | 12.2 | 7.3 | 13.6 | 11.5 |
| G-CSF | *25.1 | 68.6 | *43.7 | 232.6 | 103.9 | 137.5 | 64.9 | *23.3 | 245.6 | *13.8 | 248.9 | 39.9 |
| GM-CSF | *2.0 | *2.4 | *2.5 | *8.7 | *2.5 | *4.2 | *1.4 | *1.3 | *3.2 | *1.3 | *2.7 | OOR< |
| HGF | 74.4 | 113.1 | 127.7 | 253.9 | 162.3 | 250.8 | 145.8 | 110.9 | 213.6 | 125.2 | 241.8 | 81.0 |
| IFN-α | 45.5 | 56.8 | 42.0 | 109.7 | 59.5 | 66.1 | 35.6 | 17.9 | 90.0 | 24.8 | 74.5 | OOR< |
| IFN-γ | *8.2 | *7.0 | *3.8 | 140.8 | 16.8 | 32.1 | *5.0 | *1.8 | 69.5 | *4.0 | 42.8 | *1.0 |
| IL-10 | *4.4 | *6.0 | *2.1 | 74.6 | *20.7 | 70.4 | *16.0 | *3.5 | 147.1 | *6.9 | 167.5 | OOR< |
| IL-12 | 16.7 | 23.5 | 24.7 | 92.4 | 41.5 | 82.7 | 62.4 | 35.5 | 57.0 | 42.6 | 85.7 | 12.7 |
| IL-13 | *15.8 | *15.3 | *13.1 | 29.9 | *15.9 | 18.1 | *4.8 | OOR< | 22.7 | OOR< | 18.8 | OOR< |
| IL-15 | OOR< | OOR< | OOR< | OOR< | OOR< | OOR< | OOR< | OOR< | OOR< | OOR< | OOR< | *7.1 |
| IL-17 | *2.4 | *2.8 | *0.9 | *9.2 | *5.4 | *5.1 | *2.3 | *1.2 | *8.5 | *1.0 | *8.1 | OOR< |
| IL-1Rα | *50.1 | *35.7 | *56.9 | 405.9 | 238.6 | 699.3 | 358.0 | 605.2 | 1113.0 | *53.4 | 1141.9 | 259.9 |
| IL-1β | *5.0 | 10.1 | *6.2 | 22.1 | *3.0 | 12.9 | *6.7 | *6.7 | 15.6 | *8.0 | 17.0 | *4.69 |
| IL-2 | OOR< | *4.2 | *0.8 | 55.4 | *1.0 | *2.7 | OOR< | *0.6 | 10.8 | *0.6 | *5.5 | OOR< |
| IL-2R | 43.8 | 81.0 | 51.2 | 223.7 | 89.6 | 243.1 | 67.7 | *13.1 | 219.3 | *18.3 | 241.5 | 54.2 |
| IL-4 | *2.5 | *3.8 | *2.9 | *17.0 | *5.5 | *8.3 | *3.9 | *2.3 | *13.8 | *1.3 | *10.5 | OOR< |
| IL-5 | OOR< | *1.3 | *0.5 | 14.7 | *2.6 | 9.1 | *1.0 | OOR< | 7.9 | OOR< | 7.7 | OOR< |
| IL-6 | 56.5 | 78.4 | 40.9 | 1062.5 | 106.5 | 318.4 | 47.0 | 33.2 | 688.5 | 31.4 | 857.3 | 23.2 |
| IL-7 | OOR< | 6.3 | OOR< | 42.7 | 20.0 | 19.9 | OOR< | 6.3 | 23.4 | OOR< | 22.0 | 28.0 |
| IL-8 | 226.2 | 231.0 | 253.4 | 4904.6 | 827.4 | 1591.0 | 677.8 | 283.2 | 1023.9 | 84.4 | 794.9 | 66.0 |
| IP-10 | 161.4 | 766.7 | 307.3 | 6213.7 | 916.7 | 59779.1 | 510.1 | 156.9 | 393430.8 | 345.3 | 305579.5 | 79.2 |
| MCP-1 | 1660.6 | 1752.3 | 1280.8 | 18439.9 | 4437.4 | 1939.1 | 791.9 | 1598.9 | 10868.4 | 420.0 | 3157.4 | 888.7 |
| MIG | 82.9 | 302.1 | 179.1 | 4500.5 | 1360.6 | 3621.2 | 1342.1 | 380.7 | 3423.0 | 288.2 | 3823.6 | 29.3 |
| MIP-1α | 22.0 | 28.0 | 20.7 | 68.1 | 31.9 | 50.8 | 19.7 | *14.8 | 68.6 | *14.6 | 64.4 | *8.8 |
| MIP-1β | 26.3 | 33.8 | 26.1 | 213.8 | 49.7 | 106.1 | 24.2 | 16.8 | 126.8 | 22.3 | 52.6 | 13.6 |
| RANTES | *15.5 | OOR< | OOR< | 41.7 | 25.7 | OOR< | OOR< | OOR< | 68.5 | *1.0 | *12.5 | OOR< |
| TNF-α | OOR< | OOR< | OOR< | 19.9 | *1.6 | *6.3 | OOR< | OOR< | 11.0 | OOR< | *5.1 | OOR< |
| VEGF | 17.0 | 21.8 | 16.7 | 90.2 | 25.5 | 38.6 | 10.9 | 7.8 | 65.5 | OOR< | 70.0 | 14.1 |

OOR<, Out of Range (below)
*Value extrapolated beyond standard range

TABLE 10
UPN 109 CSF Cytokine Fold Change Analysis, ICV Cycles 7 through 11.

| Cytokine | C7D0 | C7D2 | C8D0 | C8D1 | C9D0 | C9D2 | C9D8 | C10D0 | C10D1 | C11D0 | C11D1 | C11D44 |
|---|---|---|---|---|---|---|---|---|---|---|---|---|
| EGF | 1.0 | 1.0 | 1.0 | 1.0 | 1.0 | 1.0 | 1.0 | 1.0 | 1.0 | 1.0 | 1.0 | 1.0 |
| Eotaxin | 1.0 | 1.1 | 1.1 | 3.0 | 1.4 | 1.8 | 1.1 | 1.1 | 3.0 | 1.1 | 2.9 | 1.2 |
| FGF | 1.0 | 1.1 | 1.2 | 2.0 | 0.6 | 0.4 | 0.7 | 0.9 | 1.7 | 1.0 | 1.9 | 1.6 |
| G-CSF | 1.0 | 2.7 | 1.7 | 9.3 | 4.1 | 5.5 | 2.6 | 0.9 | 9.8 | 0.5 | 9.9 | 1.6 |
| GM-CSF | 1.0 | 1.2 | 1.3 | 4.4 | 1.3 | 2.1 | 0.7 | 0.7 | 1.6 | 0.7 | 1.4 | 0.7 |
| HGF | 1.0 | 1.5 | 1.7 | 3.4 | 2.2 | 3.4 | 2.0 | 1.5 | 2.9 | 1.7 | 3.3 | 1.1 |
| IFN-α | 1.0 | 1.2 | 0.9 | 2.4 | 1.3 | 1.5 | 0.8 | 0.4 | 2.0 | 0.5 | 1.6 | 0.5 |
| IFN-γ* | 1.0 | 0.9 | 0.5 | 17.2 | 2.0 | 3.9 | 0.6 | 0.2 | 8.5 | 0.5 | 5.2 | 0.1 |
| IL-10* | 1.0 | 1.4 | 0.5 | 17.0 | 4.7 | 16.0 | 3.6 | 0.8 | 33.4 | 1.6 | 38.1 | 0.5 |
| IL-12 | 1.0 | 1.4 | 1.5 | 5.5 | 2.5 | 5.0 | 3.7 | 2.1 | 3.4 | 2.6 | 5.1 | 0.8 |
| IL-13 | 1.0 | 1.0 | 0.8 | 1.9 | 1.0 | 1.1 | 0.3 | 0.3 | 1.4 | 0.3 | 1.2 | 0.3 |
| IL-15 | 1.0 | 1.0 | 1.0 | 1.0 | 1.0 | 1.0 | 1.0 | 1.0 | 1.0 | 1.0 | 1.0 | 1.0 |
| IL-17 | 1.0 | 1.2 | 0.4 | 3.8 | 2.3 | 2.1 | 1.0 | 0.5 | 3.5 | 0.4 | 3.4 | 0.4 |
| IL-1Rα* | 1.0 | 0.7 | 1.1 | 8.1 | 4.8 | 14.0 | 7.1 | 12.1 | 22.2 | 1.1 | 22.8 | 5.2 |
| IL-1β | 1.0 | 2.0 | 1.2 | 4.4 | 0.6 | 2.6 | 1.3 | 1.3 | 3.1 | 1.6 | 3.4 | 0.9 |
| IL-2* | 1.0 | 7.0 | 1.3 | 92.3 | 1.7 | 4.5 | 1.0 | 1.0 | 18.0 | 1.0 | 9.2 | 1.0 |
| IL-2R | 1.0 | 1.8 | 1.2 | 5.1 | 2.0 | 5.6 | 1.5 | 0.3 | 5.0 | 0.4 | 5.5 | 1.2 |
| IL-4 | 1.0 | 1.5 | 1.2 | 6.8 | 2.2 | 3.3 | 1.6 | 0.9 | 5.5 | 0.5 | 4.2 | 0.5 |
| IL-5* | 1.0 | 2.6 | 1.0 | 29.4 | 5.2 | 18.2 | 2.0 | 1.0 | 15.8 | 1.0 | 15.4 | 1.0 |
| IL-6* | 1.0 | 1.4 | 0.7 | 18.8 | 1.9 | 5.6 | 0.8 | 0.6 | 12.2 | 0.6 | 15.2 | 0.4 |
| IL-7 | 1.0 | 1.0 | 1.0 | 6.8 | 3.2 | 3.2 | 1.0 | 1.0 | 3.7 | 1.0 | 3.5 | 4.4 |
| IL-8* | 1.0 | 1.0 | 1.1 | 21.7 | 3.7 | 7.0 | 3.0 | 1.3 | 4.5 | 0.4 | 3.5 | 0.3 |
| IP-10* | 1.0 | 4.8 | 1.9 | 38.5 | 5.7 | 370.4 | 3.2 | 1.0 | 2437.6 | 2.1 | 1893.3 | 0.5 |
| MCP-1* | 1.0 | 1.1 | 0.8 | 11.1 | 2.7 | 1.2 | 0.5 | 1.0 | 6.5 | 0.3 | 1.9 | 0.5 |
| MIG* | 1.0 | 3.6 | 2.2 | 54.3 | 16.4 | 43.7 | 16.2 | 4.6 | 41.3 | 3.5 | 46.1 | 0.4 |
| MIP-1α | 1.0 | 1.3 | 0.9 | 3.1 | 1.5 | 2.3 | 0.9 | 0.7 | 3.1 | 0.7 | 2.9 | 0.4 |
| MIP-1β | 1.0 | 1.3 | 1.0 | 8.1 | 1.9 | 4.0 | 0.9 | 0.6 | 4.8 | 0.8 | 2.0 | 0.5 |
| RANTES | 1.0 | 0.1 | 0.1 | 2.7 | 1.7 | 0.1 | 0.1 | 0.1 | 4.4 | 0.1 | 0.8 | 0.1 |
| TNF-α* | 1.0 | 1.0 | 1.0 | 12.4 | 1.0 | 3.9 | 1.0 | 1.0 | 6.9 | 1.0 | 3.2 | 1.0 |
| VEGF | 1.0 | 1.3 | 1.0 | 5.3 | 1.5 | 2.3 | 0.6 | 0.5 | 3.9 | 0.5 | 4.1 | 0.8 |

Bold values, ‘OOR<’ value from Table 10 was replaced with the lowest measurable value for that cytokine to allow for fold change calculation.
*Cytokines in which a >10 fold increase was observed at least once

These immunological changes in the CSF were local, as no significant changes in cytokine levels (Table 11), and no detectable CAR+ T cells by qPCR and flow cytometry (data not shown) in the peripheral blood were observed. The changes in the CSF could not be compared to changes in the resected cavity of tumor lesion 1 (T1) due to the inability to obtain cyst fluid from the cavity during the ICT treatment course.

TABLE 11
UPN 109 Serum Cytokine Analysis (pg/mL), ICV Cycles 7 through 11.

| Cytokine | C7D0 | C7D2 | C7D4 | C8D0 | C8D1 | C8D4 | C9D0 | C9D2 | C10D0 | C10D1 | C10D3 | C11D0 | C11D1 | C11D2 |
|---|---|---|---|---|---|---|---|---|---|---|---|---|---|---|
| EGF | 148.1 | 166.7 | 171.9 | 168.8 | 132.2 | 118.3 | 105.9 | 73.8 | 154.8 | 158.9 | 121.9 | 114.8 | 152.2 | 151.8 |
| Eotaxin | 110.1 | 116.8 | 112.6 | 101.2 | 83.7 | 133.8 | 152.1 | 156.4 | 172.1 | 167.9 | 143.2 | 147.3 | 197.7 | 168.3 |
| FGF | *5.3 | 8.3 | 6.8 | OOR< | OOR< | OOR< | OOR< | 14.7 | 17.4 | 22.7 | 20.2 | 14.8 | 15.3 | 15.9 |
| G-CSF | 211.6 | 236.7 | 284.5 | 229.3 | 208.2 | 208.2 | 210.8 | 230.1 | 216.7 | 334.3 | 221.8 | 282.9 | 207.4 | 241.6 |
| GM-CSF | *2.0 | *2.1 | *2.4 | *1.9 | *1.6 | *2.0 | *1.9 | *1.7 | *2.0 | *3.1 | *2.3 | *1.9 | *1.8 | *1.7 |
| HGF | 471.5 | 596.1 | 611.6 | 420.1 | 403.0 | 508.1 | 362.4 | 400.0 | 385.9 | 502.5 | 456.6 | 395.3 | 476.1 | 451.6 |
| IFN-α | 43.9 | 47.4 | 49.9 | 43.8 | 42.2 | 47.1 | 43.9 | 40.0 | 41.1 | 64.5 | 43.1 | 50.4 | 43.3 | 43.4 |
| IFN-γ | 53.0 | 52.5 | 56.4 | 52.2 | 52.1 | 55.5 | 52.2 | 44.8 | 45.6 | 58.5 | 47.0 | 54.8 | 49.7 | 50.7 |
| IL-10 | *2.9 | *3.9 | *3.4 | *0.8 | OOR< | *2.4 | *1.0 | *3.3 | *2.6 | *9.4 | *3.5 | *1.8 | *1.9 | *0.6 |
| IL-12 | 211.7 | 192.3 | 195.0 | 187.2 | 182.3 | 190.9 | 192.8 | 223.3 | 227.9 | 241.6 | 254.4 | 220.2 | 240.5 | 219.1 |
| IL-13 | 21.1 | 27.4 | 30.7 | 23.1 | 36.8 | 31.0 | 28.0 | 24.2 | 22.3 | 38.3 | 31.4 | 32.2 | 25.8 | 34.8 |
| IL-15 | OOR< | OOR< | OOR< | OOR< | OOR< | OOR< | OOR< | OOR< | OOR< | OOR< | OOR< | OOR< | OOR< | OOR< |
| IL-17 | *3.5 | *4.1 | *5.2 | OOR< | *1.8 | *0.7 | *0.8 | *3.3 | *4.6 | *11.5 | *5.2 | *7.3 | *4.4 | *4.4 |
| IL-1Rα | 112.7 | 145.3 | *68.2 | *94.3 | *73.8 | *64.5 | *58.2 | *64.9 | 101.1 | 133.8 | 96.3 | *68.7 | 107.9 | 105.8 |
| IL-1β | *2.1 | *4.6 | *4.9 | *1.0 | OOR< | OOR< | OOR< | 11.3 | 15.9 | 30.6 | 18.2 | 12.7 | 14.3 | 14.7 |
| IL-2 | *0.1 | *0.9 | *1.3 | *0.3 | OOR< | *0.2 | *0.3 | *0.4 | *0.9 | *5.2 | *1.2 | *1.8 | *0.9 | *1.0 |
| IL-2R | 372.0 | 391.2 | 438.5 | 352.6 | 273.9 | 272.7 | 241.9 | 304.9 | 312.2 | 363.8 | 314.8 | 338.0 | 296.4 | 314.5 |
| IL-4 | *8.9 | *11.5 | *13.5 | *9.0 | *10.4 | *10.5 | *10.1 | *10.2 | *8.7 | *20.9 | *11.1 | *13.8 | *9.7 | *10.8 |
| IL-5 | *1.6 | *2.0 | *3.2 | *0.2 | *1.5 | *1.1 | *0.8 | OOR< | OOR< | 5.1 | *0.2 | *2.2 | OOR< | OOR< |
| IL-6 | OOR< | *1.6 | *0.4 | OOR< | *0.7 | OOR< | OOR< | *2.5 | *2.6 | 7.1 | *4.2 | *2.9 | *3.9 | *3.0 |
| IL-7 | OOR< | OOR< | OOR< | OOR< | OOR< | OOR< | OOR< | OOR< | OOR< | OOR< | OOR< | OOR< | OOR< | OOR< |
| IL-8 | 49.4 | 130.2 | 88.4 | 96.4 | 56.3 | 17.1 | 43.6 | *9.4 | 32.8 | 93.1 | 112.1 | 18.3 | 30.1 | 59.0 |
| IP-10 | 33.9 | 23.5 | 17.2 | 11.7 | 9.0 | 11.5 | 15.0 | 12.7 | 17.4 | 33.6 | 16.3 | 15.3 | 23.2 | 18.9 |
| MCP-1 | 459.6 | 610.9 | 475.9 | 426.2 | 414.8 | 561.0 | 944.8 | 538.5 | 848.2 | 1074.3 | 703.0 | 954.9 | 950.0 | 826.3 |
| MIG | 141.3 | 108.1 | 50.6 | 8.0 | OOR< | 10.0 | 28.5 | 34.0 | 41.2 | 79.1 | 42.6 | 47.3 | 42.2 | 44.2 |
| MIP-1α | 58.4 | 58.4 | 62.2 | 51.1 | 49.2 | 53.4 | 53.1 | 47.1 | 55.8 | 81.2 | 57.7 | 64.6 | 52.7 | 54.1 |
| MIP-1β | 103.3 | 93.4 | 92.0 | 78.1 | 64.4 | 76.8 | 83.1 | 57.3 | 84.5 | 157.1 | 86.3 | 90.0 | 87.6 | 90.0 |
| RANTES | 11127.1 | 11965.0 | 14328.5 | 10584.5 | 12610.1 | 12415.5 | 12937.9 | 9221.7 | 8567.4 | 10428.1 | 8886.3 | 11117.8 | 9782.5 | 9771.1 |
| TNF-α | *1.2 | *2.1 | *4.5 | *2.3 | *2.6 | *2.1 | *2.3 | OOR< | OOR< | 7.2 | OOR< | *2.1 | OOR< | OOR< |
| VEGF | OOR< | OOR< | OOR< | OOR< | OOR< | OOR< | OOR< | OOR< | OOR< | OOR< | OOR< | OOR< | OOR< | OOR< |

OOR<, Out of Range (below)
*Value extrapolated beyond standard range

### Example 24: Patient Sample Processing and Analysis

Tumor resection material was collected through the COH department of Pathology according to the clinical protocol.

IL13Rα2 immunohistochemistry (IHC) was performed on 5 μm-sections of formalin-fixed paraffin-embedded specimens as previously described, and Ki67 JHC was similarly performed with the exception of antigen retrieval by heating @pH 8.0, and incubation with a 1:75 dilution of anti-K167 (Dako Corp). IL-13Rα2 immunoreactivity was scored by a clinical neuropathologist and quantified based on the percentage of tumor cells exhibiting weak (1+), moderate (2+), or strong (3+) intensity of cytoplasmic and golgi-like staining. The H score is obtained by the formula: (3×percentage of strongly staining cells)+(2×percentage of moderately staining cells)+percentage of weakly staining cells, giving a range of 0 to 300. The H score can be translated into the intensity scoring system described in the enrollment criteria as follows: 0 representing negative (H score 0), 1+ low (H score 1-100), 2+ moderate (H score 101-200) and 3+ high (H score 201-300). The criteria for inclusion was at least 20% of the cells scoring 1+ staining intensity (>20%, 1+), representing an H score of 20. Appropriate positive (testicular) and negative (prostate) controls were employed for IL-13Rα2 IHC staining. A “+” sign reflects the presence of membranous staining. This test has been performed at the Department of Pathology, City of Hope National Medical Center and is regarded as investigational for research. This Laboratory is certified under the Clinical Laboratory Improvement Amendments of 1988 (CLIA) as qualified to perform high complexity clinical laboratory testing.

Peripheral blood samples were collected in vacutainer tubes ±EDTA. Samples with EDTA were ficolled immediately upon receipt and peripheral blood mononuclear cells (PBMC) were frozen in Crystor CS5 at −80° C. and then transferred to liquid nitrogen for long term storage. Samples without EDTA were allowed to coagulate for 2-3 hours at room temperature; serum was collected by centrifugation, aliquoted in single use 100-200 μl aliquots and stored at −80° C. Cerebral spinal fluid (CSF) was collected from the ICV reservoir in a 3 cc syringe, spun down, and supernatants were aliquoted and stored at −80° C. The CSF cells were resuspended in HBSS−/− (Corning CellGro) with 2% FCS and sodium azide for immediate flow cytometric analysis, with the remaining cells resuspended and frozen in Cryostor CS4 at −80° C. and then transferred to liquid nitrogen for long term storage

Cell surface phenotyping of immune cells was performed by flow cytometry using fluorochrome conjugated antibodies specific for CD3, CD4, CD11b, CD14, CD19, CD27, CD28, CD62L, CD45RA, CD45RO, IL-13, TCR-α/β (BD Biosciences), KLRG1, CD15 (BioLegend), HLA-DR, PD1 (eBiosciences), CD8 (Fisher Scientific), LAG-3 (Lifespan Biosciences), CCR7, or TIM-3 (R&D Systems), and their respective isotype controls.

Research participant serum and CSF samples were analyzed by cytokine bead array. Assays were performed using the Human Cytokine 30-Plex Panel kit (Invitrogen) and a FLEXMAP 3D® (Luminex).

What is claimed is:

1. A method of treating a patient diagnosed with a malignancy of the central nervous system comprising introducing into the cerebrospinal fluid (CSF) of the patient a composition comprising an effective amount of T cells

2. The method of claim 1 wherein the T cells are autologous or allogenic T cells.

3. The method of claim 1 wherein the T cells have been manipulated ex vivo by one or more of: expansion, fractionation or transfection with a recombinant nucleic acid molecule.

4. The method of claim 3 wherein the T cells comprise cells that have been transfected with a recombinant nucleic acid molecule encoding a polypeptide that binds to a tumor cell antigen.

5. The method of claim 4 wherein the polypeptide is a chimeric antigen receptor.

6. The method of claim 1 wherein the composition is administered intraventricularly

7. The method of claim 1 wherein the composition is administered to the central canal of the spinal cord.

8. The method of claim 6 wherein the administration is to the left ventrical or the right ventrical.

9. The method of claim 1 wherein the composition comprises at least 1×10⁶ cells.

10. The method of claim 1 wherein a composition comprising T cells is administered at least two times.

11. The method of claim 10 wherein the wherein the administrations differ in the total number of T cells administered.

12. The method of claim 10 wherein the administrations escalate in dose.

13. The method of claim 10 wherein the administration de-escalate in dose.

14. The method of claim 1 wherein the T cells comprise CAR T cells expressing a chimeric antigen receptor.

15. The method of claim 1 wherein the T cells comprise autologous tumor infiltrating lymphocytes.

16. The method of claim 1 wherein the T cells comprise TCR-engineered T cells.

17. The method of claim 1 wherein the malignancy is a diffuse, infiltrating tumor.

18. The method of claim 1 wherein the malignancy is a primary brain tumor.

19. The method of claim 1 wherein one or more tumor foci decrease in size by at least 25%.

20. The method of claim 1 wherein the malignancy arose from a primary cancer selected from: breast cancer, lung cancer, head and neck cancer, and melanoma.

21. The method of claim 1 wherein the method is performed after tumor resection.

22. The method of claim 1 further comprising intratumoral administration of a composition comprising T cells.

23. The method of claim 1 wherein the malignancy is secondary brain tumor.

24. The method of claim 1 further comprising intratumoral administration of a composition comprising therapeutic T cells expressing a chimeric antigen receptor that binds a protein expressed on the surface of glioblastoma cells.

25. The method of claim 24 wherein the patient has previously undergone resection of a tumor lesion.

26. The method of claim 4 wherein the tumor antigen is selected from the group consisting of: IL13Rα2, HER2, PSCA, EGFR, EGFRvIII, EphA2, NY-ESO-1, and CD19.

27. The method of claim 1 wherein the T cells comprise both CD4+ cells and CD8+ cells.

28. The method of claim 1 wherein the T cells have undergone ex vivo expansion.

29. The method of claim 1 wherein the T cells comprise at least 10% Tcm cells.

30. The method of claim 14 wherein the CAR T cells target IL13Rα2 and comprise a nucleic acid molecule encoding a chimeric antigen receptor comprising: human IL-13 or a variant thereof having 1-10 amino acid modifications; a transmembrane domain selected from: a CD4 transmembrane domain or variant thereof having 1-10 amino acid modifications, a CD8 transmembrane domain or variant thereof having 1-10 amino acid modifications, a CD28 transmembrane domain or a variant thereof having 1-10 amino acid modifications, and a CD3ζ transmembrane domain or a variant thereof having 1-10 amino acid modifications; at least one costimulatory domain; and CD3ζ signaling domain of a variant thereof having 1-10 amino acid modifications.

31. The method of claim 30 wherein the costimulatory domain is selected from the group consisting of: a CD28 costimulatory domain or a variant thereof having 1-10 amino acid modifications, a 4IBB costimulatory domain or a variant thereof having 1-10 amino acid modifications and an OX40 costimulatory domain or a variant thereof having 1-10 amino acid modifications.

32. The method of claim 30 wherein the variant of a human IL-13 has 1-10 amino acid modification that increase binding specificity for IL13Rα2 versus IL13Rα1.

33. The method of claim 30 wherein the human IL-13 or variant thereof is an IL-13 variant comprising the amino acid sequence of SEQ ID NO:3 with 1 to 5 amino acid modifications, provided that the amino acid at position 11 of SEQ ID NO:3 is other than E.

34. The method of claim 31 wherein the chimeric antigen receptor comprises two different costimulatory domains selected from the group consisting of: a CD28 costimulatory domain or a variant thereof having 1-10 amino acid modifications, a 4IBB costimulatory domain or a variant thereof having 1-10 amino acid modifications and an OX40 costimulatory domain or a variant thereof having 1-10 amino acid modifications.

35. The method of claim 31 wherein the chimeric antigen receptor comprises two different costimulatory domains selected from the group consisting of: a CD28 costimulatory domain or a variant thereof having 1-2 amino acid modifications, a 4IBB costimulatory domain or a variant thereof having 1-2 amino acid modifications and an OX40 costimulatory domain or a variant thereof having 1-2 amino acid modifications.

36. The method of claim 30 wherein the chimeric antigen receptor comprises: human IL-13 or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ζ transmembrane domain or a variant thereof having 1-2 amino acid modifications; a costimulatory domain; and CD3ζ signaling domain of a variant thereof having 1-2 amino acid modifications.

37. The method of claim 30 wherein the chimeric antigen receptor comprises a spacer region located between the IL-13 or variant thereof and the transmembrane domain.

38. The method of claim 37 wherein the spacer region comprises an amino acid sequence selected from the group consisting of SEQ ID NO: 4, 14-20, 50 and 521.

39. The method of claim 30 wherein the chimeric antigen receptor comprises an amino acid sequence selected from the group consisting of SEQ ID NOs: 10 and 31-48.

40. The method of claim 30 wherein the chimeric antigen receptor binds HER2 and the central memory T cells comprise a nucleic acid molecule encoding a chimeric antigen receptor comprising: a HER2 targeting domain; a transmembrane domain selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications, and a CD3s transmembrane domain or a variant thereof having 1-5 amino acid modifications; a costimulatory domain selected from a CD28 costimulatory domain or a variant thereof having 1-5 amino acid modifications and a 4-IBB costimulatory domain or a variant thereof having 1-5 amino acid modifications; and CD3s signaling domain of a variant thereof having 1-5 amino acid modifications.

41. The method of claim 40 wherein the HER2 targeting domain is a HER2 scFv.

42. The method of claim 41 wherein the HER2 scFv comprising the amino acid sequence: DIQMTQSPSSLSASVGDRVTITCRASQDVNTAVAWYQQKPGKAPKLLIYSASFLYSGVPSRFS GSRSGTDFTLTISSLQPEDFATYYCQQHYTTPPTFGQGTKVEIKGSTSGGGSGGGSGGGGSSE VQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS or a variant thereof having 1 to 5 amino acid modifications.

43. The method of claim 40 wherein the chimeric antigen receptor comprises: a HER2 targeting sequence; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3s transmembrane domain or a variant thereof having 1-2 amino acid modifications; a costimulatory domain selected from a CD28 costimulatory domain or a variant thereof having 1-2 amino acid modifications and a 4-IBB costimulatory domain or a variant thereof having 1-2 amino acid modifications; and CD3s signaling domain of a variant thereof having 1-2 amino acid modifications.

44. The method of claim 40 wherein the nucleic acid molecule expresses a polypeptide comprising an amino acid sequence selected from SEQ ID NO: 26 and 27 or a variant thereof having 1-5 amino acid modifications.

45. A method of treating a patient diagnosed with a malignancy of the central nervous system comprising infusing a composition comprising an effective amount of T cells into an anatomical compartment of a patient diagnosed with a malignancy of the central nervous system, the anatomical compartment containing cerebrospinal fluid (CSF).

46. The method of claim 45 in which the anatomical compartment comprises a portion of a ventricular system.

47. The method of claim 45 in which the anatomical compartment comprises a portion of a central canal of a spinal cord.

48. The method of claim 45 in which the malignancy of the central nervous system includes a brain tumor.

49. The method of claim 45 in which the malignancy of the central nervous system includes a metastasized tumor.

50. The method of claim 45 in which the anatomical compartment contains a contiguous volume of at least about 50 mL of cerebrospinal fluid.

51. The method of claim 45 in which the anatomical compartment contains a contiguous volume of at least about 100 mL of cerebrospinal fluid.

52. The method of claim 45 in which the anatomical compartment contains a contiguous volume of at least about 150 mL of cerebrospinal fluid.

53. The method of claim 1 or claim 45 wherein the malignancy arises from a cancer selected from the group consisting of: primary CNS malignancies and secondary malignancies arising from a cancer located elsewhere, for example. Acute Lymphoblastic Leukemia (ALL), Acute Myeloid Leukemia (AML), Adrenocortical, Carcinoma, AIDS-Related Cancers, Anal Cancer, Appendix Cancer, Astrocytomas, Atypical Teratoid/Rhabdoid Tumor, Central Nervous System, Basal Cell Carcinoma, Bile Duct Cancer, Bladder Cancer, Bone Cancer, Osteosarcoma and Malignant Fibrous Histiocytoma, Brain Stem Glioma, Brain Tumors, Breast Cancer, Bronchial Tumors, Burkitt Lymphoma, Carcinoid Tumors, Central Nervous System Cancers, Cervical Cancer, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Embryonal Tumors, Central Nervous System, Endometrial Cancer, Ependymoblastoma, Ependymoma, Esophageal Cancer, Esthesioneuroblastoma, Ewing Sarcoma Family of Tumors Extracranial Germ Cell Tumor, Extragonadal Germ Cell Tumor Extrahepatic Bile Duct Cancer, Eye Cancer Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST)—see Soft Tissue Sarcoma, Germ Cell Tumor, Gestational Trophoblastic Tumor, Glioma, Hairy Cell Leukemia, Head and Neck Cancer, Heart Cancer, Hepatocellular (Liver) Cancer, Histiocytosis, Hodgkin Lymphoma, Hypopharyngeal Cancer, Intraocular Melanoma, Islet Cell Tumors (Endocrine Pancreas), Kaposi Sarcoma, Kidney cancer, Langerhans Cell Histiocytosis, Laryngeal Cancer, Leukemia, Lip and Oral Cavity Cancer, Liver Cancer (Primary), Lobular Carcinoma In Situ (LCIS), Lung Cancer, Lymphoma, Macroglobulinemia, Male Breast Cancer, Malignant Fibrous Histiocytoma of Bone and Osteosarcoma, Medulloblastoma, Medulloepithelioma, Melanoma, Merkel Cell Carcinoma, Mesothelioma, Metastatic Squamous Neck Cancer with Occult Primary Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma, Multiple, Myeloproliferative Disorders, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma and Malignant Fibrous Histiocytoma of Bone, Ovarian Cancer, Pancreatic Cancer, Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Sezary Syndrome, Small Cell Lung Cancer, Small Intestine Cancer, Soft Tissue Sarcoma, Squamous Cell Carcinoma, Squamous Neck Cancer, Stomach (Gastric) Cancer, Supratentorial Primitive Neuroectodermal Tumors, T-Cell Lymphoma, Cutaneous, Testicular Cancer, Throat Cancer, Thymoma and Thymic Carcinoma, Thyroid Cancer, Transitional Cell Cancer of the Renal Pelvis and Ureter, Trophoblastic Tumor, Ureter and Renal Pelvis Cancer, Urethral Cancer, Uterine Cancer, Uterine Sarcoma, Vaginal Cancer, Vulvar Cancer, Waldenstrom Macroglobulinemia, and Wilms Tumor.

54. The method of claim 1 or 45, wherein the malignancy comprises a tumor.

55. The method of claim 54, wherein treatment results in at least a 50% reduction in tumor volume.

56. The method of claim 54, wherein treatment results in at least a 60% reduction in tumor volume.

57. The method of claim 54, wherein treatment results in at least a 70% reduction in tumor volume.

58. The method of claim 54, wherein treatment results in at least an 80% reduction in tumor volume.

59. The method of claim 54, wherein treatment results in at least an 90% reduction in tumor volume.

60. The method of claim 1 or 45, wherein treatment results in elimination of the malignancy.

61. The method of claim 1 or 45, wherein the patient does not experience any grade 3 or higher toxicity.

62. The method of claim 1 or 45, wherein the patient was administered a regimen of steroids prior to treatment with the composition comprising an effective amount of T cells.

63. The method of claim 60, wherein the regimen of steroids is reduced to a lower dose following the treatment.

64. The method of claim 1 or 45, wherein the patient has a 1-year survival rate of at least 50%.

65. The method of claim 1 or 45, wherein the patient has a 2-year survival rate of at least 50%.

66. The method of claim 1 or 45, wherein the patient has a 5-year survival rate of at least 50%.

67. The method of claim 1 or 45, wherein the patient has an increased life expectancy compared to a patient receiving standard of care (SOC) treatment, including radiation therapy, small molecule drug therapy, antibody therapeutics, or combinations thereof.

68. The method of claim 67, in which the patient receiving SOC treatment can expect to survive about 15 months from initial diagnosis (overall survival or OS) whereas the patient receiving the claimed treatment can expect an OS of 25, 26, 27, 28, 29, 30, 31, 32, 33, 34, 35, 36 months or more.

69. The method of claim 68, in which the patient receiving the claimed treatment can expect an OS of 42, 48, 54, 60, 66, 72, 78, 84, 90 months or more.

70. The method of claim 1 or 45, wherein the composition comprises at least 2×10⁶ T cells.

71. The method of claim 1 or 45, wherein the composition comprises at least 10×10⁶ T cells.

72. The method of claim 1 or 45, wherein the method comprises repeating administration of the composition at least five times.

73. The method of claim 1 or 45, wherein the method comprises repeating administration of the composition at least ten times.

74. The method of claim 1 or 45, wherein the method comprises repeated administrations of the composition.

75. The method of claim 74, wherein the patient receives a total dose of at least 90×10⁶ T cells.

76. The method of claim 74, wherein administration is repeated once a week.

77. The method of claim 74, wherein administration is repeated once every two weeks.

78. The method of claim 74, wherein the repeated administrations are received over a fifteen week period.

79. A method of increasing a level of at least one cytokine or chemokine in the cerebrospinal fluid (CSF) of a patient comprising, administering a composition comprising an effective amount of T cells into the CSF of a patient with a malignancy of the central nervous system, wherein the level of at least one cytokine or chemokine in the CSF is increased following administration of the composition comprising an effective amount of T cells compared to a baseline level of the at least one cytokine or chemokine prior to the administration.

80. The method of claim 79, wherein the level of the at least one cytokine or chemokine in the CSF following the administration is increased 10-fold compared to the baseline level.

81. The method of claim 79, wherein the level of at least one cytokine or chemokine in the CSF following the administration is increased 5-fold compared to the baseline level.

82. The method of claim 79, wherein the level of at least five cytokines or chemokines is increased following administration of the composition comprising an effective amount of T cells compared to a baseline level of the at least five cytokines or chemokines prior to the administration.

83. The method of claim 79, wherein the level of at least ten cytokines or chemokines is increased following administration of the composition comprising an effective amount of T cells compared to a baseline level of the at least ten cytokines or chemokines prior to the administration.

84. The method of claim 79, wherein the at least one cytokine or chemokine comprises EGF, Eotaxin, FGF, G-CSF, GM-CSF, HGF, IFN-α, IFN-γ, IL-10, IL-12, IL-13, IL-15, IL-17, IL-1Rα, IL-1β, IL-2, IL-2R, IL-4, IL-5, IL-6, IL-7, IL-8, IP-10, MCP-1, MIG, MIP-1α, MIP-1β, RANTES, TNF-α, or VEGF.

85. The method of claim 79, wherein the at least one cytokine or chemokine comprises IFNγ, TNF, IL-2, IL-10, IL-5, IL-6, IL-8, CXCL9/MIG, CXCL10/IP-10, CCR2/MCP-1, or IL-1Rα.

86. The method of claim 79, wherein the at least one cytokine or chemokine comprises IFNγ, TNF, IL-2, IL-10, IL-5, IL-6, IL-8, CXCL9/MIG, CXCL10/IP-10, CCR2/MCP-1, IL-1Rα, G-CSF, IL-12, IL2-R, IL-4, IL-7, or MIP-1b.

87. The method of claim 79, wherein the increased level of at least one cytokine includes a local increase in the level of the at least one cytokine.

88. The method of claim 79, wherein the composition is administered intraventricularly.

89. The method of claim 79, wherein the T cells are autologous or allogenic T cells.

90. The method of claim 79, wherein the T cells have been manipulated ex vivo by one or more of: expansion, fractionation or transfection with a recombinant nucleic acid molecule.

91. The method of claim 90, wherein the T cells comprise cells that have been transfected with a recombinant nucleic acid molecule encoding a polypeptide that binds to a tumor cell antigen.

92. The method of claim 91, wherein the polypeptide is a chimeric antigen receptor.

93. The method of claim 92, wherein the chimeric antigen receptor is specific for IL-13R.

94. A method of sustaining for at least about five days an increased number of T cells, compared to a baseline number, observed in a cerebrospinal fluid (CSF) of a patient diagnosed with a malignancy of a central nervous system, comprising infusing an effective amount of T cells into a CSF of a patient diagnosed with a malignancy of a central nervous system, in which an increased number of T cells observed, compared to a baseline number observed prior to the infusion step, is sustained for at least about five days.

95. The method of claim 94, in which an effective amount of T cells ranges from about 1×10⁶ cells to about 100×10⁶ cells.

96. The method of claim 94, in which an effective amount of T cells ranges from about 2×10⁶ cells to about 50×10⁶ cells.

97. The method of claim 94, in which the increased number of T cells observed is sustained for at least about six days.

98. The method of claim 94, in which the number of T cells observed does not return to the baseline number for about seven days.

99. The method of claim 94, in which the T cells observed include infused T cells.

100. The method of claim 94, in which the T cells observed include endogenous T cells.

101. A method of increasing a number of T cells in the cerebrospinal fluid (CSF) of a patient comprising, administering a composition comprising an effective amount of T cells into the CSF of a patient with a malignancy of the central nervous system, wherein the number of T cells detectable in the CSF is increased compared to pre-administration levels.

102. The method of claim 101, wherein the number of T cells detectable in the CSF is increased compared to pre-administration levels for up to seven days following administration.

103. The method of claim 101, wherein the T cells detectable in the CSF comprise endogenous T cells and CAR-expressing T cells.

104. The method of claim 101, wherein the T cells detectable in the CSF comprise endogenous Type 1 T cells.

105. The method of claim 101, wherein the T cells detectable in the CSF comprise endogenous Type 2 T cells.

106. The method of claim 101, wherein the T cells detectable in the CSF comprise CD3+ T cells.

107. The method of claim 101, wherein the T cells detectable in the CSF comprise CD14+ CD11b+ HLA-DR+ mature myeloid populations.

108. The method of claim 101, wherein CD19+ B cells and CD11b+ CD15+ granulocytes are detectable in the CSF following administration of the composition.

109. The method of claim 101, wherein reactive lymphocytes, monocytes, and macrophages are detectable in the CSF following administration of the composition.

110. The method of claim 101, wherein a local increase in the level of the at least one cytokine is observed.

111. The method of claim 101, wherein the composition is infused intraventricularly.

112. The method of claim 101, wherein the T cells are autologous or allogenic T cells.

113. The method of claim 101, wherein the T cells have been manipulated ex vivo by one or more of: expansion, fractionation or transfection with a recombinant nucleic acid molecule.

114. The method of claim 113, wherein the T cells comprise cells that have been transfected with a recombinant nucleic acid molecule encoding a polypeptide that binds to a tumor cell antigen.

115. The method of claim 114, wherein the polypeptide is a chimeric antigen receptor.

116. The method of claim 115, wherein the chimeric antigen receptor is specific for IL-13R.

117. A method of determining a suitability of a patient diagnosed with a malignancy for treatment with an IL-13Rα2-specific CAR T cell comprising, determining if a score attributed to a sample from the patient exhibits IL-13Rα2 expression above a predetermined threshold.

118. The method of claim 117, the score attributed to the sample is calculated by determining the immunoreactivity of a resected tumor sample from a patient diagnosed with a malignancy by immunohistochemically staining the sample with a marker of IL-13Rα2, analyzing the strength of the staining, and calculating a score based on the strength of the staining, wherein a score that corresponds to moderate to strong staining intensity in the sample indicates that treatment with an IL-13Rα2-specific CAR T cell is suitable for the patient.

119. The method of claim 117, wherein the score comprises counting the number of cells that have a weak, moderate, or strong staining intensity and assigning each intensity a weight.

120. The method of claim 117, wherein an expression of Ki67 in the sample is also determined by immunohistochemical staining.

121. A method of treating a patient with a malignancy comprising, administering to a patient diagnosed with a malignancy a composition comprising an effective dose of IL-13Rα2-specific CAR T cells, wherein the patient expresses IL-13Rα2 above a predetermined threshold.

122. The method of claim 120, wherein the predetermined threshold of IL-13Rα2 expression was previously identified as being suitable for a treatment comprising IL-13Rα2-specific CAR T cell therapy.

123. The method of claim 54 wherein the patient is suffering from multifocal glioblastoma.

124. The method of claim 1 or claim 45 wherein the treatment increases the level of CXCL9 or CXCL10 or both in cerebral spinal fluid.


SEQUENCE LISTING (59 sequences)

SEQ ID NO: 1 - protein, 889 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 2 - protein, 22 aa, synthetic construct
MLLLVTSLLLCELPHPAFLLIP

SEQ ID NO: 3 - protein, 112 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 4 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 22 aa, Homo sapiens
MALIVLGGVAGLLLFIGLGIFF

SEQ ID NO: 6 - protein, 42 aa, synthetic construct
KRGRKKLLYIFKQPFMRPVQTTQEEDGCSCRFPEEEEGGCEL

SEQ ID NO: 7 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 24 aa, synthetic construct
LEGGGEGRGSLLTCGDVEENPGPR

SEQ ID NO: 9 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 10 - protein, 889 aa, synthetic construct; identical to SEQ ID NO: 1

SEQ ID NO: 11 - protein, 405 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCEL

SEQ ID NO: 12 - DNA, 9515 bp, synthetic construct
  source 1..9515: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctggtgaccagcctgctgctgtgcgagctgccccaccccgcctttctgctgatccctggccccgtgccccctagcaccgccctgcgctacctgatcgaggaactggtgaacatcacccagaaccagaaagcccccctgtgcaacggcagcatggtgtggagcatcaacctgaccgccggcatgtactgtgccgccctggaaagcctgatcaacgtgagcggctgcagcgccatcgagaaaacccagcggatgctgtccggcttctgcccccacaaggtgtccgccggacagttcagcagcctgcacgtgcgggacaccaagatcgaggtggcccagttcgtgaaggacctgctgctgcacctgaagaagctgttccgggagggccggttcaacgagagcaagtacggccctccctgccccccttgccctgccccagagttcgagggcggacccagcgtgttcctgttcccccccaagcccaaggacaccctgatgatcagccggacccctgaggtgacctgcgtggtggtggacgtgagccaggaagatcctgaggtccagttcaattggtacgtggacggcgtggaggtgcacaacgccaagaccaagcccagggaagagcagttccagagcacctaccgggtggtgtccgtgctgaccgtgctgcaccaggactggctgaacggcaaagaatacaagtgcaaggtgtccaacaagggcctgcccagcagcatcgagaaaaccatcagcaaggccaagggccagcctcgggagccccaggtgtacaccctgcccccttcccaggaagagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggaaggcaacgtctttagctgcagcgtgatgcacgaggccctgcacaaccactacacccagaagagcctgtccctgagcctgggcaagatggccctgatcgtgctgggcggcgtggccgggctgctgctgttcatcggcctgggcatctttttcaaacggggcagaaagaaactcctgtatatattcaaacaaccatttatgagaccagtacaaactactcaagaggaagatggctgtagctgccgatttccagaagaagaagaaggaggatgtgaactgggcggagggcgggtgaagttcagccggtccgccgacgcccctgcctaccagcagggccagaaccagctgtacaacgagctgaacctgggcaggcgggaggaatacgacgtgctggacaagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagataatctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 13 - DNA, 8732 bp, synthetic construct
  source 1..8732: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgagagcaagtacggccctccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaggtgcacaacgccaagaccaagcccagggaagagcagttcaacagcacctaccgggtggtgtccgtgctgaccgtgctgcaccaggactggctgaacggcaaagaatacaagtgcaaggtgtccaacaagggcctgcccagcagcatcgagaaaaccatcagcaaggccaagggccagcctcgggagccccaggtgtacaccctgcccccttcccaggaagagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggaaggcaacgtctttagctgcagcgtgatgcacgaggccctgcacaaccactacacccagaagagcctgtccctgagcctgggcaagatggccctgatcgtgctgggcggcgtggccgggctgctgctgttcatcggcctgggcatctttttccgggtgaagttcagccggtccgccgacgcccctgcctaccagcagggccagaaccagctgtacaacgagctgaacctgggcaggcgggaggaatacgacgtgctggacaagcggagaggccgggaccctgagatgggcggcaagcccaggcggaagaaccctcaggaaggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaggtgacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 14 - protein, 10 aa, synthetic construct
GGGSSGGGSG

SEQ ID NO: 15 - protein, 12 aa, synthetic construct
ESKYGPPCPPCP

SEQ ID NO: 16 - protein, 22 aa, synthetic construct
ESKYGPPCPPCPGGGSSGGGSG

SEQ ID NO: 17 - protein, 39 aa, synthetic construct
IEVMYPPPYLDNEKSNGTIIHVKGKHLCPSPLFPGPSKP

SEQ ID NO: 18 - protein, 48 aa, synthetic construct
AKPTTTPAPRPPTPAPTIASQPLSLRPEACRPAAGGAVHTRGLDFACD

SEQ ID NO: 19 - protein, 45 aa, synthetic construct
TTTPAPRPPTPAPTIASQPLSLRPEACRPAAGGAVHTRGLDFACD

SEQ ID NO: 20 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 21 aa, Homo sapiens
LCYLLDGILFIYGVILTALFL

SEQ ID NO: 22 - protein, 27 aa, Homo sapiens
FWVLVVVGGVLACYSLLVTVAFIIFWV

SEQ ID NO: 23 - protein, 21 aa, Homo sapiens
IYIWAPLAGTCGVLLLSLVIT

SEQ ID NO: 24 - protein, 23 aa, Homo sapiens
IYIWAPLAGTCGVLLLSLVITLY

SEQ ID NO: 25 - protein, 24 aa, Homo sapiens
IYIWAPLAGTCGVLLLSLVITLYC

SEQ ID NO: 26 - protein, 27 aa, Homo sapiens
IISFFLALTSTALLFLLFFLTLRFSVV

SEQ ID NO: 27 - protein, 41 aa, Homo sapiens
RSKRSRLLHSDYMNMTPRRPGPTRKHYQPYAPPRDFAAYRS

SEQ ID NO: 28 - protein, 41 aa, Homo sapiens
RSKRSRGGHSDYMNMTPRRPGPTRKHYQPYAPPRDFAAYRS

SEQ ID NO: 29 - protein, 42 aa, Homo sapiens; identical to SEQ ID NO: 6

SEQ ID NO: 30 - protein, 42 aa, synthetic construct
ALYLLRRDQRLPPDAHKPPGGGSFRTPIQEEQADAHSTLAKI

SEQ ID NO: 31 - protein, 362 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNAKPTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 410 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNAKPTTTPAPRPPTPAPTIASQPLSLRPEACRPAAGGAVHTRGLDFACDFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 442 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 541 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 373 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGSSGGGSGMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 385 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPGGGSSGGGSGMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 492 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 592 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 340 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNAKPTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 388 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNAKPTTTPAPRPPTPAPTIASQPLSLRPEACRPAAGGAVHTRGLDFACDFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 420 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 519 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 351 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGSSGGGSGMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 363 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPGGGSSGGGSGMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 470 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 570 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 363 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNAKPTTTPAPRPPTPAPTIASQPLSLRPEACRPAAGGAVHTRGLDFACDIYIWAPLAGTCGVLLLSLVITGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 341 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNAKPTTTPAPRPPTPAPTIASQPLSLRPEACRPAAGGAVHTRGLDFACDIYIWAPLAGTCGVLLLSLVITGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 245 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 50 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 12 aa, synthetic construct
ESKYGPPCPSCP

SEQ ID NO: 53 - protein, 1027 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 54 - protein, 658 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 1021 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 56 - protein, 652 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 28 aa, Homo sapiens
MFWVLVVVGGVLACYSLLVTVAFIIFWV

SEQ ID NO: 58 - protein, 1027 aa, synthetic construct; identical to SEQ ID NO: 53

SEQ ID NO: 59 - protein, 1021 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVFVHNAKTKPREEQFQSTYRVVSVLTVLHQDWLNGKFYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR